(19) United States
(12) Patent Application Publication
(10) Pub. No.: US 2010/0069244 A1
(43) Pub. Date: Mar. 18, 2010

(54) Pyridine Compounds for Combating Pests

(75) Inventors: Delphine Breuninger, Bobenheim-Roxheim (DE); Michael Puhl, Lampertheim (DE); Lilliana Parra Rapado, Offenburg (DE); Michael Rack, Eppelheim (DE); David G. Kuhn, Apex, NC (US); Deborah L. Culbertson, Fuquay Varina, NC (US); Douglas D. Anspaugh, Apex, NC (US)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/516,634

(22) PCT Filed: Nov. 28, 2007
(86) PCT No.: PCT/EP2007/062961

§ 371 Date: May 28, 2009

Related U.S. Application Data
(60) Provisional application No. 60/867,642, filed on Nov. 29, 2006.

(51) Int. Cl. A01N 25/26; A01N 43/44; C07D 401/02; A01N 43/40; A01P 15/00

(52) U.S. Cl. 504/100; 514/210.2; 546/268.1; 514/343; 546/276.4

(57) ABSTRACT

The present invention relates to a method of combating pest, which method comprises contacting said pest or their food supply, habitat, breeding ground or their locus with a 3-pyridyl compound of the formula (I). In formula (I) n is 1 or 2, y is 0 or 1 and X, R¹, R², R³, R⁴, R⁵ and R⁶ are as defined in the claims and in the specification. The invention also relates to novel 3-pyridyl compounds which are useful for combating animal pests, in particular insects and to pesticidal composition, which comprises at least one 3-pyridyl compound and an agriculturally acceptable carrier.

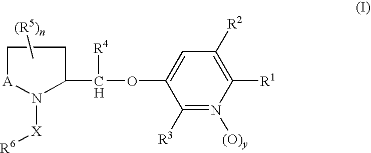

### BACKGROUND OF THE INVENTION

The present invention relates to a method of combating pest, which method comprises contacting said pest or their food supply, habitat, breeding ground or their locus with a 3-pyridyl compound. The invention also relates to novel 3-pyridyl compounds which are useful for combating animal pests, in particular insects and to pesticidal composition, which comprises at least one 3-pyridyl compound and an agriculturally acceptable carrier.

In spite of commercial pesticides available today, damage to crops, both growing and harvested, the damage of non-living material, in particular cellulose based materials such as wood or paper, caused by arthropod and nematode pests still occur. Therefore, there is continuing need to provide compounds which are useful for combating such pests.

WO 94/08992, WO 96/40682 and WO 98/25920, inter alia, mention pyridyloxymethylether compounds of the general formula A

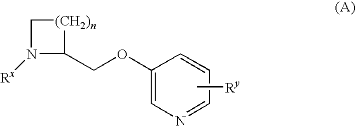

R^{x} is H or alkyl and wherein R^{y} is in most cases halogen or methyl. These compounds are mentioned to be useful for preparing pharmaceutical compositions for selectively controlling synaptic transmissions in mammals, or to be useful as analgetics, as well as neuronal cell death preventers and anti-inflammatories. WO 99/32480, among a large number of compounds, describes 3-pyridyl derivates in which the nitrogen atom of the azetidine or prolinol ring is substituted by hydrogen, allyl or C₁-C₆-alkyl. It also discloses in a general manner prodrug moieties which may be bonded to the nitrogen atom and which may be selected from numerous examples, inter alia forming an amide function together with the nitrogen atom.

WO 97/46554 and U.S. Pat. No. 5,629,325 describe similar 3-pyridyloxymethylether compounds, wherein the pyridine ring carries in the 5-position a radical L-R^{z}, wherein L is selected from a chemical bond, —C≡C—(C₀-C₆-alkylene)-, —(CH═CH)ₚ—(C₀-C₆-alkylene)- with p being 1 or 2, —CH═CH—C(O)—(C₀-C₆-alkylene)-, —C(O)—(C₀-C₆-alkylene)- and —CH₂-M-C(O)—(C₀-C₆-alkylene)- with M being CH₂ or NH and R^{z} is selected from hydrogen, optionally substituted C₁-C₈-alkyl, C₁-C₆-haloalkyl, C₁-C₆-hydroxyalkyl, C₁-C₆-alkoxy, amino, C₁-C₆-alkylamino, C₁-C₆-dialkylamino, an azacycle attached to L through a nitrogen atom, phenyl, naphthyl, biphenyl, furyl, thienyl, pyridinyl, pyridazinyl, pyrimidinyl, pyrrolyl, pyrazolyl, imidazolyl, indolyl, thiazolyl, oxazolyl, isoxazolyl, thiadiazolyl, oxadiazolyl, quinolinyl, and isoquinolyl, wherein alkyl and the 19 last mentioned radicals may carry one or two substituents, selected from C₁-C₆-alkyl, C₁-C₆-haloalkyl, C₁-C₆-alkoxy-C₁-C₆-alkyl, C₁-C₆-alkoxy, C₁-C₆-alkoxy-C₁-C₆-alkoxy, halogen, cyano, hydroxy, amino, C₁-C₆-alkylamino, carboxyl and C₁-C₆-alkoxycarbonyl. The compounds are taught to be useful for selectively controlling synaptic transmission.

WO 01/80844, WO 02/89800 and US 2003/0069272 teach the use of compounds A for the treatment of dry eye disease, vaginal dryness or for altering the amount or composition of synovials fluids secreted from joints. However, these documents do not mention how to obtain these compounds.

The unpublished patent application U.S. 60/762,305 of the applicant company describes pesticidal pyridin-3-yloxymethyl-pyrrolidine compounds and pyridin-3-yloxymethyl-azetidine compounds, wherein the nitrogen atom of the azetidine or prolinol ring, respectively, carry an acyl or thioacyl group.

The unpublished patent application PCT/EP2006/004992 of the applicant company describes pyridin-3-yloxymethyl-pyrrolidine compounds and pyridin-3-yloxymethyl-azetidine compounds, in which the nitrogen atom of the pyrrolidine ring or the azetidine ring, respectively, are substituted by a radical —O—R^{z}, with R^{z} being hydrogen, optionally substituted alkyl, optionally substituted alkenyl, optionally substituted alkynyl, optionally substituted cycloalkyl, optionally substituted phenyl or acyl. The compounds are used for combating pests, in particular arthropods and nematodes, and for protecting materials against infestation and/or destruction by said pests.

### SUMMARY OF THE INVENTION

It is an object of the present invention to provide compounds that have a good activity against harmful arthropod pests, such as insects and arachnids, or nematodes, and which thus are useful for combating said pests.

The inventors of the present application, surprisingly, found that this object is achieved by compounds of the formula I as defined below and by the salts thereof:

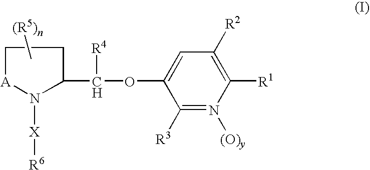

wherein
A is a chemical bond, CH₂ or a group CHR^{5a};
X is a chemical bond or C₁-C₃-alkylene, which may carry 1 or 2 identical or different radicals R⁷;
y is 0 or 1;
n is 0, 1 or 2;
R¹ and R² are each independently hydrogen, halogen, cyano, nitro, C₁-C₆-alkyl, which may be substituted by one OH, phenyl or CN radical, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, C₁-C₆-alkyl-C₃-C₈-cycloalkyl, ORⁱ, SRⁱ, S(═O)Rⁱ, S(═O)₂Rⁱ, NRⁱR^{j}, NR^{j}C(═O)Rⁱ, C(═O)Rⁱ, O—C(O)Rⁱ, C(═O)ORⁱ, SiRⁱ_{z}R^{j}_{3-z} with z being 0, 1, 2 or 3,
phenyl or a 3-, 4-, 5-, 6- or 7-membered heterocycle containing 1, 2, 3 or 4 heteroatoms selected from oxygen, nitrogen and sulfur, wherein phenyl and the heterocycle may be unsubstituted or may carry 1, 2, or 3 substituents selected from halogen, amino, cyano, Rⁱ, ORⁱ, SRⁱ, NRⁱR^{j}, NR^{j}C(═O)Rⁱ, C(═O)Rⁱ, O—C(O)Rⁱ, C(═O)ORⁱ, SO₂Rⁱ, and nitro;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, C₁-C₆-haloalkoxy, C₁-C₄-alkoxy-C₁-C₆-alkyl, C₁-C₄-alkylthio-C₁-C₆-alkyl, C₁-C₄-alkylsulfonyl-C₁-C₆-alkyl, C₁-C₄-haloalkoxy-C₁-C₆-alkyl, C₁-C₄-haloalkylthio-C₁-C₆-alkyl, (C₁-C₄-alkoxy)carbonyl-C₁-C₆-alkyl, (C₁-C₄-alkyl)amino-C₁-C₆-alkyl, di(C₁-C₄-alkyl)amino-C₁-C₆-alkyl, C₃-C₆-cycloalkyl-C₁-C₆-alkyl, phenyl-C₁-C₈-alkyl, or C₁-C₆-alkyl which is substituted with 1 to 3 cyano groups;
R² may also be a radical of the formula Z—R^{y}, wherein Z is selected from —C≡C—(C₀-C₆-alkylene)-, —(CH═CH)ₚ—(C₀-C₆-alkylene)- with p being 1 or 2, —CH═CH—C(O)—(C₀-C₆-alkylene)-, —C(O)—(C₀-C₆-alkylene)- and —CH₂—Z′—C(O)—(C₀-C₆-alkylene)- with Z′ being CH₂ or NH and R^{y} is selected from hydrogen, C₁-C₈-alkyl, C₁-C₆-haloalkyl, C₁-C₆-hydroxyalkyl, C₁-C₆-alkoxy, amino, C₁-C₆-alkylamino, C₁-C₆-dialkylamino, 5 to 7 membered, azaheterocyclyl, attached to Z via its nitrogen atom, phenyl, naphthyl, biphenyl, furyl, thienyl, pyridinyl, pyridazinyl, pyrimidinyl, pyrrolyl, pyrazolyl, imidazolyl, indolyl, thiazolyl, oxazolyl, isoxazolyl, thiadiazolyl, oxadiazolyl, quinolinyl, and isoquinolyl, wherein alkyl and the 19 last mentioned radicals may carry one or two substituents selected from C₁-C₆-alkyl, C₁-C₆-haloalkyl, C₁-C₆-alkoxy-C₁-C₆-alkyl, C₁-C₆-alkoxy, C₁-C₆-alkoxy-C₁-C₆-alkoxy, halogen, cyano, hydroxy, amino, C₁-C₆-alkylamino, carboxyl and C₁-C₆-alkoxycarbonyl;
R³ is hydrogen, halogen or C₁-C₆-alkyl;
R⁴ is hydrogen or C₁-C₆-alkyl;
R⁵ is selected from halogen, OH, cyano, C₁-C₆-alkyl and C₁-C₆-haloalkyl and for n being 2 R⁵ may be identical or different;
R^{5a} is selected from halogen, cyano, C₁-C₆-alkyl and C₁-C₆-haloalkyl, in particular halogen, cyano, methyl or halomethyl;
R⁶ is C₁-C₁₀-alkyl, C₂-C₁₀-alkenyl, C₂-C₁₀-alkynyl, wherein the last three mentioned radicals may be unsubstituted or may carry 1, 2 or 3 identical or different radicals R^{a},
C₁-C₁₀-haloalkyl, C₂-C₁₀-haloalkenyl, C₂-C₁₀-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from oxygen, nitrogen and sulfur, wherein C₃-C₈-cycloalkyl, C₃-C₈-cycloalkenyl, phenyl and the heterocycle may be unsubstituted or may carry 1, 2, 3, 4 or 5 substituents R^{b}, wherein C₃-C₈-cycloalkyl, C₃-C₈-cycloalkenyl, phenyl and the heterocycle may also be fused to 1 or 2 rings selected from phenyl and a 5- to 6-membered heterocycle which may contain 1, 2 or 3 heteroatoms selected from oxygen, nitrogen and sulfur,
R^{a} is selected from cyano, nitro, C₃-C₈-cycloalkyl, C₃-C₈-halocycloalkyl, C₃-C₈-cycloalkenyl, C₃-C₈-halocycloalkenyl, OR^{k}, SR^{k}, S(═O)R^{k}, S(═O)₂R^{k}, NR^{m}Rⁿ, N(OR^{k})R^{m}, S(═O)₂NR^{m}Rⁿ, O—C(═O)R^{k}, C(═O)R^{k}, C(═O)OR^{k}, C(═O)NR^{m}Rⁿ, C(═NOR^{k})R^{m}, NR^{k}C(=G)R^{m}, N(OR^{k})C(=G)R^{m}, N[C(=G)R^{k}][C(=G)R^{m}], NR^{k}C(=G)OR^{m}, N(OR^{k})C(=G)OR^{m}, C(=G)NR^{k}—NR^{m}Rⁿ, NR^{k}SO₂R^{m}, PR^{m}Rⁿ, SiR^{k}_{z}R^{m}_{3-z} with z being 0, 1, 2 or 3,
phenyl or a 3-, 4-, 5-, 6- or 7-membered heterocycle containing 1, 2, 3 or 4 heteroatoms selected from oxygen, nitrogen and sulfur, wherein the phenyl or the heterocycle may be unsubstituted or may carry 1, 2, 3, 4 or 5 radicals selected from halogen and C₁-C₄-alkyl;
G is oxygen or sulfur;
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, phenyl, benzyl or a 3-, 4-, 5-, 6- or 7-membered heterocycle containing 1, 2, 3 or 4 heteroatoms as ring members, selected from oxygen, nitrogen and sulfur, wherein C₃-C₈-cycloalkyl, C₃-C₈-cycloalkenyl, phenyl, benzyl and the heterocycle may be unsubstituted or may carry 1, 2, 3, 4 or 5 radicals selected from halogen and C₁-C₄-alkyl,
or R^{m} and Rⁿ together with the nitrogen, to which they are bound may form an N-bound 5-, 6- or 7-membered heterocycle, which in addition to the nitrogen atom may contain a further heteroatom selected from oxygen, nitrogen and sulphur,
R^{b} is halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, benzyl or a radical R^{a};
or, if X is not a chemical bond, R⁶ may also be a radical of the formulae OR^{c}, SR^{c}, S(═O)R^{c}, S(═O)₂R^{c}, NR^{d}R^{e}, N(OR^{c})R^{d}, S(═O)₂NR^{d}R^{e}, C(═O)R^{c}, C(═O)OR^{c}, O—C(═O)R^{c}, C(═O)NR^{d}R^{e}, C(═O)NR^{c}—NR^{d}R^{e}, C(═NOR^{c})R^{d}, C(═S)R^{c}, C(═S)OR^{c}, C(═S)NR^{d}R^{e}, NR^{d}C(=G)R^{c}, N(OR^{c})C(=G)R^{d}, N[C(=G)R^{c}][C(=G)R^{d}], NR^{d}C(=G)OR^{c}, N(OR^{c})C(=G)OR^{d}, NR^{k}SO₂R^{m}, SiR^{k}_{z}R^{m}_{3-z} with z being 0, 1, 2 or 3,
R^{c}, R^{d}, R^{e} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, phenyl, benzyl or a 3-, 4-, 5-, 6- or 7-membered heterocycle containing 1, 2, 3 or 4 heteroatoms a ring members, selected from oxygen, nitrogen and sulfur, wherein C₃-C₆-cycloalkyl, C₃-C₆-cycloalkenyl, phenyl, benzyl and the heterocycle may be unsubstituted or may carry 1, 2, 3, 4 or 5 radicals selected from halogen and C₁-C₄-alkyl,
or R^{d} and R^{e} together with the nitrogen, to which they are bound may form an N-bound 5-, 6- or 7-membered heterocycle, which in addition to the nitrogen atom may contain a further heteroatom selected from oxygen, nitrogen and sulphur;
R⁷ is hydrogen, C₁-C₆-alkyl, C₂-C₆-alkenyl, C₂-C₆-alkynyl, wherein the last three mentioned radicals may be unsubstituted or may carry 1, 2 or 3 identical or different radicals R^{f}, wherein R^{f} has the meanings given for R^{a},
C₁-C₆-haloalkyl, C₂-C₆-haloalkenyl, C₂-C₆-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from oxygen, nitrogen and sulphur, it being possible for the heterocycle to contain one carbonyl group as ring member, wherein C₃-C₆-cycloalkyl, C₃-C₆-cycloalkenyl, phenyl and the heterocycle may be unsubstituted or may carry 1, 2, 3, 4 or 5 substituents R^{g}, wherein R^{g} has the meanings given for R^{b},
wherein C₃-C₈-cycloalkyl, C₃-C₈-cycloalkenyl, phenyl and the heterocycle may also be fused to a ring selected from phenyl and a 5- to 6-membered saturated, partially unsaturated or aromatic heterocycle which may contain 1, 2 or 3 heteroatoms selected from oxygen, nitrogen and sulfur,
or a radical of the formulae OR^{c}, SR^{c}, S(═O)R^{c}, S(═O)₂R^{c}, NR^{d}R^{e}, N(OR^{c})R^{d}, S(═O)₂NR^{d}R^{e}, C(═O)R^{c}, C(═O)OR^{c}, C(═O)NR^{d}R^{e}, C(═O)NR^{c}—NR^{d}R^{e}, C(═NOR^{c})R^{d}, C(═S)R^{c}, C(═S)OR^{c}, C(═S)NR^{d}R^{e}, NR^{d}C(=G)R^{c}, N(OR^{c})C(=G)R^{d}, N[C(=G)R^{c}][C(=G)R^{d}], NR^{d}C(=G)OR^{c}, N(OR^{c})C(=G)OR^{d}, NR^{k}SO₂R^{m}, SiR^{k}_{z}R^{m}_{3-z}, wherein G, n, R^{c}, R^{d}, R^{e}, R^{k} and R^{m} are as defined above.

Accordingly, the present invention relates to a method of combating pest, which method comprises contacting said pest or their food supply, habitat, breeding ground or their locus with a 3-pyridyl compound of formula I or a salt thereof as defined above.

The compounds of formula I are novel, except for compounds I, in which X—R⁶ is CH₃, and except for

i. compounds of the formula I, wherein:
R¹ is fluoro; R², R³ and R⁴ are hydrogen; A is a chemical bond;
y and n are zero; and the moiety X—R⁶ is selected from imidazolidin-2,3-dion-1-ylmethyl (succinimidylmethyl), phthalimide-1-yl, 2-oxo-tetrahydrofuran-4-yl, 2-oxo-2,5-dihydrofuran-4-yl, N-(2-hydroxybenzoyl)aminomethyl, 5,5-dimethyl-3-oxocyclohexenyl, 3-oxocyclohexenyl, 2,2-bis(ethoxycarbonyl)ethenyl, ethyl, n-propyl, n-butyl, 2-propyl, n-pentyl, 2-methylpropan-1-yl, 3,3-dimethylpropin-3-yl and 1,1-dimethylpropan-1-yl;
   ii. the compound of the formula I, wherein:
R² is fluoro; R¹, R³ and R⁴ are hydrogen; A is a chemical bond; y and n are zero;
and the moiety X—R⁶ is n-propyl;
   iii. compounds of the formula I, wherein:
R¹ is chloro; R², R³ and R⁴ are hydrogen; A is a chemical bond; y and n are zero;
and the moiety X—R⁶ is ethyl, 3,3-dimethylpropin-3-yl or 1,1-dimethylpropan-1-yl;
   iv. the compounds of the formula I, wherein:
R¹ is methyl; R², R³ and R⁴ are hydrogen; A is a chemical bond; y and n are zero;
and the moiety X—R⁶ is ethyl;
   v. the compounds of the formula I, wherein:
R¹ is chloro; R², is methyl; R³ and R⁴ are hydrogen; A is a chemical bond; y and n are zero; and the moiety X—R⁶ is n-pentyl, 1,1-dimethylpropan-1-yl or 3,3-dimethylpropin-3-yl;
   vi. compounds of the formula I, wherein:
R¹, R², R³ and R⁴ are hydrogen, A is CH₂; y and n are zero; and the moiety X—R⁶ is selected from ethyl, 3-propenyl (allyl), 3-hydroxylpropan-1-yl, 3-fluoropropan-1-yl and 3-(methylsulfonyloxy)propan-1-yl;
   vii. the compound of the formula I, wherein:
R² is bromo; R¹, R³ and R⁴ are hydrogen; A is CH₂; y and n are zero; and the moiety X—R⁶ is 3-propenyl;
   viii. the compound of the formula I, wherein:
R¹ is chloro; R², R³ and R⁴ are hydrogen; A is a chemical bond;
y and n are zero; and the moiety X—R⁶ is 3-amino-4-[(2-chloropyridin-5-yl)oxy]-butyl.

Accordingly, the present invention also relates to novel 3-pyridyl compound of formula I and agriculturally acceptable salts thereof as defined in the preceding paragraphs.

Furthermore, the present invention relates to a method for protecting growing plants from attack or infestation by pests which method comprises applying to the plant, or to the soil or the water in which the plant is growing, at least one compound of formula I as defined above or a salt thereof. The present invention also relates to a method, wherein said compound of formula I or the salt thereof, respectively, is applied at a rate of 5 g/ha to 2000 g/ha. Furthermore, the present invention relates to a method for combating pests, wherein the pests are insects

Furthermore, the present invention relates to a method for protection of seed which comprises contacting the seeds with at least one compound of formulae (I) or an agriculturally acceptable salt thereof as defined herein. For seed protection, the at least one compound of formula (I) and/or a N-oxide or an agriculturally acceptable salt thereof is applied in an amount of from 0.1 g to 10 kg per 100 kg of seeds. The invention also relates to seed, comprising at least one compound of formula (I) and/or a N-oxide or an agriculturally acceptable salt thereof as defined herein.

Furthermore, the present invention also relates to a pesticidal composition, which comprises at least one compound of the formula I as defined above or a salt thereof and an agriculturally acceptable carrier. Preferably, said composition is formulated into dusting powders or granules, dispersible powders, granules or grains, aqueous dispersions, suspensions, pastes, or emulsions.

The compounds of the present invention are useful for combating pests. Accordingly, the present invention relates to the use of compounds of formula I for combating pests.

The present invention also relates to synergistic pesticidal mixtures, comprising a compound of formula I as defined in claims 1 to 16 and a pesticide selected from the organo(thio)phosphates, carbamates, pyrethroids, growth regulators, neonicotinoids, nicotinic receptor agonists/antagonists compounds, GABA antagonist compounds, macrocyclic lactone insecticides, METI I, II and III compounds, oxidative phosphorylation inhibitor compounds, moulting disruptor compounds, mixed function oxidase inhibitor compounds, sodium channel blocker compounds, benclothiaz, bifenazate, cartap, flonicamid, pyridalyl, pymetrozine, sulfur, thiocyclam, flubendiamide, cyenopyrafen, flupyrazofos, cyflumetofen, amidoflumet, N—R′-2,2-dihalo-1-R″-cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, and anthranilamide compounds of formula Γ³

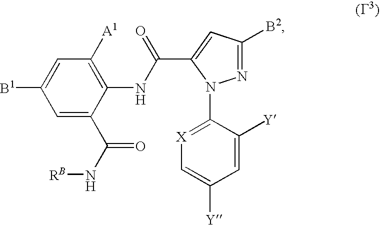

wherein A¹ is CH₃, Cl, Br, I, X is C—H, C—Cl, C—F or N, Y′ is F, Cl, or Br, Y″ is F, Cl, CF₃, B¹ is hydrogen, Cl, Br, I, CN, B² is Cl, Br, CF₃, OCH₂CF₃, OCF₂H, and R^{B} is hydrogen, CH₃ or CH(CH₃)₂.

### DETAILED DESCRIPTION OF THE INVENTION

Suitable compounds of the formula I encompass all possible stereoisomers (cis/trans isomers, enantiomers) which may occur and mixtures thereof. The present invention provides both the pure enantiomers or diastereomers or mixtures thereof, the pure cis- and trans-isomers and the mixtures thereof.

The invention comprises in particular the enantiomer I-R having R-configuration at the indicated carbon atom * and the enantiomer I-S having S-configuration at the indicated carbon atom * as well as mixtures thereof, including racemic mixtures and non-racemic mixtures, wherein one of the enantiomers is present in enantiomeric excess.

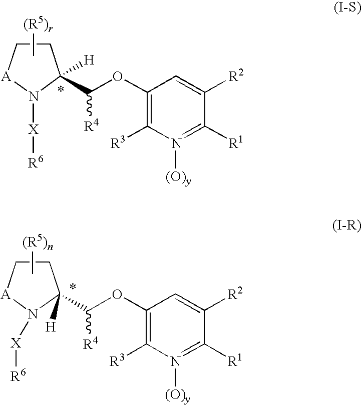

Salts of the compounds of the formulae I and II (and likewise of the pyridine N-oxides of I or II) are preferably agriculturally acceptable salts. They can be formed in a customary method, e.g. by reacting the compound with an acid of the anion in question if the compound of formulae I or II or the pyridine N-oxide thereof has a basic functionality or by reacting an acidic compound of formulae I or II with a suitable base.

Suitable agriculturally useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄_{⁺}) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium. Anions of useful acid addition salts are primarily chloride, bromide, fluoride, hydrogen sulphate, sulphate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formula I with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ—Cₘ indicates in each case the possible number of carbon atoms in the group.

“Halogen” will be taken to mean fluoro, chloro, bromo and iodo.

The term “C₁-C₁₀-alkyl” as used herein refers to a branched or non-branched (linear) saturated hydrocarbon group having 1 to 10 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl. C₁-C₄-alkyl means for example methyl, ethyl, propyl, 1-methylethyl, butyl, 1-methylpropyl, 2-methylpropyl or 1,1-dimethylethyl. Likewise, the terms “C₁-C₈-alkyl” “C₁-C₆-alkyl” and “C₁-C₄-alkyl” as used herein refers to a branched or non-branched (linear) saturated hydrocarbon group having 1 to 8, 1 to 6 or 1 to 4 carbon atoms, respectively.

The term “C₁-C₁₀-haloalkyl” as used herein refers to a straight-chained or branched alkyl group having 1 to 10 carbon atoms (as mentioned above), wherein some or all of the hydrogen atoms in these groups are replaced by halogen atoms, in particular fluorine or chlorine atoms, as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 5-fluoro-1-pentyl, 5-chloro-1-pentyl, 5-bromo-1-pentyl, 5-iodo-1-pentyl, 5,5,5-trichloro-1-pentyl, undecafluoropentyl, 6-fluoro-1-hexyl, 6-chloro-1-hexyl, 6-bromo-1-hexyl, 6-iodo-1-hexyl, 6,6,6-trichloro-1-hexyl or dodecafluorohexyl. Likewise, the terms “C₁-C₈-haloalkyl”, “C₁-C₆-haloalkyl” and “C₁-C₄-haloalkyl” as used herein refers to a branched or non-branched (linear) saturated hydrocarbon group having 1 to 8, 1 to 6 or 1 to 4 carbon atoms, respectively, wherein some or all of the hydrogen atoms in these groups are replaced by halogen atoms, in particular fluorine or chlorine atoms, as mentioned above. Likewise, the term “halomethyl” as used herein refers to methyl, wherein one, two or three hydrogen atoms are replaced by halogen atoms, in particular fluorine or chlorine atoms, for example chloromethyl, dichloromethyl, trichloromethyl, fluoromethyl, difluoromethyl and trifluoromethyl, especially fluoromethyl, difluoromethyl and trifluoromethyl.

Similarly, “C₁-C₆-alkoxy” and “C₁-C₆-alkylthio (or alkylsulfanyl, respectively)” refer to straight-chain or branched alkyl groups having 1 to 6 carbon atoms (as mentioned above) bonded through oxygen or sulfur linkages, respectively, at any bond in the alkyl group. Examples include C₁-C₄-alkoxy such as methoxy, ethoxy, propoxy, isopropoxy, butoxy, sec-butoxy, isobutoxy and tert-butoxy and C₁-C₄-alkylthio such as methylthio, ethylthio, propylthio, isopropylthio, and n-butylthio.

Similarly, “C₁-C₆-alkylsulfinyl” and “C₁-C₆-alkylsulfonyl” refer to straight-chain or branched alkyl groups having 1 to 6 carbon atoms (as mentioned above) bonded through —S(═O)— or —S(═O)₂-linkages, respectively, at any bond in the alkyl group. Examples include methylsulfinyl and methylsulfonyl.

Similarly, “C₁-C₆-haloalkoxy” and “C₁-C₆-haloalkylthio” (or haloalkylsulfanyl, respectively) refer to straight-chain or branched alkyl groups having 1 to 6 carbon atoms (as mentioned above) bonded through an oxygen atom or a sulfur atom, respectively, at any bond in the alkyl group, where some or all of the hydrogen atoms in these groups are replaced by halogen atoms, in particular fluorine or chlorine atoms, as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like.

Similarly, “C₁-C₆-alkylamino” and “C₁-C₆-dialkylamino” refer to a nitrogen atom which carries 1 or 2 straight-chain or branched alkyl groups having 1 to 6 carbon atoms (as mentioned above) which may be the same or different. Examples include methylamino, dimethylamino, ethylamino, diethylamino, methylethylamino, isopropylamino, or methylisopropylamino.

The terms “C₂-C₁₀-alkenyl” and “C₂-C₆-alkenyl” as used herein refer to a branched or non-branched unsaturated hydrocarbon group having 2 to 10 carbon atoms or 2 to 6 carbon atoms, respectively, and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The terms “C₂-C₁₀-haloalkenyl” and “C₂-C₆-haloalkenyl” as used herein refer to a branched or non-branched unsaturated hydrocarbon group having 2 to 10 carbon atoms or 2 to 6 carbon atoms, respectively, and a double bond in any position, where some or all of the hydrogen atoms in these groups are replaced by halogen atoms, in particular fluorine or chlorine atoms as mentioned above.

The terms “C₂-C₁₀-alkynyl” and “C₂-C₆-alkynyl” as used herein refer to a branched or non-branched unsaturated hydrocarbon group having 2 to 10 carbon atoms or 2 to 6 carbon atoms, respectively, and containing at least one triple bond, such as ethynyl, propynyl, 1-butynyl, 2-butynyl, and the like.

The terms “C₂-C₁₀-haloalkynyl” and “C₂-C₆-haloalkenyl” as used herein refer to a branched or non-branched unsaturated hydrocarbon group having 2 to 10 carbon atoms or 2 to 6 carbon atoms, respectively, and containing at least one triple bond, where some or all of the hydrogen atoms in these groups may be replaced by halogen atoms, in particular fluorine or chlorine atoms, as mentioned above.

The term “C₃-C₈-cycloalkyl” as used herein refers to a monocyclic 3- to 8-membered saturated carbon atom ring, e.g. cyclopropyl, cyclobutyl, cyclopentyl, cyclohexyl, cycloheptyl and cyclooctyl.

The term “C₀-C₆-alkylene” as used herein refers to a single bond or to a saturated bivalent hydrocarbon radical having 1 to 6 carbon atoms such as methylene, ethane-1,2-diyl, ethane-1,1-diyl, propane-1,1-diyl, propane-1,2-diyl, propane-1,3-diyl, propane-2,2-diyl, butane-1,4-diyl, pentan-1,5-diyland hexane-1,6-diyl.

The term “C₁-C₃-alkylene” as used herein refers to a saturated bivalent hydrocarbon radical having 1 to 3 carbon atoms such as methylene, ethane-1,2-diyl, ethane-1,1-diyl, propane-1,1-diyl, propane-1,2-diyl, propane-1,3-diyl, propane-2,2-diyl.

The term “phenyl-C₁-C₆-alkyl” as used herein refers to a phenyl radical which is attached to the remainder of the molecule by a C₁-C₆-alkylene group, e.g. benzyl, 1-phenylethan-1-yl, 2-phenylethan-1-yl, 2-methyl-2-phenylethan-1-yl and the like.

The term “3-, 4-, 5-, 6- or 7-membered heterocycle containing 1, 2, 3 or 4 heteroatoms selected from oxygen, nitrogen and sulphur” includes 3, 4, 5-, 6-, and 7-membered saturated, partially unsaturated or aromatic heterocycles which contain 1, 2, 3 or 4 heteroatoms as ring members, the heteroatoms being selected from the group consisting of oxygen, nitrogen and sulfur. The term includes in particular:

five- or six-membered saturated or partially unsaturated heterocycles (hereinbelow also referred to as heterocyclyl) which contain one, two, three or four heteroatoms from the group consisting of oxygen, nitrogen and sulfur as ring members: for example monocyclic saturated or partially unsaturated heterocycles which contain, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-dihydropyranyl, 3-dihydropyranyl, 4-dihydropyranyl, 2-thianyl, 3-thianyl, 4-thianyl (or tetrahydrothiopyranyl respectively), dehydrothianyl (or dihydrothiopyranyl respectively), 3,4-dihydro-2H-thiopyranyl, 3,6-dihydro-2H-thiopyranyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl, morpholin-2-yl, morpholin-3-yl, morpholin-4-yl;

seven-membered saturated or partially unsaturated heterocycles which contain one, two, three or four heteroatoms from the group consisting of oxygen, nitrogen and sulfur as ring members: for example mono- and bicyclic heterocycles having 7 ring members which contain, in addition to carbon ring members, one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals;

five- or six-membered aromatic heterocycles (=heteroaromatic radical or hetaryl, respectively) which contain one, two, three or four heteroatoms from the group consisting of oxygen, nitrogen and sulfur: mono- or bicyclic heteroaryl, for example 5-membered heteroaryl which is attached via carbon and contains one to three nitrogen atoms or one or two nitrogen atoms and one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl which contains one, two or three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Phenyl which is fused to phenyl or a 5- to 6-membered saturated, partially unsaturated or aromatic heterocyclic ring which may contain 1 to 3 heteroatoms selected from oxygen, nitrogen and sulfur is e.g. naphthalenyl, benzoxazolyl, benzthiazolyl, benzimidazolyl, benzoxadiazolyl, or benzthiadiazolyl.

In one embodiment of the compounds of formula I according to the present invention and their salts, the moiety X—R⁶ in formula I is different from C₂-C₆-alkyl or allyl, if the radicals R¹ and R² are selected from the group consisting of hydrogen, halogen, or methyl, and R⁴ is hydrogen, in particular if R¹ and R² are selected from the group consisting of hydrogen, halogen, OH, C₁-C₄-alkyl, CN, CH₂F, CHF₂, CF₃, NO₂, CH₂OH, CH₂CN, C₁-C₃-alkoxy, NH₂, NHCHO, NHC(O)—(C₁-C₃-alkyl), N(C₁-C₃-alkyl)C(O)—(C₁-C₃-alkyl), NH(C₁-C₃-alkyl), NCH₂-phenyl, N(C₁-C₃-alkyl)₂, C(O)OH, C(O)—O—C₁-C₃-alkyl, C(O)NH₂, C(O)—NH—C₁-C₃-alkyl, C(O)—NH—CH₂-phenyl and O—C(O)—(C₁-C₃-alkyl), and R⁴ is hydrogen.

In another embodiment of the compounds of formula I according to the present invention and their salts, the moiety X—R⁶ is different from 3-amino-4-[(2-chloropyridin-5-yl)oxy]butyl, 3-hydroxylpropan-1-yl, 3-fluoropropan-1-yl, 3-(methylsulfonyloxy)propan-1-yl, 3,3-dimethylpropin-3-yl, imidazolidin-2,3-dion-1-ylmethyl (succinimidylmethyl), phthalimide-1-yl, 2-oxo-tetrahydrofuran-4-yl, 2-oxo-2,5-dihydrofuran-4-yl, N-(2-hydroxybenzoyl)aminomethyl, 5,5-dimethyl-3-oxocyclohexenyl, 3-oxocyclohexenyl, or 2,2-bis(ethoxycarbonyl)ethenyl, if R¹ and R² are selected from hydrogen, halogen and methyl, and R⁴ is hydrogen, in particular, if R¹ and R² are selected from the group consisting of halogen, OH, C₁-C₄-alkyl, CN, CH₂F, CHF₂, CF₃, NO₂, CH₂OH, CH₂CN, C₁-C₃-alkoxy, NH₂, NHCHO, NHC(O)—(C₁-C₃-alkyl), N(C₁-C₃-alkyl)C(O)—(C₁-C₃-alkyl), NH(C₁-C₃-alkyl), NCH₂-phenyl, N(C₁-C₃-alkyl)₂, C(O)OH, C(O)—O—C₁-C₃-alkyl, C(O)NH₂, C(O)—NH—C₁-C₃-alkyl, C(O)—NH—CH₂-phenyl and O—C(O)—(C₁-C₃-alkyl), and R⁴ is hydrogen.

In another embodiment of the compounds of formula I according to the present invention and their salts, the moiety X—R⁶ in formula I is different from C₂-C₆-alkyl or allyl, if

R¹ is hydrogen, alkyl, fluorine, chlorine, phenyl or ethenyl,

R² is a radical L-R^{z}, wherein L is selected from a chemical bond, —C═C—(C₀-C₆-alkylene)-, —(CH═CH)ₚ—(C₀-C₆-alkylene)- with p being 1 or 2, —CH═CH—C(O)—(C₀-C₆-alkylene)-, —C(O)—(C₀-C₆-alkylene)- and —CH₂-M-C(O)—(C₀-C₆-alkylene)- with M being CH₂ or NH and R^{z} is selected from hydrogen, optionally substituted C₁-C₈-alkyl, C₁-C₆-haloalkyl, C₁-C₆-hydroxyalkyl, C₁-C₆-alkoxy, amino, C₁-C₆-alkylamino, C₁-C₆-dialkylamino, an azacycle attached to L through a nitrogen atom, phenyl, naphthyl, biphenyl, furyl, thienyl, pyridinyl, pyridazinyl, pyrimidinyl, pyrrolyl, pyrazolyl, imidazolyl, indolyl, thiazolyl, oxazolyl, isoxazolyl, thiadiazolyl, oxadiazolyl, quinolinyl, and isoquinolyl, wherein alkyl and the 19 last mentioned radicals may carry one or two substituents, selected from C₁-C₆-alkyl, C₁-C₆-haloalkyl, C₁-C₆-alkoxy-C₁-C₆-alkyl, C₁-C₆-alkoxy, C₁-C₆-alkoxy-C₁-C₆-alkoxy, halogen, cyano, hydroxy, amino, C₁-C₆-alkylamino, carboxyl and C₁-C₆-alkoxycarbonyl; and

R⁴ is hydrogen.

In particular, those compounds of the formula I and their salts are not part of the invention, where the moiety X—R⁶ in formula I is different from C₂-C₆-alkyl or allyl, 3-amino-4-[(2-chloropyridin-5-yl)oxy]butyl, 3-hydroxylpropan-1-yl, 3-fluoropropan-1-yl, 3-(methylsulfonyloxy)propan-1-yl, 3,3-dimethylpropin-3-yl, imidazolidin-2,3-dion-1-ylmethyl (succinimidylmethyl), phthalimide-1-yl, 2-oxo-tetrahydrofuran-4-yl, 2-oxo-2,5-dihydrofuran-4-yl, N-(2-hydroxybenzoyl)aminomethyl, 5,5-dimethyl-3-oxocyclohexenyl, 3-oxocyclohexenyl, or 2,2-bis(ethoxycarbonyl)ethenyl, and R⁴ is hydrogen.

In a further embodiment of the compounds of formula I according to the present invention and their salts, the moiety X—R⁶ in formula I is different from C₂-C₆-alkyl, C₂-C₆-alkenyl, C₂-C₆-alkynyl, C₃-C₇-cycloalkyl or C₃-C₇-cycloalkenyl, wherein the last 5 mentioned radicals may carry at least one substituent selected from halogen, hydroxyl, carboxy, cyano, nitro, sulfonamide, sulfonate, phosphate, amino, C₁-C₄-alkylamino or dialkylamino, if one of the radicals R¹ and R² is hydrogen, while the other radical is selected from the group consisting halogen, amino, C₁-C₆-alkyl, C₁-C₆-alkoxy, C₂-C₆-alkenyl, C₂-C₆-alkynyl, C₃-C₇-cycloalkyl or C₃-C₇-cycloalkenyl, wherein the last 5 mentioned radicals may carry at least one substituent selected from halogen, hydroxyl, carboxy, cyano, nitro, sulfonamide, sulfonate, phosphate, amino, C₁-C₄-alkylamino or dialkylamino, and R⁴ is hydrogen.

With respect to the intended use of the compounds of the formula I, particular preference is given to compounds of the formula I, wherein the variables y, n, A, X, R¹, R², R³, R⁴, R⁵ and R⁶ independently of each other, and in particular in combination, have one of the following meanings:

y is 0.

n is 0 or 1, in particular 0.

X is a radical CHR⁷ or CHR⁷—CH₂. In the group CHR⁷—CH₂ preferably the CHR⁷-moiety is attached to R⁶. More preferably X is CHR⁷, in particular R². R⁷ is preferably selected from hydrogen, alkyl, in particular methyl, and a radical C≡C—R^{f}, wherein R^{f} is as defined herein, preferably hydrogen, C₁-C₄-alkyl, tri(C₁-C₄-alkyl)silyl, C₁-C₄-hydroxyalkyl or C₁-C₄-alkoxy-C₁-C₄-alkyl. In another preferred embodiment, X in formula I is a chemical bond.

A is CH₂ or a chemical bond, with preference given to the latter.

R¹ is selected from the group consisting of hydrogen, halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₈-halocycloalkenyl, C₁-C₆-alkoxy, C₁-C₆-haloalkoxy, C₁-C₆-alkylthio, C₁-C₆-alkylsulfinyl, C₁-C₆-haloalkylsulfinyl, C₁-C₆-alkylsulfonyl, C₁-C₆-haloalkylsulfonyl, di(C₁-C₆-alkyl)amino, C₁-C₆-alkylamino, C₁-C₆-alkoxycarbonyl, phenyl or 5- to 6-membered hetaryl which contains 1, 2, 3 or 4 heteroatoms selected from oxygen, nitrogen and sulfur, wherein the carbon atoms in phenyl and hetaryl may be substituted with 1, 2 or 3 groups selected from halogen, amino, cyano, C₁-C₆-alkyl, C₁-C₆-alkoxy, C₁-C₆-haloalkyl, C₁-C₆-haloalkoxy, C₁-C₆-alkylthio, C₁-C₆-haloalkylthio and nitro. Preferably, R¹ is selected from the group consisting of hydrogen, halogen, CN, NH₂, C₁-C₄-alkyl, which may be substituted by one OH or CN radical, C₁-C₄-haloalkyl, NHCHO and NHC(O)—C₁-C₄-alkyl, in particular from the group consisting of hydrogen, methyl, CN, CHF₂, CHF₂, CF₃, CH₂OH, CH₂CN, NH₂, NHCHO or halogen and more preferably hydrogen or halogen.

R² is selected from the group consisting of hydrogen, halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₈-halocycloalkenyl, C₁-C₆-alkoxy, C₁-C₆-haloalkoxy, C₁-C₆-alkylthio, C₁-C₆-alkylsulfinyl, C₁-C₆-haloalkylsulfinyl, C₁-C₆-alkylsulfonyl, C₁-C₆-haloalkylsulfonyl, di(C₁-C₆-alkyl)amino, C₁-C₆-alkylamino, C₁-C₆-alkoxycarbonyl, phenyl or 5- to 6-membered hetaryl which contains 1, 2, 3 or 4 heteroatoms selected from oxygen, nitrogen and sulfur, wherein the carbon atoms in phenyl and hetaryl may be substituted with 1, 2 or 3 groups selected from halogen, amino, cyano, C₁-C₆-alkyl, C₁-C₆-alkoxy, C₁-C₆-haloalkyl, C₁-C₆-haloalkoxy, C₁-C₆-alkylthio, C₁-C₆-haloalkylthio and nitro. Preferably, R² is selected from the group consisting of hydrogen, halogen, CN, NH₂, C₁-C₆-alkyl, which may be substituted by one OH, phenyl or CN radical, C₁-C₆-haloalkyl, C₂-C₆-alkenyl, C₂-C₆-alkynyl and NHC(O)—C₁-C₆-alkyl.

Likewise, preference is given to compounds of the formula I, wherein R² is Z—R^{y}, wherein Z is selected from

—C≡C—(C₀-C₆-alkylene)-, —(CH═CH)ₚ—(C₀-C₆-alkylene)- with p being 1 or 2, —CH═CH—C(O)—(C₀-C₆-alkylene)-, —C(O)—(C₀-C₆-alkylene)- and —CH₂—Z′—C(O)—(C₀-C₆-alkylene)- with Z' being CH₂ or NH and R^{y} is selected from hydrogen, C₁-C₈-alkyl, C₁-C₆-haloalkyl, C₁-C₆-hydroxyalkyl, C₁-C₆-alkoxy, amino, C₁-C₆-alkylamino, C₁-C₆-dialkylamino, 5 to 7 membered, azaheterocyclyl, such as pyrrolidinyl, piperidinyl, morpholinyl, piperazinyl or thiomorpholinyl, attached to Z via its nitrogen atom, phenyl, naphthyl, biphenyl, furyl, thienyl, pyridinyl, pyridazinyl, pyrimidinyl, pyrrolyl, pyrazolyl, imidazolyl, indolyl, thiazolyl, oxazolyl, isoxazolyl, thiadiazolyl, oxadiazolyl, quinolinyl, and isoquinolyl, wherein alkyl and the 19 last mentioned radicals may carry one or two substituents, selected from C₁-C₆-alkyl, C₁-C₆-haloalkyl, C₁-C₆-alkoxy-C₁-C₆-alkyl, C₁-C₆-alkoxy, C₁-C₆-alkoxy-C₁-C₆-alkoxy, halogen, cyano, hydroxy, amino, C₁-C₆-alkylamino, carboxyl and C₁-C₆-alkoxycarbonyl.

Preferably, at least one of the radicals R¹ and R² are different from hydrogen. A very preferred embodiment of the invention relates to compounds of the formula I, wherein R¹ and R² are each independently selected from halogen and hydrogen, with most preference given to those compounds, wherein at least one of the radicals R¹ and R² are different from hydrogen.

R³ is hydrogen, fluoro or methyl, in particular hydrogen.

R⁴ is hydrogen or methyl, with preference given to hydrogen.

R⁵, if present, is preferably selected from methyl, OH, methoxy fluoro, chloro, and cyano.

Particular preference to the compounds of the following formulae I-1 and I-2, wherein R¹, R², X and R⁶ are as defined above.

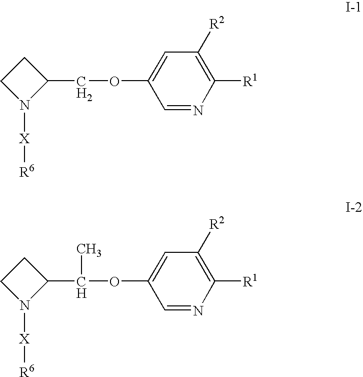

Likewise, preference to the compounds of the following formulae I-3 and I-4, wherein R¹, R², X and R⁶ are as defined above.

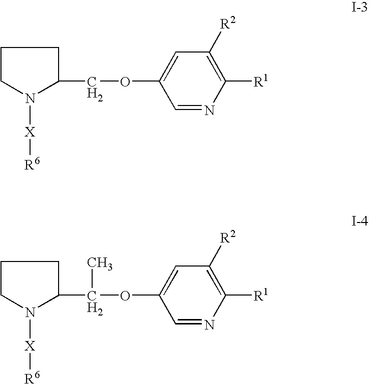

A very preferred embodiment of the invention relates to compounds of the formula I, and likewise to the compounds of the formulae I-1, I-2, I-3 and I-4, wherein R⁶ is C₃-C₈-cycloalkyl, C₃-C₈-cycloalkenyl, phenyl or a 3-, 4-, 5-, 6- or 7-membered heterocycle containing 1, 2, 3 or 4 heteroatoms selected from oxygen, nitrogen and sulfur, wherein C₃-C₈-cycloalkyl, C₃-C₈-cycloalkenyl, phenyl and the heterocycle may be unsubstituted or may carry 1, 2, 3, 4 or 5 substituents R^{b}, it being also possible for C₃-C₈-cycloalkyl, C₃-C₈-cycloalkenyl, phenyl and the heterocycle to be fused to 1 or 2 rings selected from phenyl and a 5- to 6-membered saturated, partially unsaturated or aromatic heterocycle which may contain 1, 2 or 3 heteroatoms selected from oxygen, nitrogen and sulphur.

Amongst these compounds, preference is given to those, wherein R⁶ is phenyl, that is unsubstituted or carries 1, 2 or 3 substituents R^{b} as defined herein.

Amongst these compounds, likewise preference is given to those compounds, wherein R⁶ is 5- or 6 membered hetaryl, in particular pyrrolyl, furyl, thienyl, imidazolyl, pyrazolyl, isoxazolyl, isothiazolyl, oxazolyl, thiazolyl or pyridyl, wherein hetaryl is unsubstituted or carries 1, 2 or 3 substituents R^{b} as defined herein. Particular preferred hetaryl is 2-pyridyl, 3-pyridyl, 4-pyridyl, 2-thienyl, 3-thienyl, 2-furyl, 3-furyl, 3-isothiazolyl, 4-isothiazolyl, 5-isothiazolyl, 3-isoxazolyl, 4-isoxazolyl, 5-isoxazolyl, 2-oxazolyl, 4-oxazolyl, 5-oxazolyl, 2-thiazolyl, 4-thiazolyl, 5-thiazolyl, wherein hetaryl is unsubstituted or carries 1, 2 or 3 substituents R^{b} as defined herein.

R⁷, if present, is preferably selected from hydrogen, C₁-C₆-alkyl, C₂-C₆-alkenyl, C₂-C₆-alkynyl, wherein the last three mentioned radicals may be unsubstituted or may carry 1, 2 or 3 identical or different radicals R^{f} as defined herein.

Apart from that, the radicals G, R^{a}, R^{b}, R^{c}, R^{d}, R^{e}, R^{f}, Rⁱ, R^{j}, R^{k}, R^{m} and Rⁿ preferably have one of the following meanings:

G is oxygen.

R^{a} is cyano, nitro, C₃-C₈-cycloalkenyl, C₃-C₈-halocycloalkenyl, OR^{k}, SR^{k}, S(═O)₂R^{k}, NR^{m}Rⁿ, N(OR^{k})R^{m}, S(═O)₂NR^{m}Rⁿ, O—C(═O)R^{k}, C(═O)R^{k}, C(═O)OR^{k}, C(═O)NR^{m}Rⁿ, C(═NOR^{k})R^{m}, NR^{k}C(═O)R^{m}, NR^{k}C(═O)OR^{m}, C(═O)NR^{k}—NR^{m}Rⁿ, NR^{k}SO₂R^{m}, wherein R^{k}, R^{m} and Rⁿ are as defined herein. In particular R^{a} is selected from OH, OR^{k}, SR^{k}, NH₂ and NHC(O)R^{k} and OC(O)R^{k} with R^{k} being hydrogen, C₁-C₄-alkyl, C₁-C₄-haloalkyl, phenyl or pyridyl it being possibly for phenyl and pyridyl to carry 1 or 2 substituents, selected from halogen and C₁-C₄-alkyl.

R^{b} is halogen, cyano, nitro, C₁-C₆-alkyl, C₁-C₆-haloalkyl, C₃-C₆-cycloalkenyl, C₃-C₆-halocycloalkenyl, C₂-C₆-alkenyl, benzyl, phenoxy or benzyloxy, wherein the phenyl ring in the four last mentioned radicals may be unsubstituted or may carry 1, 2 or 3 halogen atoms, OR^{k}, SR^{k}, S(═O)₂R^{k}, NR^{m}Rⁿ, N(OR^{k})R^{m}, S(═O)₂NR^{m}Rⁿ, O—C(═O)R^{k}, C(═O)R^{k}, C(═O)OR^{k}, C(═O)NR^{m}Rⁿ, C(═NOR^{k})R^{m}, NR^{k}C(═O)R^{m}, NR^{k}C(═O)OR^{m}, C(═O)NR^{k}—NR^{m}Rⁿ, NR^{k}SO₂R^{m}, wherein R^{k}, R^{m} and Rⁿ are as defined herein. In particular, R^{b} is selected from halogen, CN, OH, C₁-C₄-alkyl, C₁-C₄-haloalkyl, C₁-C₄-alkoxy, C₁-C₄-haloalkoxy, C₂-C₄-alkenyl, benzyl, phenoxy or benzyloxy, wherein the phenyl ring in the four last mentioned radicals may be unsubstituted or may carry 1, 2 or 3 halogen atoms, SR^{k}, S(O)₂R^{k}, NR^{k}R^{m}, C(O)R^{k}, CO(O)R^{k}, NHC(O)R^{k} and OC(O)R^{k} with R^{k} being hydrogen, C₁-C₄-alkyl, C₁-C₄-haloalkyl and phenyl, which may be unsubstituted or may carry 1, 2 or 3 halogen atoms.

R^{c} is hydrogen, C₁-C₄-alkyl, C₁-C₄-haloalkyl or phenyl, which may be unsubstituted or may carry 1, 2 or 3 halogen atoms.

R^{d} is hydrogen, C₁-C₄-alkyl, phenyl or benzyl, wherein the phenyl ring in the last 2 mentioned radicals may be unsubstituted or may carry 1, 2 or 3 halogen atoms.

R^{e} is hydrogen, C₁-C₄-alkyl or phenyl, which may be unsubstituted or may carry 1, 2 or 3 halogen atoms.

R^{f} is cyano, nitro, C₃-C₈-cycloalkenyl, C₃-C₈-halocycloalkenyl, OR^{k}, SR^{k}, S(═O)₂R^{k}, NR^{m}Rⁿ, N(OR^{k})R^{m}, S(═O)₂NR^{m}Rⁿ, O—C(═O)R^{k}, C(═O)R^{k}, C(═O)OR^{k}, C(═O)NR^{m}Rⁿ, C(═NOR^{k})R^{m}, NR^{k}C(═O)R^{m}, NR^{k}C(═O)OR^{m}, C(═O)NR^{k}—NR^{m}Rⁿ, NR^{k}SO₂R^{m}, or SiR^{k}_{z}R^{m}_{z-3}, wherein z, R^{k}, R^{m} and Rⁿ are as defined herein. In particular R^{f} is selected from OH, OR^{k}, SR^{k}, NH₂ and NHC(O)R^{k} and OC(O)R^{k} with R^{k} being hydrogen, C₁-C₄-alkyl, C₁-C₄-haloalkyl, phenyl or pyridyl it being possibly for phenyl and pyridyl to carry 1 or 2 substituents, selected from halogen and C₁-C₄-alkyl.

R^{g} is halogen, cyano, nitro, C₁-C₆-alkyl, C₁-C₆-haloalkyl, C₃-C₆-cycloalkenyl, C₃-C₆-halocycloalkenyl, C₂-C₆-alkenyl, benzyl, phenoxy or benzyloxy, wherein the phenyl ring in the four last mentioned radicals may be unsubstituted or may carry 1, 2 or 3 halogen atoms, OR^{k}, SR^{k}, S(═O)₂R^{k}, NR^{m}Rⁿ, N(OR^{k})R^{m}, S(═O)₂NR^{m}Rⁿ, O—C(═O)R^{k}, C(═O)R^{k}, C(═O)OR^{k}, C(═O)NR^{m}Rⁿ, C(═NOR^{k})R^{m}, NR^{k}C(═O)R^{m}, NR^{k}C(═O)OR^{m}, C(═O)NR^{k}—NR^{m}Rⁿ, NR^{k}SO₂R^{m}, wherein R^{k}, R^{m} and Rⁿ are as defined herein. In particular, R^{g} is selected from halogen, CN, OH, C₁-C₄-alkyl, C₁-C₄-haloalkyl, C₁-C₄-alkoxy, C₁-C₄-haloalkoxy, C₂-C₄-alkenyl, benzyl, phenoxy or benzyloxy, wherein the phenyl ring in the four last mentioned radicals may be unsubstituted or may carry 1, 2 or 3 halogen atoms, SR^{k}, S(O)₂R^{k}, NR^{k}R^{m}, C(O)R^{k}, CO(O)R^{k}, NHC(O)R^{k} and OC(O)R^{k} with R^{k} being hydrogen, C₁-C₄-alkyl, C₁-C₄-haloalkyl and phenyl, which may be unsubstituted or may carry 1, 2 or 3 halogen atoms.

Rⁱ is hydrogen, C₁-C₄-alkyl, C₁-C₄-haloalkyl or phenyl, which may be unsubstituted or may carry 1, 2 or 3 substituents selected from C₁-C₄-alkyl and halogen atoms.

R^{j} is hydrogen, C₁-C₄-alkyl or phenyl, which may be unsubstituted or may carry 1, 2 or 3 halogen atoms.

R^{k} is hydrogen, C₁-C₄-alkyl, C₁-C₄-haloalkyl or phenyl, which may be unsubstituted or may carry 1, 2 or 3 substituents selected from C₁-C₄-alkyl and halogen atoms.

R^{m} is hydrogen, C₁-C₄-alkyl or phenyl, which may be unsubstituted or may carry 1, 2 or 3 halogen atoms.

Rⁿ is hydrogen, C₁-C₄-alkyl, phenyl or benzyl, wherein the phenyl ring in the last 2 mentioned radicals may be unsubstituted or may carry 1, 2 or 3 halogen atoms.

A very preferred embodiment of the present invention relates to compounds of the formula I, in particular to compounds of the formulae I-1, I-2, I-3 and I-4, wherein R¹, R², R³, R⁴, A, n, y and R⁵ are as defined above, wherein

X is a radical CHR⁷ or CHR⁷—CH₂, in particular a radical CHR⁷, wherein R⁷ is as defined herein and wherein R⁷ is preferably selected from hydrogen, C₁-C₆-alkyl, C₂-C₆-alkenyl, C₂-C₆-alkynyl, wherein the last three mentioned radicals may be unsubstituted or may carry 1, 2 or 3 identical or different radicals R^{f} as defined, and wherein R⁷ is more preferably selected from hydrogen, C₁-C₄-alkyl, in particular methyl, and a radical C≡C—R^{f}, wherein R^{f} is as defined herein, preferably hydrogen, C₁-C₄-alkyl, tri(C₁-C₄-alkyl)silyl, C₁-C₄-hydroxyalkyl or C₁-C₄-alkoxy-C₁-C₄-alkyl and wherein

R⁶ is C₃-C₈-cycloalkyl, C₃-C₈-cycloalkenyl, phenyl or a 3-, 4-, 5-, 6- or 7-membered heterocycle containing 1, 2, 3 or 4 heteroatoms selected from oxygen, nitrogen and sulfur, wherein C₃-C₆-cycloalkyl, C₃-C₆-cycloalkenyl, phenyl and the heterocycle may be unsubstituted or may carry 1, 2, 3, 4 or 5 substituents R^{b}, it being also possible for C₃-C₈-cycloalkyl, C₃-C₈-cycloalkenyl, phenyl and the heterocycle to be fused to 1 or 2 rings selected from phenyl and a 5- to 6-membered saturated, partially unsaturated or aromatic heterocycle which may contain 1, 2 or 3 heteroatoms selected from oxygen, nitrogen and sulphur. In this embodiment R⁶ is particularly preferably phenyl or 5- or 6-membered hetaryl, wherein phenyl and hetaryl are unsubstituted or carry 1, 2 or 3 substituents R^{b} as defined above. In this embodiment R⁶ is more preferably phenyl or 5- or 6-membered hetaryl, which is unsubstituted or carries 1, 2 or 3 substituents R^{b} as defined above.

A further preferred embodiment of the present invention relates to compounds of the formula I, in particular to compounds of the formulae I-1, I-2, I-3 and I-4, wherein R¹, R², R³, R⁴, A, n, y and R⁵ are as defined above, wherein X is CHR⁷ and wherein R⁶ is selected from

C₁-C₈-alkyl, which may carry 1 or 2 radicals R^{a} as defined above, in particular radicals R^{a} selected from halogen, OH, OR^{k}, SR^{k}, NH₂ and NHC(O)R^{k} and OC(O)R^{k} with R^{k} being hydrogen, C₁-C₄-alkyl, C₁-C₄-haloalkyl, phenyl or pyridyl it being possibly for phenyl and pyridyl to carry 1 or 2 substituents, selected from halogen and C₁-C₄-alkyl;

C₃-C₆-cycloalkyl,

saturated or partially unsaturated 5- or 6-membered heterocyclyl, which is preferably selected from tetrahydrofuryl, dihydrofuryl, dihydropyranyl, tetrahydropyranyl, thianyl, dehydrothianyl, thiolanyl and morpholinyl;

5- or 6-membered hetaryl, which is preferably selected from pyrrolyl, furyl, thienyl, imidazolyl, pyrazolyl, isoxazolyl, isothiazolyl, oxazolyl, thiazolyl and pyridyl, it being possible for hetaryl to carry 1 or 2 radicals, selected from halogen, C₁-C₆-alkyl, phenyl, benzyl, phenoxy or benzyloxy, wherein the phenyl ring in the four last mentioned radicals may be unsubstituted or may carry 1, 2 or 3 halogen atoms;

phenyl, which may carry 1, 2 or 3 radicals R^{b} as defined above, wherein R^{b} is preferably selected from halogen, CN, OH, C₁-C₄-alkyl, C₁-C₄-haloalkyl, C₁-C₄-alkoxy, C₁-C₄-haloalkoxy, C₂-C₄-alkenyl, benzyl, phenoxy or benzyloxy, wherein the phenyl ring in the four last mentioned radicals may be unsubstituted or may carry 1, 2 or 3 halogen atoms, SR^{k}, S(O)₂R^{k}, NR^{k}R^{m}, C(O)R^{k}, CO(O)R^{k}, NHC(O)R^{k} and OC(O)R^{k} with R^{k} being hydrogen, C₁-C₄-alkyl, C₁-C₄-haloalkyl and phenyl, wherein the phenyl ring in the four last mentioned radicals may be unsubstituted or may carry 1, 2 or 3 halogen atoms;

and wherein R⁷ is C≡C—R^{f}, wherein R^{f} is as defined above.

A further preferred embodiment of the present invention relates to compounds of the formula I, in particular to compounds of the formulae I-1, I-2, I-3 and I-4, wherein R¹, R², R³, R⁴, A, n, y and R⁵ are as defined above, and wherein X—R⁶ is
C₁-C₁₀-alkyl, C₂-C₁₀-alkenyl, C₂-C₁₀-alkynyl, wherein the last three mentioned radicals may be unsubstituted or may carry 1, 2 or 3 identical or different radicals R^{a} as defined herein;
C₁-C₁₀-haloalkyl, C₂-C₁₀-haloalkenyl, C₂-C₁₀-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from oxygen, nitrogen and sulfur, wherein C₃-C₈-cycloalkyl, C₃-C₈-cycloalkenyl, phenyl and the heterocycle may be unsubstituted or may carry 1, 2, 3, 4 or 5 substituents R^{b}, as defined above.

A further preferred embodiment of the present invention relates to compounds of the formula I, in particular to compounds of the formulae I-1, I-2, I-3 and I-4, wherein R¹, R², R³, R⁴, A, n, y and R⁵ are as defined above, and wherein X—R⁶ is a radical of the formula:

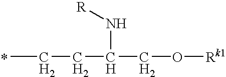

wherein * denotes the bonding position to the nitrogen of the heterocycle, R is hydrogen or a radical C(O)R^{k} and R^{k1} has one of the meanings given for R^{k} and wherein R^{k} is as defined above.

Particular preference is given to the compounds of the following formulae I-1.a, I-1.b, I-1.c, I-1.d, I-2.a, I-2.b, I-2.c and I-2.d, wherein R¹, R² and R⁶ are as defined above.

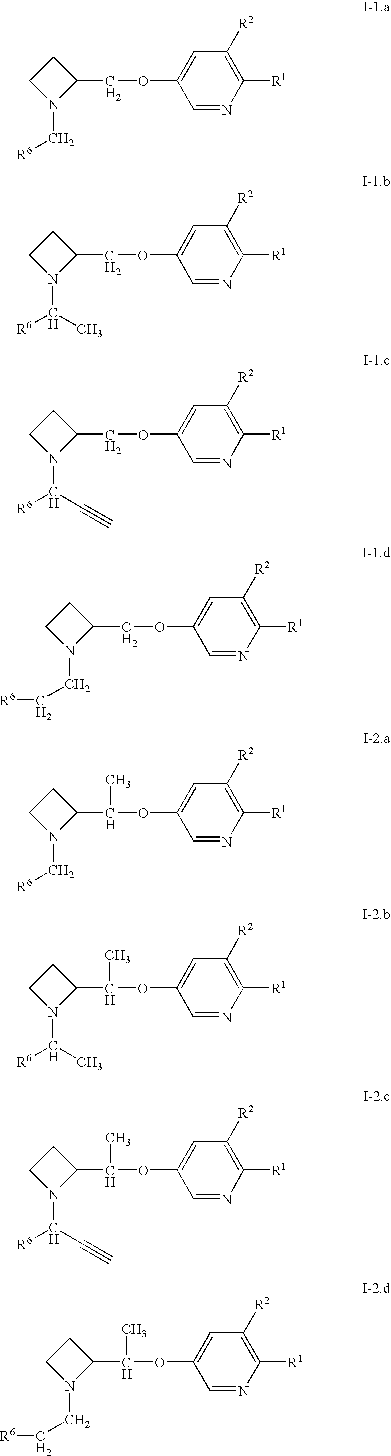

Likewise, preference is given to the compounds of the following formulae I-3.a, I-3.b, I-3.c, I-3.d, I-4.a, I-4.b, I-4.c and I-4.d, wherein R¹, R² and R⁶ are as defined above.

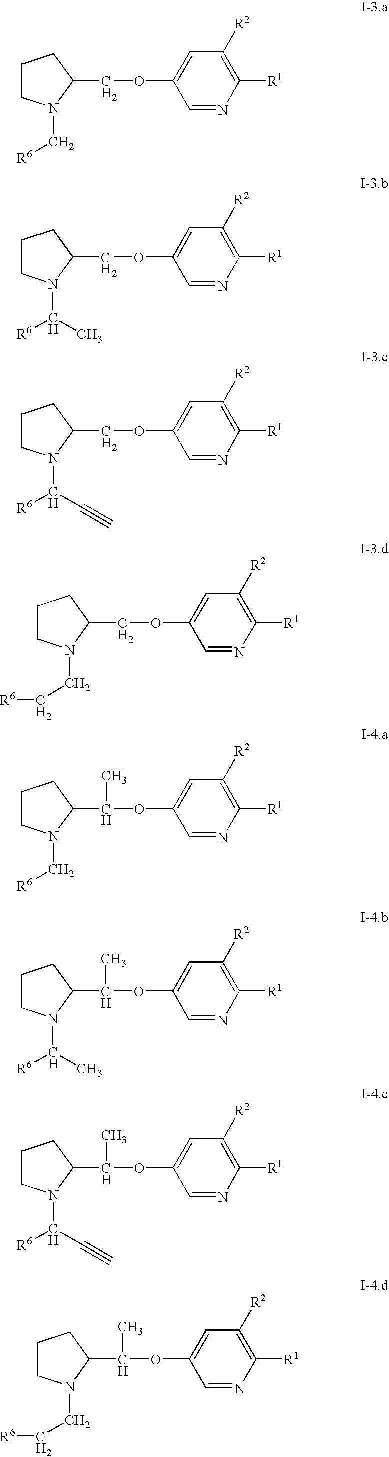

A particularly preferred embodiment of the present invention relates to compounds of the formula I, in particular to compounds of the formulae I-1, I-2, I-3 and I-4, more preferably compounds of the formulae I-1.a, I-1.b, I-1.c, I-1.d, I-2.a, I-2.b, I-2.c, I-2.d, I-3.a, I-3.b, I-3.c, I-3.d, I-4.a, I-4.b, I-4.c and I-4.d, wherein n and y are 0, R³ is hydrogen, A, R¹, R² and R⁴ are as defined above and R⁶ is 5- or 6-membered hetaryl as defined above, which is unsubstituted or substituted and which is particularly selected from a ring system Q of table A below.

TABLE A

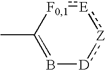

| No. | B | D | Z | E | F |
|---|---|---|---|---|---|
| Q.1 | ═N— | —C(R_{q})═ | ═C(R_{q})— | —C(R_{q})═ | ═C(R_{q})— |
| Q.2 | ═C(R_{q})— | —N═ | ═C(R_{q})— | —C(R_{q})═ | ═C(R_{q})— |
| Q.3 | ═C(R_{q})— | —C(R_{q})═ | ═N— | —C(R_{q})═ | ═C(R_{q})— |
| Q.4 | ═C(R_{q})— | —C(R_{q})═ | ═C(R_{q})— | —S— | — |
| Q.5 | ═C(R_{q})— | —S— | —C(R_{q})═ | ═C(R_{q})— | — |
| Q.6 | ═C(R_{q})— | —C(R_{q})═ | ═C(R_{q})— | —O— | — |
| Q.7 | ═C(R_{q})— | —O— | —C(R_{q})═ | ═C(R_{q})— | — |
| Q.8 | ═N— | —C(R_{q})═ | ═C(R_{q})— | —S— | — |
| Q.9 | ═N— | —C(R_{q})═ | ═C(R_{q})— | —O— | — |
| Q.10 | ═C(R_{q})— | —N═ | ═C(R_{q})— | —S— | — |
| Q.11 | ═C(R_{q})— | —N═ | ═C(R_{q})— | —O— | — |
| Q.12 | ═C(R_{q})— | —S— | —C(R_{q})═ | ═N— | — |
| Q.13 | ═C(R_{q})— | —O— | —C(R_{q})═ | ═N— | — |
| Q.14 | ═N— | —S— | —C(R_{q})═ | ═C(R_{q})— | — |
| Q.15 | ═N— | —O— | —C(R_{q})═ | ═C(R_{q})— | — |
| Q.16 | ═N— | —N(R_{q})— | —C(R_{q})═ | ═C(R_{q})— | — |
| Q.17 | ═N— | —N═ | ═C(R_{q})— | —N(R_{q})— | — |
| Q.18 | ═N— | —N═ | ═C(R_{q})— | —S— | — |
| Q.19 | ═N— | —N═ | ═C(R_{q})— | —O— | — |
| Q.20 | ═N— | —O— | —C(R_{q})═ | ═N— | — |
| Q.21 | ═N— | —N═ | ═C(R_{q})— | —C(R_{q})═ | ═C(R_{q})— |
| Q.22 | ═C(R_{q})— | —N═ | ═N— | —C(R_{q})═ | ═C(R_{q})— |
| Q.23 | ═N— | —C(R_{q})═ | ═N— | —C(R_{q})═ | ═C(R_{q})— |
| Q.24 | ═N— | —C(R_{q})═ | ═C(R_{q})— | —N═ | ═C(R_{q})— |
| Q.25 | ═N— | —C(R_{q})═ | ═C(R_{q})— | —C(R_{q})═ | ═N— |
| Q.26 | ═C(R_{q})— | —N═ | ═C(R_{q})— | —N═ | ═C(R_{q})— |
| Q.27 | ═C(R_{q})— | —S— | —N═ | ═C(R_{q})— | — |
| Q.28 | ═C(R_{q})— | —O— | —N═ | ═C(R_{q})— | — |
| Q.29 | ═C(R_{q})— | —C(R_{q})═ | ═N— | —S— | — |
| Q.30 | ═C(R_{q})— | —C(R_{q})═ | ═N— | —O— | — |
| Q.31 | ═C(R_{q})— | —N(R_{q})— | —N═ | ═C(R_{q})— | — |

In the rings Q.1 to Q.31, R_{q} may be the same or different and is selected from the group: hydrogen or a radical R^{b}, in particular hydrogen, fluoro, chloro, bromo, methyl, methoxy, trifluoromethyl, trifluoromethoxy, methylthio, cyano, amino, phenyl, which is unsubstituted or may carry a halogen atom, and methylsulfonyl. Preferably, the rings Q.1 and Q.25 carry 0, 1, 2, or 3 substituents R_{q} other than hydrogen, preferably at the positions D, Z, and/or E. Also, preferably, the rings Q.2, Q.3, Q.4, Q.6, Q.8, Q.9, Q.16, Q.21, Q.23, Q.24 carry 0, 1 or 2 substituents R_{q} other than hydrogen, preferably at the positions: Z and E (in the case of the rings Q.2, Q.21), D and E (in the case of the rings Q.3, Q.23), or D and Z (in the case of the rings Q.4, Q.6, Q.8, Q.9, Q.16, Q.24). Also, preferably, the rings Q.5, Q.7, Q.10, Q.11, Q.12, Q.13, Q.14, Q.15, Q.17, Q.18, Q.19, Q.20, Q.22, Q.25, Q.29, Q.30 and Q.31 carry 0 or 1 substituents R_{q} other than hydrogen, preferably at the positions Z (in the case of the rings Q.5, Q.7, Q.10, Q.11, Q.12, Q.13, Q.14, Q.15, Q.17, Q.18, Q.19, Q.20, Q.26), D (in the case of the rings Q.29, Q.30, Q.31), or E (for Q.22). Q.27 and Q.28 preferably are substituted with hydrogen.

Amongst the compounds of this embodiment preference is given to those, wherein R⁶ is selected from a group Q*, wherein

Q* is 2-pyridyl, 3-pyridyl, 4-pyridyl, 2-thiophenyl (2-thienyl), 3-thiophenyl (3-thienyl), 2-furanyl (2-furyl), 3-furanyl (3-furyl), 3-isothiazolyl, 4-isothiazolyl, 5-isothiazolyl, 3-isoxazolyl, 4-isoxazolyl, 5-isoxazolyl, 2-oxazolyl, 4-oxazolyl, 5-oxazolyl, 2-thiazolyl, 4-thiazolyl, 5-thiazolyl.

A further particularly preferred embodiment of the present invention relates to compounds of the formula I, in particular to compounds of the formulae I-1, I-2, I-3 and I-4, more preferably compounds of the formulae I-1.a, I-1.b, I-1.c, I-1.d, I-2.a, I-2.b, I-2.c, I-2.d, I-3.a, I-3.b, I-3.c, I-3.d, I-4.a, I-4.b, I-4.c and I-4.d, wherein n and y are 0, R³ is hydrogen, A, R¹, R² and R⁴ are as defined above and R⁶ is phenyl as defined above, which is unsubstituted or substituted as given above and which carries in particular 1, 2 or 3 radicals R^{b} as defined above, with preference given to compounds, wherein phenyl carries one radical R^{b} in the 2-, 3- or 4-position or two identical or different radicals R^{b} in the 2,4-, 2,5-, 3,4-, 3,5- or 2,6-positions or three identical or different radicals R^{b} in the 2,3,4-, 2,3,5-, 2,4,5-, 3,4,5-, 2,3,6- or 2,4,6-positions.

With respect to their use, particular preference is given to the compounds I compiled in the tables below. Moreover, the groups mentioned for a substituent in the tables are on their own, independently of the combination in which they are mentioned, a particularly preferred embodiment of the substituent in question.

Table 1

Compounds of the formulae I-1 and I-2 wherein R¹ is hydrogen and the combination of R² and X—R⁶ in each case corresponds to a row of Table B.

Table 2

Compounds of the formulae I-1 and I-2 wherein R¹ is chloro and the combination of R² and X—R⁶ in each case corresponds to a row of Table B.

Table 3

Compounds of the formulae I-1 and I-2 wherein R¹ is bromo and the combination of R² and X—R⁶ in each case corresponds to a row of Table B.

Table 4

Compounds of the formulae I-1 and I-2 wherein R¹ is fluoro and the combination of R² and X—R⁶ in each case corresponds to a row of Table B.

Table 5

Compounds of the formulae I-1 and I-2 wherein R¹ is cyano and the combination of R² and X—R⁶ in each case corresponds to a row of Table B.

Table 6

Compounds of the formulae I-1 and I-2 wherein R¹ is methyl and the combination of R² and X—R⁶ in each case corresponds to a row of Table B.

Table 7

Compounds of the formulae I-1 and I-2 wherein R¹ is methylthio and the combination of R² and X—R⁶ in each case corresponds to a row of Table B.

Table 8

Compounds of the formulae I-1 and I-2 wherein R¹ is methoxy and the combination of R² and X—R⁶ in each case corresponds to a row of Table B.

Table 9

Compounds of the formulae I-1 and I-2 wherein R¹ is amino and the combination of R² and X—R⁶ in each case corresponds to a row of Table B.

Table 10

Compounds of the formulae I-1 and I-2 wherein R¹ is dimethylamino and the combination of R² and X—R⁶ in each case corresponds to a row of Table B.

Table 11

Compounds of the formulae I-1 and I-2 wherein R¹ is acetylamino and the combination of R² and X—R⁶ in each case corresponds to a row of Table B.

Table 12

Compounds of the formulae I-3 and I-4 wherein R¹ is hydrogen and the combination of R² and X—R⁶ in each case corresponds to a row of Table B.

Table 13

Compounds of the formulae I-3 and I-4 wherein R¹ is chloro and the combination of R² and X—R⁶ in each case corresponds to a row of Table B.

Table 14

Compounds of the formulae I-3 and I-4 wherein R¹ is bromo and the combination of R² and X—R⁶ in each case corresponds to a row of Table B.

Table 15

Compounds of the formulae I-3 and I-4 wherein R¹ is fluoro and the combination of R² and X—R⁶ in each case corresponds to a row of Table B.

Table 16

Compounds of the formulae I-3 and I-4 wherein R¹ is cyano and the combination of R² and X—R⁶ in each case corresponds to a row of Table B.

Table 17

Compounds of the formulae I-3 and I-4 wherein R¹ is methyl and the combination of R² and X—R⁶ in each case corresponds to a row of Table B.

Table 18

Compounds of the formulae I-3 and I-4 wherein R¹ is methylthio and the combination of R² and X—R⁶ in each case corresponds to a row of Table B.

Table 19

Compounds of the formulae I-3 and I-4 wherein R¹ is methoxy and the combination of R² and X—R⁶ in each case corresponds to a row of Table B.

Table 20

Compounds of the formulae I-3 and I-4 wherein R¹ is amino and the combination of R² and X—R⁶ in each case corresponds to a row of Table B.

Table 21

Compounds of the formulae I-3 and I-4 wherein R¹ is dimethylamino and the combination of R² and X—R⁶ in each case corresponds to a row of Table B.

Table 22

Compounds of the formulae I-3 and I-4 wherein R¹ is acetylamino and the combination of R² and X—R⁶ in each case corresponds to a row of Table B.

TABLE B

| No. | R² | X-R⁶ |
|---|---|---|
| I-1 | H | ethyl |
| I-2 | H | n-propyl |
| I-3 | H | 1-methyl-ethyl |
| I-4 | H | n-butyl |
| I-5 | H | 2-methyl-propyl |
| I-6 | H | 3-methyl-butyl |
| I-7 | H | 2,2-dimethyl-propyl |
| I-8 | H | 3,3-dimethyl-butyl |
| I-9 | H | 4,4-dimethyl-pentyl |
| I-10 | H | 2-ethyl-hexyl |
| I-11 | H | 3,5,5-trimethyl-hexyl |
| I-12 | H | 2-chloro-ethyl |
| I-13 | H | 2-tert-butoxy-propyl |
| I-14 | H | 2,3-diacetoxy-propyl |
| I-15 | H | 3-methoxycarbonyl- |
| | | propyl |
| I-16 | H | 3-methylsulfanyl- |
| | | propyl |
| I-17 | H | 2,3-dihydroxy- |
| | | propyl |
| I-18 | H | 2-hydroxy-butyl |
| I-19 | H | 2-acetoxy-butyl |
| I-20 | H | 2-benzyloxy-butyl |
| I-21 | H | 2-dimethylamino-2- |
| | | methyl-propyl |
| I-22 | H | 1-phenylethynyl- |
| | | butyl |
| I-23 | H | allyl |
| I-24 | H | 2,6-dimethyl-hept-5- |
| | | enyl |
| I-25 | H | (Z)-dec-4-enyl |
| I-26 | H | 2-(2,6,6-trimethyl- |
| | | cyclohex-1-enyl)- |
| | | ethyl |
| I-27 | H | 1-naphtalene- |
| | | methyl |
| I-28 | H | 2-naphtalene- |
| | | methyl |
| I-29 | H | phenethyl |
| I-30 | H | 2-phenyl-propyl |
| I-31 | H | 3-phenyl-propyl |
| I-32 | H | 4-benzo[1,3]dioxol- |
| | | 5-yl-butyl |
| I-33 | H | cyclopropyl-methyl |
| I-34 | H | 1-(4-chloro-phenyl)- |
| | | 1H-pyrrol-3-ylmethyl |
| I-35 | H | furan-2-ylmethyl |
| I-36 | H | furan-3-ylmethyl |
| I-37 | H | 3-dimethyl- |
| | | aminomethyl- |
| | | tetrahydro-furan-3- |
| | | ylmethyl |
| I-38 | H | 2,5-dihydro- |
| | | thiophen-3-ylmethyl |
| I-39 | H | 1-tert-butyl-1H- |
| | | pyrazol-4-ylmethyl |
| I-40 | H | 5-methyl-1H- |
| | | imidazol-4-ylmethyl |
| I-41 | H | 2-chloro-thiazol-5- |
| | | ylmethyl |
| I-42 | H | 3-isobutyl-isoxazol- |
| | | 5-ylmethyl |
| I-43 | H | 1-methyl-1H-pyrrol- |
| | | 2-ylmethyl |
| I-44 | H | tetrahydro-pyran-3- |
| | | ylmethyl |
| I-45 | H | tetrahydro-pyran-4- |
| | | ylmethyl |
| I-46 | H | tetrahydro-thio- |
| | | pyran-3-ylmethyl |
| I-47 | H | thiophen-2-ylmethyl |
| I-48 | H | pyridin-2-ylmethyl |
| I-49 | H | pyridin-3-ylmethyl |
| I-50 | H | pyridin-4-ylmethyl |
| I-51 | H | 6-chloro-pyridin-3- |
| | | ylmethyl |
| I-52 | H | 1H-indol-3-ylmethyl |
| I-53 | H | 3-chloro-1H-indol-2- |
| | | ylmethyl |
| I-54 | H | 2-(tetrahydro-pyran- |
| | | 4-yl)-ethyl |
| I-55 | H | 2,2-dimethyl-3- |
| | | pyridin-3-yl-propyl |
| I-56 | H | benzyl |
| I-57 | H | 2-amino-benzyl |
| I-58 | H | 2-bromo-benzyl |
| I-59 | H | 2-fluoro-benzyl |
| I-60 | H | 2-chloro-benzyl |
| I-61 | H | 2-cyano-benzyl |
| I-62 | H | 2-hydroxy-benzyl |
| I-63 | H | 2-methyl-benzyl |
| I-64 | H | 2-methoxy-benzyl |
| I-65 | H | 2-trifluoromethyl- |
| | | benzyl |
| I-66 | H | 2-trifluoromethoxy- |
| | | benzyl |
| I-67 | H | 2-diphenyl- |
| | | phosphino-benzyl |
| I-68 | H | 2-benzyloxy-benzyl |
| I-69 | H | 2,4-dichloro-benzyl |
| I-70 | H | 2,5-dichloro-benzyl |
| I-71 | H | 2,6-dichloro-benzyl |
| I-72 | H | 2-fluoro, 4-bromo- |
| | | benzyl |
| I-73 | H | 2-methyl,4-tert- |
| | | butyl-benzyl |
| I-74 | H | 2-chloro, |
| | | 6-methanesulfanyl- |
| | | benzyl |
| I-75 | H | 2-chloro, |
| | | 6-methanesulfinyl- |
| | | benzyl |
| I-76 | H | 2-methanesulfanyl, |
| | | 5-nitro-benzyl |
| I-77 | H | 2-methanesulfinyl, |
| | | 5-nitro-benzyl |
| I-78 | H | 3-amino-benzyl |
| I-79 | H | 3-bromo-benzyl |
| I-80 | H | 3-chloro-benzyl |
| I-81 | H | 3-fluoro-benzyl |
| I-82 | H | 3-cyano-benzyl |
| I-83 | H | 3-hydroxy-benzyl |
| I-84 | H | 3-methoxy-benzyl |
| I-85 | H | 3-methyl-benzyl |
| I-86 | H | 3-nitro-benzyl |
| I-87 | H | 3-benzyloxy-benzyl |
| I-88 | H | 3-trifluoromethyl- |
| | | benzyl |
| I-89 | H | 3-trifluoromethoxy- |
| | | benzyl |
| I-90 | H | 3,5-dichloro-benzyl |
| I-91 | H | 3,5-di-tert-butyl-4- |
| | | hydroxy-benzyl |
| I-92 | H | 3-bromo, 4-fluoro- |
| | | benzyl |
| I-93 | H | 3-bromo, |
| | | 6-fluoro-benzyl |
| I-94 | H | 3-fluoro, |
| | | 6-methanesulfinyl- |
| | | benzyl |
| I-95 | H | 3-fluoro, 6- |
| | | methanesulfanyl- |
| | | benzyl |
| I-96 | H | 3-chloro, |
| | | 6-methanesulfinyl- |
| | | benzyl |
| I-97 | H | 3-chloro, 6- |
| | | methanesulfanyl- |
| | | benzyl |
| I-98 | H | 4-amino-benzyl |
| I-99 | H | 4-benzyloxy-benzyl |
| I-100 | H | 4-bromo-benzyl |
| I-101 | H | 4-chloro-benzyl |
| I-102 | H | 4-fluoro-benzyl |
| I-103 | H | 4-cyano-benzyl |
| I-104 | H | 4-acetylamino- |
| | | benzyl |
| I-105 | H | 4-dimethylamino- |
| | | benzyl |
| I-106 | H | 4-hydroxy-benzyl |
| I-107 | H | 4-methyl-benzyl |
| I-108 | H | 4-methoxy-benzyl |
| I-109 | H | 4-methoxycarbonyl- |
| | | benzyl |
| I-110 | H | 4-methanesulfanyl- |
| | | benzyl |
| I-111 | H | 4-methanesulfinyl- |
| | | benzyl |
| I-112 | H | 4-N,N-dimethyl- |
| | | amino-benzyl |
| I-113 | H | 4-nitro-benzyl |
| I-114 | H | 4-tert-butyl-benzyl |
| I-115 | H | 4-trifluoromethyl- |
| | | benzyl |
| I-116 | H | 4-trifluoromethoxy- |
| | | benzyl |
| I-117 | H | 2,4,6-trimethoxy- |
| | | benzyl |
| I-118 | H | 2,3,4,5,6- |
| | | pentamethyl-benzyl |
| I-119 | H | 2-(4-fluoro-phenyl)- |
| | | 4-methyl-pentyl |
| I-120 | H | 2-[1-phenyl-meth- |
| | | (E)-ylidene]-heptyl |
| I-121 | H | 1-phenyl-prop-2- |
| | | ynyl |
| I-122 | H | 1-phenyl-3- |
| | | trimethylsilanyl- |
| | | prop-2-ynyl |
| I-123 | H | 4-ethoxycarbonyl-1- |
| | | phenyl-but-2-ynyl |
| I-124 | H | 4-methoxy-1-phenyl- |
| | | but-2-ynyl |
| I-125 | H | 4,4-dimethyl-1- |
| | | phenyl-pent-2-ynyl |
| I-126 | H | 1-phenyl-hex-2-ynyl |
| I-127 | H | 1-(4-chloro-phenyl)- |
| | | hex-2-ynyl |
| I-128 | H | 1-(4-methoxy- |
| | | phenyl)-hex-2-ynyl |
| I-129 | H | phenyl |
| I-130 | H | 2-chlorophenyl |
| I-131 | H | 2-fluorophenyl |
| I-132 | H | 2-trifluoromethyl- |
| | | phenyl |
| I-133 | H | 2-methylphenyl |
| I-134 | H | 2-methoxyphenyl |
| I-135 | H | 3-chlorophenyl |
| I-136 | H | 3-fluorophenyl |
| I-137 | H | 3-trifluoromethyl- |
| | | phenyl |
| I-138 | H | 3-methoxyphenyl |
| I-139 | H | 3-methylphenyl |
| I-140 | H | 4-chlorophenyl |
| I-141 | H | 4-fluorophenyl |
| I-142 | H | 4-methylphenyl |
| I-143 | H | 4-methoxyphenyl |
| I-144 | H | 4-trifluoromethyl- |
| | | phenyl |
| I-145 | H | 9H-fluoren-9-yl |
| I-146 | Cl | ethyl |
| I-147 | Cl | n-propyl |
| I-148 | Cl | 1-methyl-ethyl |
| I-149 | Cl | n-butyl |
| I-150 | Cl | 2-methyl-propyl |
| I-151 | Cl | 3-methyl-butyl |
| I-152 | Cl | 2,2-dimethyl-propyl |
| I-153 | Cl | 3,3-dimethyl-butyl |
| I-154 | Cl | 4,4-dimethyl-pentyl |
| I-155 | Cl | 2-ethyl-hexyl |
| I-156 | Cl | 3,5,5-trimethyl-hexyl |
| I-157 | Cl | 2-chloro-ethyl |
| I-158 | Cl | 2-tert-butoxy-propyl |
| I-159 | Cl | 2,3-diacetoxy-propyl |
| I-160 | Cl | 3-methoxycarbonyl- |
| | | propyl |
| I-161 | Cl | 3-methylsulfanyl- |
| | | propyl |
| I-162 | Cl | 2,3-dihydroxy- |
| | | propyl |
| I-163 | Cl | 2-hydroxy-butyl |
| I-164 | Cl | 2-acetoxy-butyl |
| I-165 | Cl | 2-benzyloxy-butyl |
| I-166 | Cl | 2-dimethylamino-2- |
| | | methyl-propyl |
| I-167 | Cl | 1-phenylethynyl- |
| | | butyl |
| I-168 | Cl | allyl |
| I-169 | Cl | 2,6-dimethyl-hept-5- |
| | | enyl |
| I-170 | Cl | (Z)-dec-4-enyl |
| I-171 | Cl | 2-(2,6,6-trimethyl- |
| | | cyclohex-1-enyl)- |
| | | ethyl |
| I-172 | Cl | 1-naphtalene- |
| | | methyl |
| I-173 | Cl | 2-naphtalene- |
| | | methyl |
| I-174 | Cl | phenethyl |
| I-175 | Cl | 2-phenyl-propyl |
| I-176 | Cl | 3-phenyl-propyl |
| I-177 | Cl | 4-benzo[1,3]dioxol- |
| | | 5-yl-butyl |
| I-178 | Cl | cyclopropyl-methyl |
| I-179 | Cl | 1-(4-chloro-phenyl)- |
| | | 1H-pyrrol-3-ylmethyl |
| I-180 | Cl | furan-2-ylmethyl |
| I-181 | Cl | furan-3-ylmethyl |
| I-182 | Cl | 3-dimethyl- |
| | | aminomethyl-tetra- |
| | | hydro-furan-3- |
| | | ylmethyl |
| I-183 | Cl | 2,5-dihydro- |
| | | thiophen-3-ylmethyl |
| I-184 | Cl | 1-tert-butyl-1H- |
| | | pyrazol-4-ylmethyl |
| I-185 | Cl | 5-methyl-1H- |
| | | imidazol-4-ylmethyl |
| I-186 | Cl | 2-chloro-thiazol-5- |
| | | ylmethyl |
| I-187 | Cl | 3-isobutyl-isoxazol- |
| | | 5-ylmethyl |
| I-188 | Cl | 1-methyl-1H-pyrrol- |
| | | 2-ylmethyl |
| I-189 | Cl | tetrahydro-pyran-3- |
| | | ylmethyl |
| I-190 | Cl | tetrahydro-pyran-4- |
| | | ylmethyl |
| I-191 | Cl | tetrahydro- |
| | | thiopyran-3-ylmethyl |
| I-192 | Cl | thiophen-2-ylmethyl |
| I-193 | Cl | pyridin-2-ylmethyl |
| I-194 | Cl | pyridin-3-ylmethyl |
| I-195 | Cl | pyridin-4-ylmethyl |
| I-196 | Cl | 6-chloro-pyridin-3- |
| | | ylmethyl |
| I-197 | Cl | 1H-indol-3-ylmethyl |
| I-198 | Cl | 3-chloro-1H-indol-2- |
| | | ylmethyl |
| I-199 | Cl | 2-(tetrahydro-pyran- |
| | | 4-yl)-ethyl |
| I-200 | Cl | 2,2-dimethyl-3- |
| | | pyridin-3-yl-propyl |
| I-201 | Cl | benzyl |
| I-202 | Cl | 2-amino-benzyl |
| I-203 | Cl | 2-bromo-benzyl |
| I-204 | Cl | 2-fluoro-benzyl |
| I-205 | Cl | 2-chloro-benzyl |
| I-206 | Cl | 2-cyano-benzyl |
| I-207 | Cl | 2-hydroxy-benzyl |
| I-208 | Cl | 2-methyl-benzyl |
| I-209 | Cl | 2-methoxy-benzyl |
| I-210 | Cl | 2-trifluoromethyl- |
| | | benzyl |
| I-211 | Cl | 2-trifluoromethoxy- |
| | | benzyl |
| I-212 | Cl | 2-diphenylphos- |
| | | phino-benzyl |
| I-213 | Cl | 2-benzyloxy-benzyl |
| I-214 | Cl | 2,4-dichloro-benzyl |
| I-215 | Cl | 2,5-dichloro-benzyl |
| I-216 | Cl | 2,6-dichloro-benzyl |
| I-217 | Cl | 2-fluoro, 4-bromo- |
| | | benzyl |
| I-218 | Cl | 2-methyl,4-tert- |
| | | butyl-benzyl |
| I-219 | Cl | 2-chloro, |
| | | 6-methanesulfanyl- |
| | | benzyl |
| I-220 | Cl | 2-chloro, |
| | | 6-methanesulfinyl- |
| | | benzyl |
| I-221 | Cl | 2-methanesulfanyl, |
| | | 5-nitro-benzyl |
| I-222 | Cl | 2-methanesulfinyl, |
| | | 5-nitro-benzyl |
| I-223 | Cl | 3-amino-benzyl |
| I-224 | Cl | 3-bromo-benzyl |
| I-225 | Cl | 3-chloro-benzyl |
| I-226 | Cl | 3-fluoro-benzyl |
| I-227 | Cl | 3-cyano-benzyl |
| I-228 | Cl | 3-hydroxy-benzyl |
| I-229 | Cl | 3-methoxy-benzyl |
| I-230 | Cl | 3-methyl-benzyl |
| I-231 | Cl | 3-nitro-benzyl |
| I-232 | Cl | 3-benzyloxy-benzyl |
| I-233 | Cl | 3-trifluoromethyl- |
| | | benzyl |
| I-234 | Cl | 3-trifluoromethoxy- |
| | | benzyl |
| I-235 | Cl | 3,5-dichloro-benzyl |
| I-236 | Cl | 3,5-di-tert-butyl-4- |
| | | hydroxy-benzyl |
| I-237 | Cl | 3-bromo, 4-fluoro- |
| | | benzyl |
| I-238 | Cl | 3-bromo, |
| | | 6-fluoro-benzyl |
| I-239 | Cl | 3-fluoro, |
| | | 6-methanesulfinyl- |
| | | benzyl |
| I-240 | Cl | 3-fluoro, 6- |
| | | methanesulfanyl- |
| | | benzyl |
| I-241 | Cl | 3-chloro, |
| | | 6-methanesulfinyl- |
| | | benzyl |
| I-242 | Cl | 3-chloro, 6- |
| | | methanesulfanyl- |
| | | benzyl |
| I-243 | Cl | 4-amino-benzyl |
| I-244 | Cl | 4-benzyloxy-benzyl |
| I-245 | Cl | 4-bromo-benzyl |
| I-246 | Cl | 4-chloro-benzyl |
| I-247 | Cl | 4-fluoro-benzyl |
| I-248 | Cl | 4-cyano-benzyl |
| I-249 | Cl | 4-acetylamino- |
| | | benzyl |
| I-250 | Cl | 4-dimethylamino- |
| | | benzyl |
| I-251 | Cl | 4-hydroxy-benzyl |
| I-252 | Cl | 4-methyl-benzyl |
| I-253 | Cl | 4-methoxy-benzyl |
| I-254 | Cl | 4-methoxycarbonyl- |
| | | benzyl |
| I-255 | Cl | 4-methanesulfanyl- |
| | | benzyl |
| I-256 | Cl | 4-methanesulfinyl- |
| | | benzyl |
| I-257 | Cl | 4-N,N-dimethyl- |
| | | amino-benzyl |
| I-258 | Cl | 4-nitro-benzyl |
| I-259 | Cl | 4-tert-butyl-benzyl |
| I-260 | Cl | 4-trifluoromethyl- |
| | | benzyl |
| I-261 | Cl | 4-trifluoromethoxy- |
| | | benzyl |
| I-262 | Cl | 2,4,6-trimethoxy- |
| | | benzyl |
| I-263 | Cl | 2,3,4,5,6-penta- |
| | | methyl-benzyl |
| I-264 | Cl | 2-(4-fluoro-phenyl)- |
| | | 4-methyl-pentyl |
| I-265 | Cl | 2-[1-phenyl-meth- |
| | | (E)-ylidene]-heptyl |
| I-266 | Cl | 1-phenyl-prop-2- |
| | | ynyl |
| I-267 | Cl | 1-phenyl-3- |
| | | trimethylsilanyl- |
| | | prop-2-ynyl |
| I-268 | Cl | 4-ethoxycarbonyl-1- |
| | | phenyl-but-2-ynyl |
| I-269 | Cl | 4-methoxy-1- |
| | | phenyl-but-2-ynyl |
| I-270 | Cl | 4,4-dimethyl-1- |
| | | phenyl-pent-2-ynyl |
| I-271 | Cl | 1-phenyl-hex-2-ynyl |
| I-272 | Cl | 1-(4-chloro-phenyl)- |
| | | hex-2-ynyl |
| I-273 | Cl | 1-(4-methoxy- |
| | | phenyl)-hex-2-ynyl |
| I-274 | Cl | phenyl |
| I-275 | Cl | 2-chlorophenyl |
| I-276 | Cl | 2-fluorophenyl |
| I-277 | Cl | 2-trifluoromethyl- |
| | | phenyl |
| I-278 | Cl | 2-methylphenyl |
| I-279 | Cl | 2-methoxyphenyl |
| I-280 | Cl | 3-chlorophenyl |
| I-281 | Cl | 3-fluorophenyl |
| I-282 | Cl | 3-trifluoromethyl- |
| | | phenyl |
| I-283 | Cl | 3-methoxyphenyl |
| I-284 | Cl | 3-methylphenyl |
| I-285 | Cl | 4-chlorophenyl |
| I-286 | Cl | 4-fluorophenyl |
| I-287 | Cl | 4-methylphenyl |
| I-288 | Cl | 4-methoxyphenyl |
| I-289 | Cl | 4-trifluoromethyl- |
| | | phenyl |
| I-290 | Cl | 9H-fluoren-9-yl |
| I-291 | F | ethyl |
| I-292 | F | n-propyl |
| I-293 | F | 1-methyl-ethyl |
| I-294 | F | n-butyl |
| I-295 | F | 2-methyl-propyl |
| I-296 | F | 3-methyl-butyl |
| I-297 | F | 2,2-dimethyl-propyl |
| I-298 | F | 3,3-dimethyl-butyl |
| I-299 | F | 4,4-dimethyl-pentyl |
| I-300 | F | 2-ethyl-hexyl |
| I-301 | F | 3,5,5-trimethyl-hexyl |
| I-302 | F | 2-chloro-ethyl |
| I-303 | F | 2-tert-butoxy-propyl |
| I-304 | F | 2,3-diacetoxy-propyl |
| I-305 | F | 3-methoxycarbonyl- |
| | | propyl |
| I-306 | F | 3-methylsulfanyl- |
| | | propyl |
| I-307 | F | 2,3-dihydroxy- |
| | | propyl |
| I-308 | F | 2-hydroxy-butyl |
| I-309 | F | 2-acetoxy-butyl |
| I-310 | F | 2-benzyloxy-butyl |
| I-311 | F | 2-dimethylamino-2- |
| | | methyl-propyl |
| I-312 | F | 1-phenylethynyl- |
| | | butyl |
| I-313 | F | allyl |
| I-314 | F | 2,6-dimethyl-hept-5- |
| | | enyl |
| I-315 | F | (Z)-dec-4-enyl |
| I-316 | F | 2-(2,6,6-trimethyl- |
| | | cyclohex-1-enyl)- |
| | | ethyl |
| I-317 | F | 1-naphtalene- |
| | | methyl |
| I-318 | F | 2-naphtalene- |
| | | methyl |
| I-319 | F | phenethyl |
| I-320 | F | 2-phenyl-propyl |
| I-321 | F | 3-phenyl-propyl |
| I-322 | F | 4-benzo[1,3]dioxol- |
| | | 5-yl-butyl |
| I-323 | F | cyclopropyl-methyl |
| I-324 | F | 1-(4-chloro-phenyl)- |
| | | 1H-pyrrol-3-ylmethyl |
| I-325 | F | furan-2-ylmethyl |
| I-326 | F | furan-3-ylmethyl |
| I-327 | F | 3-dimethyl- |
| | | aminomethyl- |
| | | tetrahydro-furan-3- |
| | | ylmethyl |
| I-328 | F | 2,5-dihydro- |
| | | thiophen-3-ylmethyl |
| I-329 | F | 1-tert-butyl-1H- |
| | | pyrazol-4-ylmethyl |
| I-330 | F | 5-methyl-1H- |
| | | imidazol-4-ylmethyl |
| I-331 | F | 2-chloro-thiazol-5- |
| | | ylmethyl |
| I-332 | F | 3-isobutyl-isoxazol- |
| | | 5-ylmethyl |
| I-333 | F | 1-methyl-1H-pyrrol- |
| | | 2-ylmethyl |
| I-334 | F | tetrahydro-pyran-3- |
| | | ylmethyl |
| I-335 | F | tetrahydro-pyran-4- |
| | | ylmethyl |
| I-336 | F | tetrahydro- |
| | | thiopyran-3-ylmethyl |
| I-337 | F | thiophen-2-ylmethyl |
| I-338 | F | pyridin-2-ylmethyl |
| I-339 | F | pyridin-3-ylmethyl |
| I-340 | F | pyridin-4-ylmethyl |
| I-341 | F | 6-chloro-pyridin-3- |
| | | ylmethyl |
| I-342 | F | 1H-indol-3-ylmethyl |
| I-343 | F | 3-chloro-1H-indol-2- |
| | | ylmethyl |
| I-344 | F | 2-(tetrahydro-pyran- |
| | | 4-yl)-ethyl |
| I-345 | F | 2,2-dimethyl-3- |
| | | pyridin-3-yl-propyl |
| I-346 | F | benzyl |
| I-347 | F | 2-amino-benzyl |
| I-348 | F | 2-bromo-benzyl |
| I-349 | F | 2-fluoro-benzyl |
| I-350 | F | 2-chloro-benzyl |
| I-351 | F | 2-cyano-benzyl |
| I-352 | F | 2-hydroxy-benzyl |
| I-353 | F | 2-methyl-benzyl |
| I-354 | F | 2-methoxy-benzyl |
| I-355 | F | 2-trifluoromethyl- |
| | | benzyl |
| I-356 | F | 2-trifluoromethoxy- |
| | | benzyl |
| I-357 | F | 2-diphenylphosphino- |
| | | benzyl |
| I-358 | F | 2-benzyloxy-benzyl |
| I-359 | F | 2,4-dichloro-benzyl |
| I-360 | F | 2,5-dichloro-benzyl |
| I-361 | F | 2,6-dichloro-benzyl |
| I-362 | F | 2-fluoro, 4-bromo- |
| | | benzyl |
| I-363 | F | 2-methyl,4-tert- |
| | | butyl-benzyl |
| I-364 | F | 2-chloro, |
| | | 6-methanesulfanyl- |
| | | benzyl |
| I-365 | F | 2-chloro, |
| | | 6-methanesulfinyl- |
| | | benzyl |
| I-366 | F | 2-methanesulfanyl, |
| | | 5-nitro-benzyl |
| I-367 | F | 2-methanesulfinyl, |
| | | 5-nitro-benzyl |
| I-368 | F | 3-amino-benzyl |
| I-369 | F | 3-bromo-benzyl |
| I-370 | F | 3-chloro-benzyl |
| I-371 | F | 3-fluoro-benzyl |
| I-372 | F | 3-cyano-benzyl |
| I-373 | F | 3-hydroxy-benzyl |
| I-374 | F | 3-methoxy-benzyl |
| I-375 | F | 3-methyl-benzyl |
| I-376 | F | 3-nitro-benzyl |
| I-377 | F | 3-benzyloxy-benzyl |
| I-378 | F | 3-trifluoromethyl- |
| | | benzyl |
| I-379 | F | 3-trifluoromethoxy- |
| | | benzyl |
| I-380 | F | 3,5-dichloro-benzyl |
| I-381 | F | 3,5-di-tert-butyl-4- |
| | | hydroxy-benzyl |
| I-382 | F | 3-bromo, 4-fluoro- |
| | | benzyl |
| I-383 | F | 3-bromo, |
| | | 6-fluoro-benzyl |
| I-384 | F | 3-fluoro, |
| | | 6-methanesulfinyl- |
| | | benzyl |
| I-385 | F | 3-fluoro, 6- |
| | | methanesulfanyl- |
| | | benzyl |
| I-386 | F | 3-chloro, |
| | | 6-methanesulfinyl- |
| | | benzyl |
| I-387 | F | 3-chloro, 6- |
| | | methanesulfanyl- |
| | | benzyl |
| I-388 | F | 4-amino-benzyl |
| I-389 | F | 4-benzyloxy-benzyl |
| I-390 | F | 4-bromo-benzyl |
| I-391 | F | 4-chloro-benzyl |
| I-392 | F | 4-fluoro-benzyl |
| I-393 | F | 4-cyano-benzyl |
| I-394 | F | 4-acetylamino- |
| | | benzyl |
| I-395 | F | 4-dimethylamino- |
| | | benzyl |
| I-396 | F | 4-hydroxy-benzyl |
| I-397 | F | 4-methyl-benzyl |
| I-398 | F | 4-methoxy-benzyl |
| I-399 | F | 4-methoxycarbonyl- |
| | | benzyl |
| I-400 | F | 4-methanesulfanyl- |
| | | benzyl |
| I-401 | F | 4-methanesulfinyl- |
| | | benzyl |
| I-402 | F | 4-N,N-dimethyl- |
| | | amino-benzyl |
| I-403 | F | 4-nitro-benzyl |
| I-404 | F | 4-tert-butyl-benzyl |
| I-405 | F | 4-trifluoromethyl- |
| | | benzyl |
| I-406 | F | 4-trifluoromethoxy- |
| | | benzyl |
| I-407 | F | 2,4,6-trimethoxy- |
| | | benzyl |
| I-408 | F | 2,3,4,5,6- |
| | | pentamethyl-benzyl |
| I-409 | F | 2-(4-fluoro-phenyl)- |
| | | 4-methyl-pentyl |
| I-410 | F | 2-[1-phenyl-meth- |
| | | (E)-ylidene]-heptyl |
| I-411 | F | 1-phenyl-prop-2- |
| | | ynyl |
| I-412 | F | 1-phenyl-3- |
| | | trimethylsilanyl- |
| | | prop-2-ynyl |
| I-413 | F | 4-ethoxycarbonyl-1- |
| | | phenyl-but-2-ynyl |
| I-414 | F | 4-methoxy-1- |
| | | phenyl-but-2-ynyl |
| I-415 | F | 4,4-dimethyl-1- |
| | | phenyl-pent-2-ynyl |
| I-416 | F | 1-phenyl-hex-2-ynyl |
| I-417 | F | 1-(4-chloro-phenyl)- |
| | | hex-2-ynyl |
| I-418 | F | 1-(4-methoxy- |
| | | phenyl)-hex-2-ynyl |
| I-419 | F | phenyl |
| I-420 | F | 2-chlorophenyl |
| I-421 | F | 2-fluorophenyl |
| I-422 | F | 2-trifluoromethyl- |
| | | phenyl |
| I-423 | F | 2-methylphenyl |
| I-424 | F | 2-methoxyphenyl |
| I-425 | F | 3-chlorophenyl |
| I-426 | F | 3-fluorophenyl |
| I-427 | F | 3-trifluoromethyl- |
| | | phenyl |
| I-428 | F | 3-methoxyphenyl |
| I-429 | F | 3-methylphenyl |
| I-430 | F | 4-chlorophenyl |
| I-431 | F | 4-fluorophenyl |
| I-432 | F | 4-methylphenyl |
| I-433 | F | 4-methoxyphenyl |
| I-434 | F | 4-trifluoromethyl- |
| | | phenyl |
| I-435 | F | 9H-fluoren-9-yl |
| I-436 | Br | ethyl |
| I-437 | Br | n-propyl |
| I-438 | Br | 1-methyl-ethyl |
| I-439 | Br | n-butyl |
| I-440 | Br | 2-methyl-propyl |
| I-441 | Br | 3-methyl-butyl |
| I-442 | Br | 2,2-dimethyl-propyl |
| I-443 | Br | 3,3-dimethyl-butyl |
| I-444 | Br | 4,4-dimethyl-pentyl |
| I-445 | Br | 2-ethyl-hexyl |
| I-446 | Br | 3,5,5-trimethyl-hexyl |
| I-447 | Br | 2-chloro-ethyl |
| I-448 | Br | 2-tert-butoxy-propyl |
| I-449 | Br | 2,3-diacetoxy-propyl |
| I-450 | Br | 3-methoxycarbonyl- |
| | | propyl |
| I-451 | Br | 3-methylsulfanyl- |
| | | propyl |
| I-452 | Br | 2,3-dihydroxy- |
| | | propyl |
| I-453 | Br | 2-hydroxy-butyl |
| I-454 | Br | 2-acetoxy-butyl |
| I-455 | Br | 2-benzyloxy-butyl |
| I-456 | Br | 2-dimethylamino-2- |
| | | methyl-propyl |
| I-457 | Br | 1-phenylethynyl- |
| | | butyl |
| I-458 | Br | allyl |
| I-459 | Br | 2,6-dimethyl-hept-5- |
| | | enyl |
| I-460 | Br | (Z)-dec-4-enyl |
| I-461 | Br | 2-(2,6,6-trimethyl- |
| | | cyclohex-1-enyl)- |
| | | ethyl |
| I-462 | Br | 1-naphtalene- |
| | | methyl |
| I-463 | Br | 2-naphtalene- |
| | | methyl |
| I-464 | Br | phenethyl |
| I-465 | Br | 2-phenyl-propyl |
| I-466 | Br | 3-phenyl-propyl |
| I-467 | Br | 4-benzo[1,3]dioxol- |
| | | 5-yl-butyl |
| I-468 | Br | cyclopropyl-methyl |
| I-469 | Br | 1-(4-chloro-phenyl)- |
| | | 1H-pyrrol-3-ylmethyl |
| I-470 | Br | furan-2-ylmethyl |
| I-471 | Br | furan-3-ylmethyl |
| I-472 | Br | 3-dimethyl- |
| | | aminomethyl- |
| | | tetrahydro-furan-3- |
| | | ylmethyl |
| I-473 | Br | 2,5-dihydro- |
| | | thiophen-3-ylmethyl |
| I-474 | Br | 1-tert-butyl-1H- |
| | | pyrazol-4-ylmethyl |
| I-475 | Br | 5-methyl-1H- |
| | | imidazol-4-ylmethyl |
| I-476 | Br | 2-chloro-thiazol-5- |
| | | ylmethyl |
| I-477 | Br | 3-isobutyl-isoxazol- |
| | | 5-ylmethyl |
| I-478 | Br | 1-methyl-1H-pyrrol- |
| | | 2-ylmethyl |
| I-479 | Br | tetrahydro-pyran-3- |
| | | ylmethyl |
| I-480 | Br | tetrahydro-pyran-4- |
| | | ylmethyl |
| I-481 | Br | tetrahydro- |
| | | thiopyran-3-ylmethyl |
| I-482 | Br | thiophen-2-ylmethyl |
| I-483 | Br | pyridin-2-ylmethyl |
| I-484 | Br | pyridin-3-ylmethyl |
| I-485 | Br | pyridin-4-ylmethyl |
| I-486 | Br | 6-chloro-pyridin-3- |
| | | ylmethyl |
| I-487 | Br | 1H-indol-3-ylmethyl |
| I-488 | Br | 3-chloro-1H-indol-2- |
| | | ylmethyl |
| I-489 | Br | 2-(tetrahydro-pyran- |
| | | 4-yl)-ethyl |
| I-490 | Br | 2,2-dimethyl-3- |
| | | pyridin-3-yl-propyl |
| I-491 | Br | benzyl |
| I-492 | Br | 2-amino-benzyl |
| I-493 | Br | 2-bromo-benzyl |
| I-494 | Br | 2-fluoro-benzyl |
| I-495 | Br | 2-chloro-benzyl |
| I-496 | Br | 2-cyano-benzyl |
| I-497 | Br | 2-hydroxy-benzyl |
| I-498 | Br | 2-methyl-benzyl |
| I-499 | Br | 2-methoxy-benzyl |
| I-500 | Br | 2-trifluoromethyl- |
| | | benzyl |
| I-501 | Br | 2-trifluoromethoxy- |
| | | benzyl |
| I-502 | Br | 2-diphenyl- |
| | | phosphino-benzyl |
| I-503 | Br | 2-benzyloxy-benzyl |
| I-504 | Br | 2,4-dichloro-benzyl |
| I-505 | Br | 2,5-dichloro-benzyl |
| I-506 | Br | 2,6-dichloro-benzyl |
| I-507 | Br | 2-fluoro, 4-bromo- |
| | | benzyl |
| I-508 | Br | 2-methyl,4-tert- |
| | | butyl-benzyl |
| I-509 | Br | 2-chloro, |
| | | 6-methanesulfanyl- |
| | | benzyl |
| I-510 | Br | 2-chloro, |
| | | 6-methanesulfinyl- |
| | | benzyl |
| I-511 | Br | 2-methanesulfanyl, |
| | | 5-nitro-benzyl |
| I-512 | Br | 2-methanesulfinyl, |
| | | 5-nitro-benzyl |
| I-513 | Br | 3-amino-benzyl |
| I-514 | Br | 3-bromo-benzyl |
| I-515 | Br | 3-chloro-benzyl |
| I-516 | Br | 3-fluoro-benzyl |
| I-517 | Br | 3-cyano-benzyl |
| I-518 | Br | 3-hydroxy-benzyl |
| I-519 | Br | 3-methoxy-benzyl |
| I-520 | Br | 3-methyl-benzyl |
| I-521 | Br | 3-nitro-benzyl |
| I-522 | Br | 3-benzyloxy-benzyl |
| I-523 | Br | 3-trifluoromethyl- |
| | | benzyl |
| I-524 | Br | 3-trifluoromethoxy- |
| | | benzyl |
| I-525 | Br | 3,5-dichloro-benzyl |
| I-526 | Br | 3,5-di-tert-butyl-4- |
| | | hydroxy-benzyl |
| I-527 | Br | 3-bromo, 4-fluoro- |
| | | benzyl |
| I-528 | Br | 3-bromo, |
| | | 6-fluoro-benzyl |
| I-529 | Br | 3-fluoro, |
| | | 6-methanesulfinyl- |
| | | benzyl |
| I-530 | Br | 3-fluoro, 6- |
| | | methanesulfanyl- |
| | | benzyl |
| I-531 | Br | 3-chloro, |
| | | 6-methanesulfinyl- |
| | | benzyl |
| I-532 | Br | 3-chloro, 6- |
| | | methanesulfanyl- |
| | | benzyl |
| I-533 | Br | 4-amino-benzyl |
| I-534 | Br | 4-benzyloxy-benzyl |
| I-535 | Br | 4-bromo-benzyl |
| I-536 | Br | 4-chloro-benzyl |
| I-537 | Br | 4-fluoro-benzyl |
| I-538 | Br | 4-cyano-benzyl |
| I-539 | Br | 4-acetylamino- |
| | | benzyl |
| I-540 | Br | 4-dimethylamino- |
| | | benzyl |
| I-541 | Br | 4-hydroxy-benzyl |
| I-542 | Br | 4-methyl-benzyl |
| I-543 | Br | 4-methoxy-benzyl |
| I-544 | Br | 4-methoxycarbonyl- |
| | | benzyl |
| I-545 | Br | 4-methanesulfanyl- |
| | | benzyl |
| I-546 | Br | 4-methanesulfinyl- |
| | | benzyl |
| I-547 | Br | 4-N,N-dimethyl- |
| | | amino-benzyl |
| I-548 | Br | 4-nitro-benzyl |
| I-549 | Br | 4-tert-butyl-benzyl |
| I-550 | Br | 4-trifluoromethyl- |
| | | benzyl |
| I-551 | Br | 4-trifluoromethoxy- |
| | | benzyl |
| I-552 | Br | 2,4,6-trimethoxy- |
| | | benzyl |
| I-553 | Br | 2,3,4,5,6- |
| | | pentamethyl-benzyl |
| I-554 | Br | 2-(4-fluoro-phenyl)- |
| | | 4-methyl-pentyl |
| I-555 | Br | 2-[1-phenyl-meth- |
| | | (E)-ylidene]-heptyl |
| I-556 | Br | 1-phenyl-prop-2- |
| | | ynyl |
| I-557 | Br | 1-phenyl-3- |
| | | trimethylsilanyl- |
| | | prop-2-ynyl |
| I-558 | Br | 4-ethoxycarbonyl-1- |
| | | phenyl-but-2-ynyl |
| I-559 | Br | 4-methoxy-1- |
| | | phenyl-but-2-ynyl |
| I-560 | Br | 4,4-dimethyl-1- |
| | | phenyl-pent-2-ynyl |
| I-561 | Br | 1-phenyl-hex-2-ynyl |
| I-562 | Br | 1-(4-chloro-phenyl)- |
| | | hex-2-ynyl |
| I-563 | Br | 1-(4-methoxy- |
| | | phenyl)-hex-2-ynyl |
| I-564 | Br | phenyl |
| I-565 | Br | 2-chlorophenyl |
| I-566 | Br | 2-fluorophenyl |
| I-567 | Br | 2-trifluoromethyl- |
| | | phenyl |
| I-568 | Br | 2-methylphenyl |
| I-569 | Br | 2-methoxyphenyl |
| I-570 | Br | 3-chlorophenyl |
| I-571 | Br | 3-fluorophenyl |
| I-572 | Br | 3-trifluoromethyl- |
| | | phenyl |
| I-573 | Br | 3-methoxyphenyl |
| I-574 | Br | 3-methylphenyl |
| I-575 | Br | 4-chlorophenyl |
| I-576 | Br | 4-fluorophenyl |
| I-577 | Br | 4-methylphenyl |
| I-578 | Br | 4-methoxyphenyl |
| I-579 | Br | 4-trifluoromethyl- |
| | | phenyl |
| I-580 | Br | 9H-fluoren-9-yl |
| I-581 | CN | ethyl |
| I-582 | CN | n-propyl |
| I-583 | CN | 1-methyl-ethyl |
| I-584 | CN | n-butyl |
| I-585 | CN | 2-methyl-propyl |
| I-586 | CN | 3-methyl-butyl |
| I-587 | CN | 2,2-dimethyl-propyl |
| I-588 | CN | 3,3-dimethyl-butyl |
| I-589 | CN | 4,4-dimethyl-pentyl |
| I-590 | CN | 2-ethyl-hexyl |
| I-591 | CN | 3,5,5-trimethyl-hexyl |
| I-592 | CN | 2-chloro-ethyl |
| I-593 | CN | 2-tert-butoxy-propyl |
| I-594 | CN | 2,3-diacetoxy-propyl |
| I-595 | CN | 3-methoxycarbonyl- |
| | | propyl |
| I-596 | CN | 3-methylsulfanyl- |
| | | propyl |
| I-597 | CN | 2,3-dihydroxy- |
| | | propyl |
| I-598 | CN | 2-hydroxy-butyl |
| I-599 | CN | 2-acetoxy-butyl |
| I-600 | CN | 2-benzyloxy-butyl |
| I-601 | CN | 2-dimethylamino-2- |
| | | methyl-propyl |
| I-602 | CN | 1-phenylethynyl- |
| | | butyl |
| I-603 | CN | allyl |
| I-604 | CN | 2,6-dimethyl-hept-5- |
| | | enyl |
| I-605 | CN | (Z)-dec-4-enyl |
| I-606 | CN | 2-(2,6,6-trimethyl- |
| | | cyclohex-1-enyl)- |
| | | ethyl |
| I-607 | CN | 1-naphtalene- |
| | | methyl |
| I-608 | CN | 2-naphtalene- |
| | | methyl |
| I-609 | CN | phenethyl |
| I-610 | CN | 2-phenyl-propyl |
| I-611 | CN | 3-phenyl-propyl |
| I-612 | CN | 4-benzo[1,3]dioxol- |
| | | 5-yl-butyl |
| I-613 | CN | cyclopropyl-methyl |
| I-614 | CN | 1-(4-chloro-phenyl)- |
| | | 1H-pyrrol-3-ylmethyl |
| I-615 | CN | furan-2-ylmethyl |
| I-616 | CN | furan-3-ylmethyl |
| I-617 | CN | 3-dimethyl- |
| | | aminomethyl- |
| | | tetrahydro-furan-3- |
| | | ylmethyl |
| I-618 | CN | 2,5-dihydro- |
| | | thiophen-3-ylmethyl |
| I-619 | CN | 1-tert-butyl-1H- |
| | | pyrazol-4-ylmethyl |
| I-620 | CN | 5-methyl-1H- |
| | | imidazol-4-ylmethyl |
| I-621 | CN | 2-chloro-thiazol-5- |
| | | ylmethyl |
| I-622 | CN | 3-isobutyl-isoxazol- |
| | | 5-ylmethyl |
| I-623 | CN | 1-methyl-1H-pyrrol- |
| | | 2-ylmethyl |
| I-624 | CN | tetrahydro-pyran-3- |
| | | ylmethyl |
| I-625 | CN | tetrahydro-pyran-4- |
| | | ylmethyl |
| I-626 | CN | tetrahydro- |
| | | thiopyran-3-ylmethyl |
| I-627 | CN | thiophen-2-ylmethyl |
| I-628 | CN | pyridin-2-ylmethyl |
| I-629 | CN | pyridin-3-ylmethyl |
| I-630 | CN | pyridin-4-ylmethyl |
| I-631 | CN | 6-chloro-pyridin-3- |
| | | ylmethyl |
| I-632 | CN | 1H-indol-3-ylmethyl |
| I-633 | CN | 3-chloro-1H-indol-2- |
| | | ylmethyl |
| I-634 | CN | 2-(tetrahydro-pyran- |
| | | 4-yl)-ethyl |
| I-635 | CN | 2,2-dimethyl-3- |
| | | pyridin-3-yl-propyl |
| I-636 | CN | benzyl |
| I-637 | CN | 2-amino-benzyl |
| I-638 | CN | 2-bromo-benzyl |
| I-639 | CN | 2-fluoro-benzyl |
| I-640 | CN | 2-chloro-benzyl |
| I-641 | CN | 2-cyano-benzyl |
| I-642 | CN | 2-hydroxy-benzyl |
| I-643 | CN | 2-methyl-benzyl |
| I-644 | CN | 2-methoxy-benzyl |
| I-645 | CN | 2-trifluoromethyl- |
| | | benzyl |
| I-646 | CN | 2-trifluoromethoxy- |
| | | benzyl |
| I-647 | CN | 2-diphenyl- |
| | | phosphino-benzyl |
| I-648 | CN | 2-benzyloxy-benzyl |
| I-649 | CN | 2,4-dichloro-benzyl |
| I-650 | CN | 2,5-dichloro-benzyl |
| I-651 | CN | 2,6-dichloro-benzyl |
| I-652 | CN | 2-fluoro, 4-bromo- |
| | | benzyl |
| I-653 | CN | 2-methyl,4-tert- |
| | | butyl-benzyl |
| I-654 | CN | 2-chloro, |
| | | 6-methanesulfanyl- |
| | | benzyl |
| I-655 | CN | 2-chloro, |
| | | 6-methanesulfinyl- |
| | | benzyl |
| I-656 | CN | 2-methanesulfanyl, |
| | | 5-nitro-benzyl |
| I-657 | CN | 2-methanesulfinyl, |
| | | 5-nitro-benzyl |
| I-658 | CN | 3-amino-benzyl |
| I-659 | CN | 3-bromo-benzyl |
| I-660 | CN | 3-chloro-benzyl |
| I-661 | CN | 3-fluoro-benzyl |
| I-662 | CN | 3-cyano-benzyl |
| I-663 | CN | 3-hydroxy-benzyl |
| I-664 | CN | 3-methoxy-benzyl |
| I-665 | CN | 3-methyl-benzyl |
| I-666 | CN | 3-nitro-benzyl |
| I-667 | CN | 3-benzyloxy-benzyl |
| I-668 | CN | 3-trifluoromethyl- |
| | | benzyl |
| I-669 | CN | 3-trifluoromethoxy- |
| | | benzyl |
| I-670 | CN | 3,5-dichloro-benzyl |
| I-671 | CN | 3,5-di-tert-butyl-4- |
| | | hydroxy-benzyl |
| I-672 | CN | 3-bromo, 4-fluoro- |
| | | benzyl |
| I-673 | CN | 3-bromo, |
| | | 6-fluoro-benzyl |
| I-674 | CN | 3-fluoro, |
| | | 6-methanesulfinyl- |
| | | benzyl |
| I-675 | CN | 3-fluoro, 6- |
| | | methanesulfanyl- |
| | | benzyl |
| I-676 | CN | 3-chloro, |
| | | 6-methanesulfinyl- |
| | | benzyl |
| I-677 | CN | 3-chloro, 6- |
| | | methanesulfanyl- |
| | | benzyl |
| I-678 | CN | 4-amino-benzyl |
| I-679 | CN | 4-benzyloxy-benzyl |
| I-680 | CN | 4-bromo-benzyl |
| I-681 | CN | 4-chloro-benzyl |
| I-682 | CN | 4-fluoro-benzyl |
| I-683 | CN | 4-cyano-benzyl |
| I-684 | CN | 4-acetylamino- |
| | | benzyl |
| I-685 | CN | 4-dimethylamino- |
| | | benzyl |
| I-686 | CN | 4-hydroxy-benzyl |
| I-687 | CN | 4-methyl-benzyl |
| I-688 | CN | 4-methoxy-benzyl |
| I-689 | CN | 4-methoxycarbonyl- |
| | | benzyl |
| I-690 | CN | 4-methanesulfanyl- |
| | | benzyl |
| I-691 | CN | 4-methanesulfinyl- |
| | | benzyl |
| I-692 | CN | 4-N,N-dimethyl- |
| | | amino-benzyl |
| I-693 | CN | 4-nitro-benzyl |
| I-694 | CN | 4-tert-butyl-benzyl |
| I-695 | CN | 4-trifluoromethyl- |
| | | benzyl |
| I-696 | CN | 4-trifluoromethoxy- |
| | | benzyl |
| I-697 | CN | 2,4,6-trimethoxy- |
| | | benzyl |
| I-698 | CN | 2,3,4,5,6- |
| | | pentamethyl-benzyl |
| I-699 | CN | 2-(4-fluoro-phenyl)- |
| | | 4-methyl-pentyl |
| I-700 | CN | 2-[1-phenyl-meth- |
| | | (E)-ylidene]-heptyl |
| I-701 | CN | 1-phenyl-prop-2- |
| | | ynyl |
| I-702 | CN | 1-phenyl-3- |
| | | trimethylsilanyl- |
| | | prop-2-ynyl |
| I-703 | CN | 4-ethoxycarbonyl-1- |
| | | phenyl-but-2-ynyl |
| I-704 | CN | 4-methoxy-1- |
| | | phenyl-but-2-ynyl |
| I-705 | CN | 4,4-dimethyl-1- |
| | | phenyl-pent-2-ynyl |
| I-706 | CN | 1-phenyl-hex-2-ynyl |
| I-707 | CN | 1-(4-chloro-phenyl)- |
| | | hex-2-ynyl |
| I-708 | CN | 1-(4-methoxy- |
| | | phenyl)-hex-2-ynyl |
| I-709 | CN | phenyl |
| I-710 | CN | 2-chlorophenyl |
| I-711 | CN | 2-fluorophenyl |
| I-712 | CN | 2-trifluoromethyl- |
| | | phenyl |
| I-713 | CN | 2-methylphenyl |
| I-714 | CN | 2-methoxyphenyl |
| I-715 | CN | 3-chlorophenyl |
| I-716 | CN | 3-fluorophenyl |
| I-717 | CN | 3-trifluoromethyl- |
| | | phenyl |
| I-718 | CN | 3-methoxyphenyl |
| I-719 | CN | 3-methylphenyl |
| I-720 | CN | 4-chlorophenyl |
| I-721 | CN | 4-fluorophenyl |
| I-722 | CN | 4-methylphenyl |
| I-723 | CN | 4-methoxyphenyl |
| I-724 | CN | 4-trifluoromethyl- |
| | | phenyl |
| I-725 | CN | 9H-fluoren-9-yl |
| I-726 | NH₂ | ethyl |
| I-727 | NH₂ | n-propyl |
| I-728 | NH₂ | 1-methyl-ethyl |
| I-729 | NH₂ | n-butyl |
| I-730 | NH₂ | 2-methyl-propyl |
| I-731 | NH₂ | 3-methyl-butyl |
| I-732 | NH₂ | 2,2-dimethyl-propyl |
| I-733 | NH₂ | 3,3-dimethyl-butyl |
| I-734 | NH₂ | 4,4-dimethyl-pentyl |
| I-735 | NH₂ | 2-ethyl-hexyl |
| I-736 | NH₂ | 3,5,5-trimethyl-hexyl |
| I-737 | NH₂ | 2-chloro-ethyl |
| I-738 | NH₂ | 2-tert-butoxy-propyl |
| I-739 | NH₂ | 2,3-diacetoxy-propyl |
| I-740 | NH₂ | 3-methoxycarbonyl- |
| | | propyl |
| I-741 | NH₂ | 3-methylsulfanyl- |
| | | propyl |
| I-742 | NH₂ | 2,3-dihydroxy- |
| | | propyl |
| I-743 | NH₂ | 2-hydroxy-butyl |
| I-744 | NH₂ | 2-acetoxy-butyl |
| I-745 | NH₂ | 2-benzyloxy-butyl |
| I-746 | NH₂ | 2-dimethylamino-2- |
| | | methyl-propyl |
| I-747 | NH₂ | 1-phenylethynyl- |
| | | butyl |
| I-748 | NH₂ | allyl |
| I-749 | NH₂ | 2,6-dimethyl-hept-5- |
| | | enyl |
| I-750 | NH₂ | (Z)-dec-4-enyl |
| I-751 | NH₂ | 2-(2,6,6-trimethyl- |
| | | cyclohex-1-enyl)- |
| | | ethyl |
| I-752 | NH₂ | 1-naphtalene- |
| | | methyl |
| I-753 | NH₂ | 2-naphtalene- |
| | | methyl |
| I-754 | NH₂ | phenethyl |
| I-755 | NH₂ | 2-phenyl-propyl |
| I-756 | NH₂ | 3-phenyl-propyl |
| I-757 | NH₂ | 4-benzo[1,3]dioxol- |
| | | 5-yl-butyl |
| I-758 | NH₂ | cyclopropyl-methyl |
| I-759 | NH₂ | 1-(4-chloro-phenyl)- |
| | | 1H-pyrrol-3-ylmethyl |
| I-760 | NH₂ | furan-2-ylmethyl |
| I-761 | NH₂ | furan-3-ylmethyl |
| I-762 | NH₂ | 3-dimethyl- |
| | | aminomethyl- |
| | | tetrahydro-furan-3- |
| | | ylmethyl |
| I-763 | NH₂ | 2,5-dihydro- |
| | | thiophen-3-ylmethyl |
| I-764 | NH₂ | 1-tert-butyl-1H- |
| | | pyrazol-4-ylmethyl |
| I-765 | NH₂ | 5-methyl-1H- |
| | | imidazol-4-ylmethyl |
| I-766 | NH₂ | 2-chloro-thiazol-5- |
| | | ylmethyl |
| I-767 | NH₂ | 3-isobutyl-isoxazol- |
| | | 5-ylmethyl |
| I-768 | NH₂ | 1-methyl-1H-pyrrol- |
| | | 2-ylmethyl |
| I-769 | NH₂ | tetrahydro-pyran-3- |
| | | ylmethyl |
| I-770 | NH₂ | tetrahydro-pyran-4- |
| | | ylmethyl |
| I-771 | NH₂ | tetrahydro- |
| | | thiopyran-3-ylmethyl |
| I-772 | NH₂ | thiophen-2-ylmethyl |
| I-773 | NH₂ | pyridin-2-ylmethyl |
| I-774 | NH₂ | pyridin-3-ylmethyl |
| I-775 | NH₂ | pyridin-4-ylmethyl |
| I-776 | NH₂ | 6-chloro-pyridin-3- |
| | | ylmethyl |
| I-777 | NH₂ | 1H-indol-3-ylmethyl |
| I-778 | NH₂ | 3-chloro-1H-indol-2- |
| | | ylmethyl |
| I-779 | NH₂ | 2-(tetrahydro-pyran- |
| | | 4-yl)-ethyl |
| I-780 | NH₂ | 2,2-dimethyl-3- |
| | | pyridin-3-yl-propyl |
| I-781 | NH₂ | benzyl |
| I-782 | NH₂ | 2-amino-benzyl |
| I-783 | NH₂ | 2-bromo-benzyl |
| I-784 | NH₂ | 2-fluoro-benzyl |
| I-785 | NH₂ | 2-chloro-benzyl |
| I-786 | NH₂ | 2-cyano-benzyl |
| I-787 | NH₂ | 2-hydroxy-benzyl |
| I-788 | NH₂ | 2-methyl-benzyl |
| I-789 | NH₂ | 2-methoxy-benzyl |
| I-790 | NH₂ | 2-trifluoromethyl- |
| | | benzyl |
| I-791 | NH₂ | 2-trifluoromethoxy- |
| | | benzyl |
| I-792 | NH₂ | 2-diphenyl- |
| | | phosphino-benzyl |
| I-793 | NH₂ | 2-benzyloxy-benzyl |
| I-794 | NH₂ | 2,4-dichloro-benzyl |
| I-795 | NH₂ | 2,5-dichloro-benzyl |
| I-796 | NH₂ | 2,6-dichloro-benzyl |
| I-797 | NH₂ | 2-fluoro, 4-bromo- |
| | | benzyl |
| I-798 | NH₂ | 2-methyl,4-tert- |
| | | butyl-benzyl |
| I-799 | NH₂ | 2-chloro, |
| | | 6-methanesulfanyl- |
| | | benzyl |
| I-800 | NH₂ | 2-chloro, |
| | | 6-methanesulfinyl- |
| | | benzyl |
| I-801 | NH₂ | 2-methanesulfanyl, |
| | | 5-nitro-benzyl |
| I-802 | NH₂ | 2-methanesulfinyl, |
| | | 5-nitro-benzyl |
| I-803 | NH₂ | 3-amino-benzyl |
| I-804 | NH₂ | 3-bromo-benzyl |
| I-805 | NH₂ | 3-chloro-benzyl |
| I-806 | NH₂ | 3-fluoro-benzyl |
| I-807 | NH₂ | 3-cyano-benzyl |
| I-808 | NH₂ | 3-hydroxy-benzyl |
| I-809 | NH₂ | 3-methoxy-benzyl |
| I-810 | NH₂ | 3-methyl-benzyl |
| I-811 | NH₂ | 3-nitro-benzyl |
| I-812 | NH₂ | 3-benzyloxy-benzyl |
| I-813 | NH₂ | 3-trifluoromethyl- |
| | | benzyl |
| I-814 | NH₂ | 3-trifluoromethoxy- |
| | | benzyl |
| I-815 | NH₂ | 3,5-dichloro-benzyl |
| I-816 | NH₂ | 3,5-di-tert-butyl-4- |
| | | hydroxy-benzyl |
| I-817 | NH₂ | 3-bromo, 4-fluoro- |
| | | benzyl |
| I-818 | NH₂ | 3-bromo, |
| | | 6-fluoro-benzyl |
| I-819 | NH₂ | 3-fluoro, |
| | | 6-methanesulfinyl- |
| | | benzyl |
| I-820 | NH₂ | 3-fluoro, 6- |
| | | methanesulfanyl- |
| | | benzyl |
| I-821 | NH₂ | 3-chloro, |
| | | 6-methanesulfinyl- |
| | | benzyl |
| I-822 | NH₂ | 3-chloro, 6- |
| | | methanesulfanyl- |
| | | benzyl |
| I-823 | NH₂ | 4-amino-benzyl |
| I-824 | NH₂ | 4-benzyloxy-benzyl |
| I-825 | NH₂ | 4-bromo-benzyl |
| I-826 | NH₂ | 4-chloro-benzyl |
| I-827 | NH₂ | 4-fluoro-benzyl |
| I-828 | NH₂ | 4-cyano-benzyl |
| I-829 | NH₂ | 4-acetylamino- |
| | | benzyl |
| I-830 | NH₂ | 4-dimethylamino- |
| | | benzyl |
| I-831 | NH₂ | 4-hydroxy-benzyl |
| I-832 | NH₂ | 4-methyl-benzyl |
| I-833 | NH₂ | 4-methoxy-benzyl |
| I-834 | NH₂ | 4-methoxycarbonyl- |
| | | benzyl |
| I-835 | NH₂ | 4-methanesulfanyl- |
| | | benzyl |
| I-836 | NH₂ | 4-methanesulfinyl- |
| | | benzyl |
| I-837 | NH₂ | 4-N,N-dimethyl- |
| | | amino-benzyl |
| I-838 | NH₂ | 4-nitro-benzyl |
| I-839 | NH₂ | 4-tert-butyl-benzyl |
| I-840 | NH₂ | 4-trifluoromethyl- |
| | | benzyl |
| I-841 | NH₂ | 4-trifluoromethoxy- |
| | | benzyl |
| I-842 | NH₂ | 2,4,6-trimethoxy- |
| | | benzyl |
| I-843 | NH₂ | 2,3,4,5,6- |
| | | pentamethyl-benzyl |
| I-844 | NH₂ | 2-(4-fluoro-phenyl)- |
| | | 4-methyl-pentyl |
| I-845 | NH₂ | 2-[1-phenyl-meth- |
| | | (E)-ylidene]-heptyl |
| I-846 | NH₂ | 1-phenyl-prop-2- |
| | | ynyl |
| I-847 | NH₂ | 1-phenyl-3- |
| | | trimethylsilanyl- |
| | | prop-2-ynyl |
| I-848 | NH₂ | 4-ethoxycarbonyl-1- |
| | | phenyl-but-2-ynyl |
| I-849 | NH₂ | 4-methoxy-1- |
| | | phenyl-but-2-ynyl |
| I-850 | NH₂ | 4,4-dimethyl-1- |
| | | phenyl-pent-2-ynyl |
| I-851 | NH₂ | 1-phenyl-hex-2-ynyl |
| I-852 | NH₂ | 1-(4-chloro-phenyl)- |
| | | hex-2-ynyl |
| I-853 | NH₂ | 1-(4-methoxy- |
| | | phenyl)-hex-2-ynyl |
| I-854 | NH₂ | phenyl |
| I-855 | NH₂ | 2-chlorophenyl |
| I-856 | NH₂ | 2-fluorophenyl |
| I-857 | NH₂ | 2-trifluoromethyl- |
| | | phenyl |
| I-858 | NH₂ | 2-methylphenyl |
| I-859 | NH₂ | 2-methoxyphenyl |
| I-860 | NH₂ | 3-chlorophenyl |
| I-861 | NH₂ | 3-fluorophenyl |
| I-862 | NH₂ | 3-trifluoromethyl- |
| | | phenyl |
| I-863 | NH₂ | 3-methoxyphenyl |
| I-864 | NH₂ | 3-methylphenyl |
| I-865 | NH₂ | 4-chlorophenyl |
| I-866 | NH₂ | 4-fluorophenyl |
| I-867 | NH₂ | 4-methylphenyl |
| I-868 | NH₂ | 4-methoxyphenyl |
| I-869 | NH₂ | 4-trifluoromethyl- |
| | | phenyl |
| I-870 | NH₂ | 9H-fluoren-9-yl |
| I-871 | NHC(O)CH₃ | ethyl |
| I-872 | NHC(O)CH₃ | n-propyl |
| I-873 | NHC(O)CH₃ | 1-methyl-ethyl |
| I-874 | NHC(O)CH₃ | n-butyl |
| I-875 | NHC(O)CH₃ | 2-methyl-propyl |
| I-876 | NHC(O)CH₃ | 3-methyl-butyl |
| I-877 | NHC(O)CH₃ | 2,2-dimethyl-propyl |
| I-878 | NHC(O)CH₃ | 3,3-dimethyl-butyl |
| I-879 | NHC(O)CH₃ | 4,4-dimethyl-pentyl |
| I-880 | NHC(O)CH₃ | 2-ethyl-hexyl |
| I-881 | NHC(O)CH₃ | 3,5,5-trimethyl-hexyl |
| I-882 | NHC(O)CH₃ | 2-chloro-ethyl |
| I-883 | NHC(O)CH₃ | 2-tert-butoxy-propyl |
| I-884 | NHC(O)CH₃ | 2,3-diacetoxy-propyl |
| I-885 | NHC(O)CH₃ | 3-methoxycarbonyl- |
| | | propyl |
| I-886 | NHC(O)CH₃ | 3-methylsulfanyl- |
| | | propyl |
| I-887 | NHC(O)CH₃ | 2,3-dihydroxy- |
| | | propyl |
| I-888 | NHC(O)CH₃ | 2-hydroxy-butyl |
| I-889 | NHC(O)CH₃ | 2-acetoxy-butyl |
| I-890 | NHC(O)CH₃ | 2-benzyloxy-butyl |
| I-891 | NHC(O)CH₃ | 2-dimethylamino-2- |
| | | methyl-propyl |
| I-892 | NHC(O)CH₃ | 1-phenylethynyl- |
| | | butyl |
| I-893 | NHC(O)CH₃ | allyl |
| I-894 | NHC(O)CH₃ | 2,6-dimethyl-hept-5- |
| | | enyl |
| I-895 | NHC(O)CH₃ | (Z)-dec-4-enyl |
| I-896 | NHC(O)CH₃ | 2-(2,6,6-trimethyl- |
| | | cyclohex-1-enyl)- |
| | | ethyl |
| I-897 | NHC(O)CH₃ | 1-naphtalene- |
| | | methyl |
| I-898 | NHC(O)CH₃ | 2-naphtalene- |
| | | methyl |
| I-899 | NHC(O)CH₃ | phenethyl |
| I-900 | NHC(O)CH₃ | 2-phenyl-propyl |
| I-901 | NHC(O)CH₃ | 3-phenyl-propyl |
| I-902 | NHC(O)CH₃ | 4-benzo[1,3]dioxol- |
| | | 5-yl-butyl |
| I-903 | NHC(O)CH₃ | cyclopropyl-methyl |
| I-904 | NHC(O)CH₃ | 1-(4-chloro-phenyl)- |
| | | 1H-pyrrol-3-ylmethyl |
| I-905 | NHC(O)CH₃ | furan-2-ylmethyl |
| I-906 | NHC(O)CH₃ | furan-3-ylmethyl |
| I-907 | NHC(O)CH₃ | 3-dimethyl- |
| | | aminomethyl- |
| | | tetrahydro-furan-3- |
| | | ylmethyl |
| I-908 | NHC(O)CH₃ | 2,5-dihydro- |
| | | thiophen-3-ylmethyl |
| I-909 | NHC(O)CH₃ | 1-tert-butyl-1H- |
| | | pyrazol-4-ylmethyl |
| I-910 | NHC(O)CH₃ | 5-methyl-1H- |
| | | imidazol-4-ylmethyl |
| I-911 | NHC(O)CH₃ | 2-chloro-thiazol-5- |
| | | ylmethyl |
| I-912 | NHC(O)CH₃ | 3-isobutyl-isoxazol- |
| | | 5-ylmethyl |
| I-913 | NHC(O)CH₃ | 1-methyl-1H-pyrrol- |
| | | 2-ylmethyl |
| I-914 | NHC(O)CH₃ | tetrahydro-pyran-3- |
| | | ylmethyl |
| I-915 | NHC(O)CH₃ | tetrahydro-pyran-4- |
| | | ylmethyl |
| I-916 | NHC(O)CH₃ | tetrahydro- |
| | | thiopyran-3-ylmethyl |
| I-917 | NHC(O)CH₃ | thiophen-2-ylmethyl |
| I-918 | NHC(O)CH₃ | pyridin-2-ylmethyl |
| I-919 | NHC(O)CH₃ | pyridin-3-ylmethyl |
| I-920 | NHC(O)CH₃ | pyridin-4-ylmethyl |
| I-921 | NHC(O)CH₃ | 6-chloro-pyridin-3- |
| | | ylmethyl |
| I-922 | NHC(O)CH₃ | 1H-indol-3-ylmethyl |
| I-923 | NHC(O)CH₃ | 3-chloro-1H-indol-2- |
| | | ylmethyl |
| I-924 | NHC(O)CH₃ | 2-(tetrahydro-pyran- |
| | | 4-yl)-ethyl |
| I-925 | NHC(O)CH₃ | 2,2-dimethyl-3- |
| | | pyridin-3-yl-propyl |
| I-926 | NHC(O)CH₃ | benzyl |
| I-927 | NHC(O)CH₃ | 2-amino-benzyl |
| I-928 | NHC(O)CH₃ | 2-bromo-benzyl |
| I-929 | NHC(O)CH₃ | 2-fluoro-benzyl |
| I-930 | NHC(O)CH₃ | 2-chloro-benzyl |
| I-931 | NHC(O)CH₃ | 2-cyano-benzyl |
| I-932 | NHC(O)CH₃ | 2-hydroxy-benzyl |
| I-933 | NHC(O)CH₃ | 2-methyl-benzyl |
| I-934 | NHC(O)CH₃ | 2-methoxy-benzyl |
| I-935 | NHC(O)CH₃ | 2-trifluoromethyl- |
| | | benzyl |
| I-936 | NHC(O)CH₃ | 2-trifluoromethoxy- |
| | | benzyl |
| I-937 | NHC(O)CH₃ | 2-diphenyl- |
| | | phosphino-benzyl |
| I-938 | NHC(O)CH₃ | 2-benzyloxy-benzyl |
| I-939 | NHC(O)CH₃ | 2,4-dichloro-benzyl |
| I-940 | NHC(O)CH₃ | 2,5-dichloro-benzyl |
| I-941 | NHC(O)CH₃ | 2,6-dichloro-benzyl |
| I-942 | NHC(O)CH₃ | 2-fluoro, 4-bromo- |
| | | benzyl |
| I-943 | NHC(O)CH₃ | 2-methyl,4-tert- |
| | | butyl-benzyl |
| I-944 | NHC(O)CH₃ | 2-chloro, |
| | | 6-methanesulfanyl- |
| | | benzyl |
| I-945 | NHC(O)CH₃ | 2-chloro, |
| | | 6-methanesulfinyl- |
| | | benzyl |
| I-946 | NHC(O)CH₃ | 2-methanesulfanyl, |
| | | 5-nitro-benzyl |
| I-947 | NHC(O)CH₃ | 2-methanesulfinyl, |
| | | 5-nitro-benzyl |
| I-948 | NHC(O)CH₃ | 3-amino-benzyl |
| I-949 | NHC(O)CH₃ | 3-bromo-benzyl |
| I-950 | NHC(O)CH₃ | 3-chloro-benzyl |
| I-951 | NHC(O)CH₃ | 3-fluoro-benzyl |
| I-952 | NHC(O)CH₃ | 3-cyano-benzyl |
| I-953 | NHC(O)CH₃ | 3-hydroxy-benzyl |
| I-954 | NHC(O)CH₃ | 3-methoxy-benzyl |
| I-955 | NHC(O)CH₃ | 3-methyl-benzyl |
| I-956 | NHC(O)CH₃ | 3-nitro-benzyl |
| I-957 | NHC(O)CH₃ | 3-benzyloxy-benzyl |
| I-958 | NHC(O)CH₃ | 3-trifluoromethyl- |
| | | benzyl |
| I-959 | NHC(O)CH₃ | 3-trifluoromethoxy- |
| | | benzyl |
| I-960 | NHC(O)CH₃ | 3,5-dichloro-benzyl |
| I-961 | NHC(O)CH₃ | 3,5-di-tert-butyl-4- |
| | | hydroxy-benzyl |
| I-962 | NHC(O)CH₃ | 3-bromo, 4-fluoro- |
| | | benzyl |
| I-963 | NHC(O)CH₃ | 3-bromo, |
| | | 6-fluoro-benzyl |
| I-964 | NHC(O)CH₃ | 3-fluoro, |
| | | 6-methanesulfinyl- |
| | | benzyl |
| I-965 | NHC(O)CH₃ | 3-fluoro, 6- |
| | | methanesulfanyl- |
| | | benzyl |
| I-966 | NHC(O)CH₃ | 3-chloro, |
| | | 6-methanesulfinyl- |
| | | benzyl |
| I-967 | NHC(O)CH₃ | 3-chloro, 6- |
| | | methanesulfanyl- |
| | | benzyl |
| I-968 | NHC(O)CH₃ | 4-amino-benzyl |
| I-969 | NHC(O)CH₃ | 4-benzyloxy-benzyl |
| I-970 | NHC(O)CH₃ | 4-bromo-benzyl |
| I-971 | NHC(O)CH₃ | 4-chloro-benzyl |
| I-972 | NHC(O)CH₃ | 4-fluoro-benzyl |
| I-973 | NHC(O)CH₃ | 4-cyano-benzyl |
| I-974 | NHC(O)CH₃ | 4-acetylamino- |
| | | benzyl |
| I-975 | NHC(O)CH₃ | 4-dimethylamino- |
| | | benzyl |
| I-976 | NHC(O)CH₃ | 4-hydroxy-benzyl |
| I-977 | NHC(O)CH₃ | 4-methyl-benzyl |
| I-978 | NHC(O)CH₃ | 4-methoxy-benzyl |
| I-979 | NHC(O)CH₃ | 4-methoxycarbonyl- |
| | | benzyl |
| I-980 | NHC(O)CH₃ | 4-methanesulfanyl- |
| | | benzyl |
| I-981 | NHC(O)CH₃ | 4-methanesulfinyl- |
| | | benzyl |
| I-982 | NHC(O)CH₃ | 4-N,N-dimethyl- |
| | | amino-benzyl |
| I-983 | NHC(O)CH₃ | 4-nitro-benzyl |
| I-984 | NHC(O)CH₃ | 4-tert-butyl-benzyl |
| I-985 | NHC(O)CH₃ | 4-trifluoromethyl- |
| | | benzyl |
| I-986 | NHC(O)CH₃ | 4-trifluoromethoxy- |
| | | benzyl |
| I-987 | NHC(O)CH₃ | 2,4,6-trimethoxy- |
| | | benzyl |
| I-988 | NHC(O)CH₃ | 2,3,4,5,6- |
| | | pentamethyl-benzyl |
| I-989 | NHC(O)CH₃ | 2-(4-fluoro-phenyl)- |
| | | 4-methyl-pentyl |
| I-990 | NHC(O)CH₃ | 2-[1-phenyl-meth- |
| | | (E)-ylidene]-heptyl |
| I-991 | NHC(O)CH₃ | 1-phenyl-prop-2- |
| | | ynyl |
| I-992 | NHC(O)CH₃ | 1-phenyl-3- |
| | | trimethylsilanyl- |
| | | prop-2-ynyl |
| I-993 | NHC(O)CH₃ | 4-ethoxycarbonyl-1- |
| | | phenyl-but-2-ynyl |
| I-994 | NHC(O)CH₃ | 4-methoxy-1- |
| | | phenyl-but-2-ynyl |
| I-995 | NHC(O)CH₃ | 4,4-dimethyl-1- |
| | | phenyl-pent-2-ynyl |
| I-996 | NHC(O)CH₃ | 1-phenyl-hex-2-ynyl |
| I-997 | NHC(O)CH₃ | 1-(4-chloro-phenyl)- |
| | | hex-2-ynyl |
| I-998 | NHC(O)CH₃ | 1-(4-methoxy- |
| | | phenyl)-hex-2-ynyl |
| I-999 | NHC(O)CH₃ | phenyl |
| I-1000 | NHC(O)CH₃ | 2-chlorophenyl |
| I-1001 | NHC(O)CH₃ | 2-fluorophenyl |
| I-1002 | NHC(O)CH₃ | 2-trifluoromethyl- |
| | | phenyl |
| I-1003 | NHC(O)CH₃ | 2-methylphenyl |
| I-1004 | NHC(O)CH₃ | 2-methoxyphenyl |
| I-1005 | NHC(O)CH₃ | 3-chlorophenyl |
| I-1006 | NHC(O)CH₃ | 3-fluorophenyl |
| I-1007 | NHC(O)CH₃ | 3-trifluoromethyl- |
| | | phenyl |
| I-1008 | NHC(O)CH₃ | 3-methoxyphenyl |
| I-1009 | NHC(O)CH₃ | 3-methylphenyl |
| I-1010 | NHC(O)CH₃ | 4-chlorophenyl |
| I-1011 | NHC(O)CH₃ | 4-fluorophenyl |
| I-1012 | NHC(O)CH₃ | 4-methylphenyl |
| I-1013 | NHC(O)CH₃ | 4-methoxyphenyl |
| I-1014 | NHC(O)CH₃ | 4-trifluoromethyl- |
| | | phenyl |
| I-1015 | NHC(O)CH₃ | 9H-fluoren-9-yl |
| I-1016 | CH═CH₂ | ethyl |
| I-1017 | CH═CH₂ | n-propyl |
| I-1018 | CH═CH₂ | 1-methyl-ethyl |
| I-1019 | CH═CH₂ | n-butyl |
| I-1020 | CH═CH₂ | 2-methyl-propyl |
| I-1021 | CH═CH₂ | 3-methyl-butyl |
| I-1022 | CH═CH₂ | 2,2-dimethyl-propyl |
| I-1023 | CH═CH₂ | 3,3-dimethyl-butyl |
| I-1024 | CH═CH₂ | 4,4-dimethyl-pentyl |
| I-1025 | CH═CH₂ | 2-ethyl-hexyl |
| I-1026 | CH═CH₂ | 3,5,5-trimethyl-hexyl |
| I-1027 | CH═CH₂ | 2-chloro-ethyl |
| I-1028 | CH═CH₂ | 2-tert-butoxy-propyl |
| I-1029 | CH═CH₂ | 2,3-diacetoxy-propyl |
| I-1030 | CH═CH₂ | 3-methoxycarbonyl- |
| | | propyl |
| I-1031 | CH═CH₂ | 3-methylsulfanyl- |
| | | propyl |
| I-1032 | CH═CH₂ | 2,3-dihydroxy- |
| | | propyl |
| I-1033 | CH═CH₂ | 2-hydroxy-butyl |
| I-1034 | CH═CH₂ | 2-acetoxy-butyl |
| I-1035 | CH═CH₂ | 2-benzyloxy-butyl |
| I-1036 | CH═CH₂ | 2-dimethylamino-2- |
| | | methyl-propyl |
| I-1037 | CH═CH₂ | 1-phenylethynyl- |
| | | butyl |
| I-1038 | CH═CH₂ | allyl |
| I-1039 | CH═CH₂ | 2,6-dimethyl-hept-5- |
| | | enyl |
| I-1040 | CH═CH₂ | (Z)-dec-4-enyl |
| I-1041 | CH═CH₂ | 2-(2,6,6-trimethyl- |
| | | cyclohex-1-enyl)- |
| | | ethyl |
| I-1042 | CH═CH₂ | 1-naphtalene- |
| | | methyl |
| I-1043 | CH═CH₂ | 2-naphtalene- |
| | | methyl |
| I-1044 | CH═CH₂ | phenethyl |
| I-1045 | CH═CH₂ | 2-phenyl-propyl |
| I-1046 | CH═CH₂ | 3-phenyl-propyl |
| I-1047 | CH═CH₂ | 4-benzo[1,3]dioxol- |
| | | 5-yl-butyl |
| I-1048 | CH═CH₂ | cyclopropyl-methyl |
| I-1049 | CH═CH₂ | 1-(4-chloro-phenyl)- |
| | | 1H-pyrrol-3-ylmethyl |
| I-1050 | CH═CH₂ | furan-2-ylmethyl |
| I-1051 | CH═CH₂ | furan-3-ylmethyl |
| I-1052 | CH═CH₂ | 3-dimethyl- |
| | | aminomethyl- |
| | | tetrahydro-furan-3- |
| | | ylmethyl |
| I-1053 | CH═CH₂ | 2,5-dihydro- |
| | | thiophen-3-ylmethyl |
| I-1054 | CH═CH₂ | 1-tert-butyl-1H- |
| | | pyrazol-4-ylmethyl |
| I-1055 | CH═CH₂ | 5-methyl-1H- |
| | | imidazol-4-ylmethyl |
| I-1056 | CH═CH₂ | 2-chloro-thiazol-5- |
| | | ylmethyl |
| I-1057 | CH═CH₂ | 3-isobutyl-isoxazol- |
| | | 5-ylmethyl |
| I-1058 | CH═CH₂ | 1-methyl-1H-pyrrol- |
| | | 2-ylmethyl |
| I-1059 | CH═CH₂ | tetrahydro-pyran-3- |
| | | ylmethyl |
| I-1060 | CH═CH₂ | tetrahydro-pyran-4- |
| | | ylmethyl |
| I-1061 | CH═CH₂ | tetrahydro- |
| | | thiopyran-3-ylmethyl |
| I-1062 | CH═CH₂ | thiophen-2-ylmethyl |
| I-1063 | CH═CH₂ | pyridin-2-ylmethyl |
| I-1064 | CH═CH₂ | pyridin-3-ylmethyl |
| I-1065 | CH═CH₂ | pyridin-4-ylmethyl |
| I-1066 | CH═CH₂ | 6-chloro-pyridin-3- |
| | | ylmethyl |
| I-1067 | CH═CH₂ | 1H-indol-3-ylmethyl |
| I-1068 | CH═CH₂ | 3-chloro-1H-indol-2- |
| | | ylmethyl |
| I-1069 | CH═CH₂ | 2-(tetrahydro-pyran- |
| | | 4-yl)-ethyl |
| I-1070 | CH═CH₂ | 2,2-dimethyl-3- |
| | | pyridin-3-yl-propyl |
| I-1071 | CH═CH₂ | benzyl |
| I-1072 | CH═CH₂ | 2-amino-benzyl |
| I-1073 | CH═CH₂ | 2-bromo-benzyl |
| I-1074 | CH═CH₂ | 2-fluoro-benzyl |
| I-1075 | CH═CH₂ | 2-chloro-benzyl |
| I-1076 | CH═CH₂ | 2-cyano-benzyl |
| I-1077 | CH═CH₂ | 2-hydroxy-benzyl |
| I-1078 | CH═CH₂ | 2-methyl-benzyl |
| I-1079 | CH═CH₂ | 2-methoxy-benzyl |
| I-1080 | CH═CH₂ | 2-trifluoromethyl- |
| | | benzyl |
| I-1081 | CH═CH₂ | 2-trifluoromethoxy- |
| | | benzyl |
| I-1082 | CH═CH₂ | 2-diphenyl- |
| | | phosphino-benzyl |
| I-1083 | CH═CH₂ | 2-benzyloxy-benzyl |
| I-1084 | CH═CH₂ | 2,4-dichloro-benzyl |
| I-1085 | CH═CH₂ | 2,5-dichloro-benzyl |
| I-1086 | CH═CH₂ | 2,6-dichloro-benzyl |
| I-1087 | CH═CH₂ | 2-fluoro, 4-bromo- |
| | | benzyl |
| I-1088 | CH═CH₂ | 2-methyl,4-tert- |
| | | butyl-benzyl |
| I-1089 | CH═CH₂ | 2-chloro, |
| | | 6-methanesulfanyl- |
| | | benzyl |
| I-1090 | CH═CH₂ | 2-chloro, |
| | | 6-methanesulfinyl- |
| | | benzyl |
| I-1091 | CH═CH₂ | 2-methanesulfanyl, |
| | | 5-nitro-benzyl |
| I-1092 | CH═CH₂ | 2-methanesulfinyl, |
| | | 5-nitro-benzyl |
| I-1093 | CH═CH₂ | 3-amino-benzyl |
| I-1094 | CH═CH₂ | 3-bromo-benzyl |
| I-1095 | CH═CH₂ | 3-chloro-benzyl |
| I-1096 | CH═CH₂ | 3-fluoro-benzyl |
| I-1097 | CH═CH₂ | 3-cyano-benzyl |
| I-1098 | CH═CH₂ | 3-hydroxy-benzyl |
| I-1099 | CH═CH₂ | 3-methoxy-benzyl |
| I-1100 | CH═CH₂ | 3-methyl-benzyl |
| I-1101 | CH═CH₂ | 3-nitro-benzyl |
| I-1102 | CH═CH₂ | 3-benzyloxy-benzyl |
| I-1103 | CH═CH₂ | 3-trifluoromethyl- |
| | | benzyl |
| I-1104 | CH═CH₂ | 3-trifluoromethoxy- |
| | | benzyl |
| I-1105 | CH═CH₂ | 3,5-dichloro-benzyl |
| I-1106 | CH═CH₂ | 3,5-di-tert-butyl-4- |
| | | hydroxy-benzyl |
| I-1107 | CH═CH₂ | 3-bromo, 4-fluoro- |
| | | benzyl |
| I-1108 | CH═CH₂ | 3-bromo, |
| | | 6-fluoro-benzyl |
| I-1109 | CH═CH₂ | 3-fluoro, |
| | | 6-methanesulfinyl- |
| | | benzyl |
| I-1110 | CH═CH₂ | 3-fluoro, 6- |
| | | methanesulfanyl- |
| | | benzyl |
| I-1111 | CH═CH₂ | 3-chloro, |
| | | 6-methanesulfinyl- |
| | | benzyl |
| I-1112 | CH═CH₂ | 3-chloro, 6- |
| | | methanesulfanyl- |
| | | benzyl |
| I-1113 | CH═CH₂ | 4-amino-benzyl |
| I-1114 | CH═CH₂ | 4-benzyloxy-benzyl |
| I-1115 | CH═CH₂ | 4-bromo-benzyl |
| I-1116 | CH═CH₂ | 4-chloro-benzyl |
| I-1117 | CH═CH₂ | 4-fluoro-benzyl |
| I-1118 | CH═CH₂ | 4-cyano-benzyl |
| I-1119 | CH═CH₂ | 4-acetylamino- |
| | | benzyl |
| I-1120 | CH═CH₂ | 4-dimethylamino- |
| | | benzyl |
| I-1121 | CH═CH₂ | 4-hydroxy-benzyl |
| I-1122 | CH═CH₂ | 4-methyl-benzyl |
| I-1123 | CH═CH₂ | 4-methoxy-benzyl |
| I-1124 | CH═CH₂ | 4-methoxycarbonyl- |
| | | benzyl |
| I-1125 | CH═CH₂ | 4-methanesulfanyl- |
| | | benzyl |
| I-1126 | CH═CH₂ | 4-methanesulfinyl- |
| | | benzyl |
| I-1127 | CH═CH₂ | 4-N,N-dimethyl- |
| | | amino-benzyl |
| I-1128 | CH═CH₂ | 4-nitro-benzyl |
| I-1129 | CH═CH₂ | 4-tert-butyl-benzyl |
| I-1130 | CH═CH₂ | 4-trifluoromethyl- |
| | | benzyl |
| I-1131 | CH═CH₂ | 4-trifluoromethoxy- |
| | | benzyl |
| I-1132 | CH═CH₂ | 2,4,6-trimethoxy- |
| | | benzyl |
| I-1133 | CH═CH₂ | 2,3,4,5,6- |
| | | pentamethyl-benzyl |
| I-1134 | CH═CH₂ | 2-(4-fluoro-phenyl)- |
| | | 4-methyl-pentyl |
| I-1135 | CH═CH₂ | 2-[1-phenyl-meth- |
| | | (E)-ylidene]-heptyl |
| I-1136 | CH═CH₂ | 1-phenyl-prop-2- |
| | | ynyl |
| I-1137 | CH═CH₂ | 1-phenyl-3- |
| | | trimethylsilanyl- |
| | | prop-2-ynyl |
| I-1138 | CH═CH₂ | 4-ethoxycarbonyl-1- |
| | | phenyl-but-2-ynyl |
| I-1139 | CH═CH₂ | 4-methoxy-1-phenyl- |
| | | but-2-ynyl |
| I-1140 | CH═CH₂ | 4,4-dimethyl-1- |
| | | phenyl-pent-2-ynyl |
| I-1141 | CH═CH₂ | 1-phenyl-hex-2-ynyl |
| I-1142 | CH═CH₂ | 1-(4-chloro-phenyl)- |
| | | hex-2-ynyl |
| I-1143 | CH═CH₂ | 1-(4-methoxy- |
| | | phenyl)-hex-2-ynyl |
| I-1144 | CH═CH₂ | phenyl |
| I-1145 | CH═CH₂ | 2-chlorophenyl |
| I-1146 | CH═CH₂ | 2-fluorophenyl |
| I-1147 | CH═CH₂ | 2-trifluoromethyl- |
| | | phenyl |
| I-1148 | CH═CH₂ | 2-methylphenyl |
| I-1149 | CH═CH₂ | 2-methoxyphenyl |
| I-1150 | CH═CH₂ | 3-chlorophenyl |
| I-1151 | CH═CH₂ | 3-fluorophenyl |
| I-1152 | CH═CH₂ | 3-trifluoromethyl- |
| | | phenyl |
| I-1153 | CH═CH₂ | 3-methoxyphenyl |
| I-1154 | CH═CH₂ | 3-methylphenyl |
| I-1155 | CH═CH₂ | 4-chlorophenyl |
| I-1156 | CH═CH₂ | 4-fluorophenyl |
| I-1157 | CH═CH₂ | 4-methylphenyl |
| I-1158 | CH═CH₂ | 4-methoxyphenyl |
| I-1159 | CH═CH₂ | 4-trifluoromethyl- |
| | | phenyl |
| I-1160 | CH═CH₂ | 9H-fluoren-9-yl |
| I-1161 | ethynyl | ethyl |
| I-1162 | ethynyl | n-propyl |
| I-1163 | ethynyl | 1-methyl-ethyl |
| I-1164 | ethynyl | n-butyl |
| I-1165 | ethynyl | 2-methyl-propyl |
| I-1166 | ethynyl | 3-methyl-butyl |
| I-1167 | ethynyl | 2,2-dimethyl-propyl |
| I-1168 | ethynyl | 3,3-dimethyl-butyl |
| I-1169 | ethynyl | 4,4-dimethyl-pentyl |
| I-1170 | ethynyl | 2-ethyl-hexyl |
| I-1171 | ethynyl | 3,5,5-trimethyl-hexyl |
| I-1172 | ethynyl | 2-chloro-ethyl |
| I-1173 | ethynyl | 2-tert-butoxy-propyl |
| I-1174 | ethynyl | 2,3-diacetoxy-propyl |
| I-1175 | ethynyl | 3-methoxycarbonyl- |
| | | propyl |
| I-1176 | ethynyl | 3-methylsulfanyl- |
| | | propyl |
| I-1177 | ethynyl | 2,3-dihydroxy- |
| | | propyl |
| I-1178 | ethynyl | 2-hydroxy-butyl |
| I-1179 | ethynyl | 2-acetoxy-butyl |
| I-1180 | ethynyl | 2-benzyloxy-butyl |
| I-1181 | ethynyl | 2-dimethylamino-2- |
| | | methyl-propyl |
| I-1182 | ethynyl | 1-phenylethynyl- |
| | | butyl |
| I-1183 | ethynyl | allyl |
| I-1184 | ethynyl | 2,6-dimethyl-hept-5- |
| | | enyl |
| I-1185 | ethynyl | (Z)-dec-4-enyl |
| I-1186 | ethynyl | 2-(2,6,6-trimethyl- |
| | | cyclohex-1-enyl)- |
| | | ethyl |
| I-1187 | ethynyl | 1-naphtalene- |
| | | methyl |
| I-1188 | ethynyl | 2-naphtalene- |
| | | methyl |
| I-1189 | ethynyl | phenethyl |
| I-1190 | ethynyl | 2-phenyl-propyl |
| I-1191 | ethynyl | 3-phenyl-propyl |
| I-1192 | ethynyl | 4-benzo[1,3]dioxol- |
| | | 5-yl-butyl |
| I-1193 | ethynyl | cyclopropyl-methyl |
| I-1194 | ethynyl | 1-(4-chloro-phenyl)- |
| | | 1H-pyrrol-3-ylmethyl |
| I-1195 | ethynyl | furan-2-ylmethyl |
| I-1196 | ethynyl | furan-3-ylmethyl |
| I-1197 | ethynyl | 3-dimethyl- |
| | | aminomethyl- |
| | | tetrahydro-furan-3- |
| | | ylmethyl |
| I-1198 | ethynyl | 2,5-dihydro- |
| | | thiophen-3-ylmethyl |
| I-1199 | ethynyl | 1-tert-butyl-1H- |
| | | pyrazol-4-ylmethyl |
| I-1200 | ethynyl | 5-methyl-1H- |
| | | imidazol-4-ylmethyl |
| I-1201 | ethynyl | 2-chloro-thiazol-5- |
| | | ylmethyl |
| I-1202 | ethynyl | 3-isobutyl-isoxazol- |
| | | 5-ylmethyl |
| I-1203 | ethynyl | 1-methyl-1H-pyrrol- |
| | | 2-ylmethyl |
| I-1204 | ethynyl | tetrahydro-pyran-3- |
| | | ylmethyl |
| I-1205 | ethynyl | tetrahydro-pyran-4- |
| | | ylmethyl |
| I-1206 | ethynyl | tetrahydro- |
| | | thiopyran-3-ylmethyl |
| I-1207 | ethynyl | thiophen-2-ylmethyl |
| I-1208 | ethynyl | pyridin-2-ylmethyl |
| I-1209 | ethynyl | pyridin-3-ylmethyl |
| I-1210 | ethynyl | pyridin-4-ylmethyl |
| I-1211 | ethynyl | 6-chloro-pyridin-3- |
| | | ylmethyl |
| I-1212 | ethynyl | 1H-indol-3-ylmethyl |
| I-1213 | ethynyl | 3-chloro-1H-indol-2- |
| | | ylmethyl |
| I-1214 | ethynyl | 2-(tetrahydro-pyran- |
| | | 4-yl)-ethyl |
| I-1215 | ethynyl | 2,2-dimethyl-3- |
| | | pyridin-3-yl-propyl |
| I-1216 | ethynyl | benzyl |
| I-1217 | ethynyl | 2-amino-benzyl |
| I-1218 | ethynyl | 2-bromo-benzyl |
| I-1219 | ethynyl | 2-fluoro-benzyl |
| I-1220 | ethynyl | 2-chloro-benzyl |
| I-1221 | ethynyl | 2-cyano-benzyl |
| I-1222 | ethynyl | 2-hydroxy-benzyl |
| I-1223 | ethynyl | 2-methyl-benzyl |
| I-1224 | ethynyl | 2-methoxy-benzyl |
| I-1225 | ethynyl | 2-trifluoromethyl- |
| | | benzyl |
| I-1226 | ethynyl | 2-trifluoromethoxy- |
| | | benzyl |
| I-1227 | ethynyl | 2-diphenyl- |
| | | phosphino-benzyl |
| I-1228 | ethynyl | 2-benzyloxy-benzyl |
| I-1229 | ethynyl | 2,4-dichloro-benzyl |
| I-1230 | ethynyl | 2,5-dichloro-benzyl |
| I-1231 | ethynyl | 2,6-dichloro-benzyl |
| I-1232 | ethynyl | 2-fluoro, 4-bromo- |
| | | benzyl |
| I-1233 | ethynyl | 2-methyl,4-tert- |
| | | butyl-benzyl |
| I-1234 | ethynyl | 2-chloro, |
| | | 6-methanesulfanyl- |
| | | benzyl |
| I-1235 | ethynyl | 2-chloro, |
| | | 6-methanesulfinyl- |
| | | benzyl |
| I-1236 | ethynyl | 2-methanesulfanyl, |
| | | 5-nitro-benzyl |
| I-1237 | ethynyl | 2-methanesulfinyl, |
| | | 5-nitro-benzyl |
| I-1238 | ethynyl | 3-amino-benzyl |
| I-1239 | ethynyl | 3-bromo-benzyl |
| I-1240 | ethynyl | 3-chloro-benzyl |
| I-1241 | ethynyl | 3-fluoro-benzyl |
| I-1242 | ethynyl | 3-cyano-benzyl |
| I-1243 | ethynyl | 3-hydroxy-benzyl |
| I-1244 | ethynyl | 3-methoxy-benzyl |
| I-1245 | ethynyl | 3-methyl-benzyl |
| I-1246 | ethynyl | 3-nitro-benzyl |
| I-1247 | ethynyl | 3-benzyloxy-benzyl |
| I-1248 | ethynyl | 3-trifluoromethyl- |
| | | benzyl |
| I-1249 | ethynyl | 3-trifluoromethoxy- |
| | | benzyl |
| I-1250 | ethynyl | 3,5-dichloro-benzyl |
| I-1251 | ethynyl | 3,5-di-tert-butyl-4- |
| | | hydroxy-benzyl |
| I-1252 | ethynyl | 3-bromo, 4-fluoro- |
| | | benzyl |
| I-1253 | ethynyl | 3-bromo, |
| | | 6-fluoro-benzyl |
| I-1254 | ethynyl | 3-fluoro, |
| | | 6-methanesulfinyl- |
| | | benzyl |
| I-1255 | ethynyl | 3-fluoro, 6- |
| | | methanesulfanyl- |
| | | benzyl |
| I-1256 | ethynyl | 3-chloro, |
| | | 6-methanesulfinyl- |
| | | benzyl |
| I-1257 | ethynyl | 3-chloro, 6- |
| | | methanesulfanyl- |
| | | benzyl |
| I-1258 | ethynyl | 4-amino-benzyl |
| I-1259 | ethynyl | 4-benzyloxy-benzyl |
| I-1260 | ethynyl | 4-bromo-benzyl |
| I-1261 | ethynyl | 4-chloro-benzyl |
| I-1262 | ethynyl | 4-fluoro-benzyl |
| I-1263 | ethynyl | 4-cyano-benzyl |
| I-1264 | ethynyl | 4-acetylamino- |
| | | benzyl |
| I-1265 | ethynyl | 4-dimethylamino- |
| | | benzyl |
| I-1266 | ethynyl | 4-hydroxy-benzyl |
| I-1267 | ethynyl | 4-methyl-benzyl |
| I-1268 | ethynyl | 4-methoxy-benzyl |
| I-1269 | ethynyl | 4-methoxycarbonyl- |
| | | benzyl |
| I-1270 | ethynyl | 4-methanesulfanyl- |
| | | benzyl |
| I-1271 | ethynyl | 4-methanesulfinyl- |
| | | benzyl |
| I-1272 | ethynyl | 4-N,N-dimethyl- |
| | | amino-benzyl |
| I-1273 | ethynyl | 4-nitro-benzyl |
| I-1274 | ethynyl | 4-tert-butyl-benzyl |
| I-1275 | ethynyl | 4-trifluoromethyl- |
| | | benzyl |
| I-1276 | ethynyl | 4-trifluoromethoxy- |
| | | benzyl |
| I-1277 | ethynyl | 2,4,6-trimethoxy- |
| | | benzyl |
| I-1278 | ethynyl | 2,3,4,5,6- |
| | | pentamethyl-benzyl |
| I-1279 | ethynyl | 2-(4-fluoro-phenyl)- |
| | | 4-methyl-pentyl |
| I-1280 | ethynyl | 2-[1-phenyl-meth- |
| | | (E)-ylidene]-heptyl |
| I-1281 | ethynyl | 1-phenyl-prop-2- |
| | | ynyl |
| I-1282 | ethynyl | 1-phenyl-3- |
| | | trimethylsilanyl- |
| | | prop-2-ynyl |
| I-1283 | ethynyl | 4-ethoxycarbonyl-1- |
| | | phenyl-but-2-ynyl |
| I-1284 | ethynyl | 4-methoxy-1- |
| | | phenyl-but-2-ynyl |
| I-1285 | ethynyl | 4,4-dimethyl-1- |
| | | phenyl-pent-2-ynyl |
| I-1286 | ethynyl | 1-phenyl-hex-2-ynyl |
| I-1287 | ethynyl | 1-(4-chloro-phenyl)- |
| | | hex-2-ynyl |
| I-1288 | ethynyl | 1-(4-methoxy- |
| | | phenyl)-hex-2-ynyl |
| I-1289 | ethynyl | phenyl |
| I-1290 | ethynyl | 2-chlorophenyl |
| I-1291 | ethynyl | 2-fluorophenyl |
| I-1292 | ethynyl | 2-trifluoromethyl- |
| | | phenyl |
| I-1293 | ethynyl | 2-methylphenyl |
| I-1294 | ethynyl | 2-methoxyphenyl |
| I-1295 | ethynyl | 3-chlorophenyl |
| I-1296 | ethynyl | 3-fluorophenyl |
| I-1297 | ethynyl | 3-trifluoromethyl- |
| | | phenyl |
| I-1298 | ethynyl | 3-methoxyphenyl |
| I-1299 | ethynyl | 3-methylphenyl |
| I-1300 | ethynyl | 4-chlorophenyl |
| I-1301 | ethynyl | 4-fluorophenyl |
| I-1302 | ethynyl | 4-methylphenyl |
| I-1303 | ethynyl | 4-methoxyphenyl |
| I-1304 | ethynyl | 4-trifluoromethyl- |
| | | phenyl |
| I-1305 | ethynyl | 9H-fluoren-9-yl |
| I-1306 | ethynyl- | ethyl |
| | Si(CH₃)₃ | |
| I-1307 | ethynyl- | n-propyl |
| | Si(CH₃)₃ | |
| I-1308 | ethynyl- | 1-methyl-ethyl |
| | Si(CH₃)₃ | |
| I-1309 | ethynyl- | n-butyl |
| | Si(CH₃)₃ | |
| I-1310 | ethynyl- | 2-methyl-propyl |
| | Si(CH₃)₃ | |
| I-1311 | ethynyl- | 3-methyl-butyl |
| | Si(CH₃)₃ | |
| I-1312 | ethynyl- | 2,2-dimethyl-propyl |
| | Si(CH₃)₃ | |
| I-1313 | ethynyl- | 3,3-dimethyl-butyl |
| | Si(CH₃)₃ | |
| I-1314 | ethynyl- | 4,4-dimethyl-pentyl |
| | Si(CH₃)₃ | |
| I-1315 | ethynyl- | 2-ethyl-hexyl |
| | Si(CH₃)₃ | |
| I-1316 | ethynyl- | 3,5,5-trimethyl-hexyl |
| | Si(CH₃)₃ | |
| I-1317 | ethynyl- | 2-chloro-ethyl |
| | Si(CH₃)₃ | |
| I-1318 | ethynyl- | 2-tert-butoxy-propyl |
| | Si(CH₃)₃ | |
| I-1319 | ethynyl- | 2,3-diacetoxy-propyl |
| | Si(CH₃)₃ | |
| I-1320 | ethynyl- | 3-methoxycarbonyl- |
| | Si(CH₃)₃ | propyl |
| I-1321 | ethynyl- | 3-methylsulfanyl- |
| | Si(CH₃)₃ | propyl |
| I-1322 | ethynyl- | 2,3-dihydroxy- |
| | Si(CH₃)₃ | propyl |
| I-1323 | ethynyl- | 2-hydroxy-butyl |
| | Si(CH₃)₃ | |
| I-1324 | ethynyl- | 2-acetoxy-butyl |
| | Si(CH₃)₃ | |
| I-1325 | ethynyl- | 2-benzyloxy-butyl |
| | Si(CH₃)₃ | |
| I-1326 | ethynyl- | 2-dimethylamino-2- |
| | Si(CH₃)₃ | methyl-propyl |
| I-1327 | ethynyl- | 1-phenylethynyl- |
| | Si(CH₃)₃ | butyl |
| I-1328 | ethynyl- | allyl |
| | Si(CH₃)₃ | |
| I-1329 | ethynyl- | 2,6-dimethyl-hept-5- |
| | Si(CH₃)₃ | enyl |
| I-1330 | ethynyl- | (Z)-dec-4-enyl |
| | Si(CH₃)₃ | |
| I-1331 | ethynyl- | 2-(2,6,6-trimethyl- |
| | Si(CH₃)₃ | cyclohex-1-enyl) |
| | | ethyl |
| I-1332 | ethynyl- | 1-naphtalene |
| | Si(CH₃)₃ | methyl |
| I-1333 | ethynyl- | 2-naphtalene- |
| | Si(CH₃)₃ | methyl |
| I-1334 | ethynyl- | phenethyl |
| | Si(CH₃)₃ | |
| I-1335 | ethynyl- | 2-phenyl-propyl |
| | Si(CH₃)₃ | |
| I-1336 | ethynyl- | 3-phenyl-propyl |
| | Si(CH₃)₃ | |
| I-1337 | ethynyl- | 4-benzo[1,3]dioxol- |
| | Si(CH₃)₃ | 5-yl-butyl |
| I-1338 | ethynyl- | cyclopropyl-methyl |
| | Si(CH₃)₃ | |
| I-1339 | ethynyl- | 1-(4-chloro-phenyl)- |
| | Si(CH₃)₃ | 1H-pyrrol-3-ylmethyl |
| I-1340 | ethynyl- | furan-2-ylmethyl |
| | Si(CH₃)₃ | |
| I-1341 | ethynyl- | furan-3-ylmethyl |
| | Si(CH₃)₃ | |
| I-1342 | ethynyl- | 3- |
| | Si(CH₃)₃ | dimethylaminomethyl- |
| | | tetrahydro-furan- |
| | | 3-ylmethyl |
| I-1343 | ethynyl- | 2,5-dihydro- |
| | Si(CH₃)₃ | thiophen-3-ylmethyl |
| I-1344 | ethynyl- | 1-tert-butyl-1H- |
| | Si(CH₃)₃ | pyrazol-4-ylmethyl |
| I-1345 | ethynyl- | 5-methyl-1H- |
| | Si(CH₃)₃ | imidazol-4-ylmethyl |
| I-1346 | ethynyl- | 2-chloro-thiazol-5- |
| | Si(CH₃)₃ | ylmethyl |
| I-1347 | ethynyl- | 3-isobutyl-isoxazol- |
| | Si(CH₃)₃ | 5-ylmethyl |
| I-1348 | ethynyl- | 1-methyl-1H-pyrrol- |
| | Si(CH₃)₃ | 2-ylmethyl |
| I-1349 | ethynyl- | tetrahydro-pyran-3- |
| | Si(CH₃)₃ | ylmethyl |
| I-1350 | ethynyl- | tetrahydro-pyran-4- |
| | Si(CH₃)₃ | ylmethyl |
| I-1351 | ethynyl- | tetrahydro- |
| | Si(CH₃)₃ | thiopyran-3-ylmethyl |
| I-1352 | ethynyl- | thiophen-2-ylmethyl |
| | Si(CH₃)₃ | |
| I-1353 | ethynyl- | pyridin-2-ylmethyl |
| | Si(CH₃)₃ | |
| I-1354 | ethynyl- | pyridin-3-ylmethyl |
| | Si(CH₃)₃ | |
| I-1355 | ethynyl- | pyridin-4-ylmethyl |
| | Si(CH₃)₃ | |
| I-1356 | ethynyl- | 6-chloro-pyridin-3- |
| | Si(CH₃)₃ | ylmethyl |
| I-1357 | ethynyl- | 1H-indol-3-ylmethyl |
| | Si(CH₃)₃ | |
| I-1358 | ethynyl- | 3-chloro-1H-indol-2- |
| | Si(CH₃)₃ | ylmethyl |
| I-1359 | ethynyl- | 2-(tetrahydro-pyran- |
| | Si(CH₃)₃ | 4-yl)-ethyl |
| I-1360 | ethynyl- | 2,2-dimethyl-3- |
| | Si(CH₃)₃ | pyridin-3-yl-propyl |
| I-1361 | ethynyl- | benzyl |
| | Si(CH₃)₃ | |
| I-1362 | ethynyl- | 2-amino-benzyl |
| | Si(CH₃)₃ | |
| I-1363 | ethynyl- | 2-bromo-benzyl |
| | Si(CH₃)₃ | |
| I-1364 | ethynyl- | 2-fluoro-benzyl |
| | Si(CH₃)₃ | |
| I-1365 | ethynyl- | 2-chloro-benzyl |
| | Si(CH₃)₃ | |
| I-1366 | ethynyl- | 2-cyano-benzyl |
| | Si(CH₃)₃ | |
| I-1367 | ethynyl- | 2-hydroxy-benzyl |
| | Si(CH₃)₃ | |
| I-1368 | ethynyl- | 2-methyl-benzyl |
| | Si(CH₃)₃ | |
| I-1369 | ethynyl- | 2-methoxy-benzyl |
| | Si(CH₃)₃ | |
| I-1370 | ethynyl- | 2-trifluoromethyl- |
| | Si(CH₃)₃ | benzyl |
| I-1371 | ethynyl- | 2-trifluoromethoxy- |
| | Si(CH₃)₃ | benzyl |
| I-1372 | ethynyl- | 2-diphenyl- |
| | Si(CH₃)₃ | phosphino-benzyl |
| I-1373 | ethynyl- | 2-benzyloxy-benzyl |
| | Si(CH₃)₃ | |
| I-1374 | ethynyl- | 2,4-dichloro-benzyl |
| | Si(CH₃)₃ | |
| I-1375 | ethynyl- | 2,5-dichloro-benzyl |
| | Si(CH₃)₃ | |
| I-1376 | ethynyl- | 2,6-dichloro-benzyl |
| | Si(CH₃)₃ | |
| I-1377 | ethynyl- | 2-fluoro, 4-bromo- |
| | Si(CH₃)₃ | benzyl |
| I-1378 | ethynyl- | 2-methyl,4-tert- |
| | Si(CH₃)₃ | butyl-benzyl |
| I-1379 | ethynyl- | 2-chloro, |
| | Si(CH₃)₃ | 6-methanesulfanyl- |
| | | benzyl |
| I-1380 | ethynyl- | 2-chloro, |
| | Si(CH₃)₃ | 6-methanesulfinyl- |
| | | benzyl |
| I-1381 | ethynyl- | 2-methanesulfanyl, |
| | Si(CH₃)₃ | 5-nitro-benzyl |
| I-1382 | ethynyl- | 2-methanesulfinyl, |
| | Si(CH₃)₃ | 5-nitro-benzyl |
| I-1383 | ethynyl- | 3-amino-benzyl |
| | Si(CH₃)₃ | |
| I-1384 | ethynyl- | 3-bromo-benzyl |
| | Si(CH₃)₃ | |
| I-1385 | ethynyl- | 3-chloro-benzyl |
| | Si(CH₃)₃ | |
| I-1386 | ethynyl- | 3-fluoro-benzyl |
| | Si(CH₃)₃ | |
| I-1387 | ethynyl- | 3-cyano-benzyl |
| | Si(CH₃)₃ | |
| I-1388 | ethynyl- | 3-hydroxy-benzyl |
| | Si(CH₃)₃ | |
| I-1389 | ethynyl- | 3-methoxy-benzyl |
| | Si(CH₃)₃ | |
| I-1390 | ethynyl- | 3-methyl-benzyl |
| | Si(CH₃)₃ | |
| I-1391 | ethynyl- | 3-nitro-benzyl |
| | Si(CH₃)₃ | |
| I-1392 | ethynyl- | 3-benzyloxy-benzyl |
| | Si(CH₃)₃ | |
| I-1393 | ethynyl- | 3-trifluoromethyl- |
| | Si(CH₃)₃ | benzyl |
| I-1394 | ethynyl- | 3-trifluoromethoxy- |
| | Si(CH₃)₃ | benzyl |
| I-1395 | ethynyl- | 3,5-dichloro-benzyl |
| | Si(CH₃)₃ | |
| I-1396 | ethynyl- | 3,5-di-tert-butyl-4- |
| | Si(CH₃)₃ | hydroxy-benzyl |
| I-1397 | ethynyl- | 3-bromo, 4-fluoro- |
| | Si(CH₃)₃ | benzyl |
| I-1398 | ethynyl- | 3-bromo, |
| | Si(CH₃)₃ | 6-fluoro-benzyl |
| I-1399 | ethynyl- | 3-fluoro, |
| | Si(CH₃)₃ | 6-methanesulfinyl- |
| | | benzyl |
| I-1400 | ethynyl- | 3-fluoro, 6- |
| | Si(CH₃)₃ | methanesulfanyl- |
| | | benzyl |
| I-1401 | ethynyl- | 3-chloro, |
| | Si(CH₃)₃ | 6-methanesulfinyl- |
| | | benzyl |
| I-1402 | ethynyl- | 3-chloro, 6- |
| | Si(CH₃)₃ | methanesulfanyl- |
| | | benzyl |
| I-1403 | ethynyl- | 4-amino-benzyl |
| | Si(CH₃)₃ | |
| I-1404 | ethynyl- | 4-benzyloxy-benzyl |
| | Si(CH₃)₃ | |
| I-1405 | ethynyl- | 4-bromo-benzyl |
| | Si(CH₃)₃ | |
| I-1406 | ethynyl- | 4-chloro-benzyl |
| | Si(CH₃)₃ | |
| I-1407 | ethynyl- | 4-fluoro-benzyl |
| | Si(CH₃)₃ | |
| I-1408 | ethynyl- | 4-cyano-benzyl |
| | Si(CH₃)₃ | |
| I-1409 | ethynyl- | 4-acetylamino- |
| | Si(CH₃)₃ | benzyl |
| I-1410 | ethynyl- | 4-dimethylamino- |
| | Si(CH₃)₃ | benzyl |
| I-1411 | ethynyl- | 4-hydroxy-benzyl |
| | Si(CH₃)₃ | |
| I-1412 | ethynyl- | 4-methyl-benzyl |
| | Si(CH₃)₃ | |
| I-1413 | ethynyl- | 4-methoxy-benzyl |
| | Si(CH₃)₃ | |
| I-1414 | ethynyl- | 4-methoxycarbonyl- |
| | Si(CH₃)₃ | benzyl |
| I-1415 | ethynyl- | 4-methanesulfanyl- |
| | Si(CH₃)₃ | benzyl |
| I-1416 | ethynyl- | 4-methanesulfinyl- |
| | Si(CH₃)₃ | benzyl |
| I-1417 | ethynyl- | 4-N,N-dimethyl- |
| | Si(CH₃)₃ | amino-benzyl |
| I-1418 | ethynyl- | 4-nitro-benzyl |
| | Si(CH₃)₃ | |
| I-1419 | ethynyl- | 4-tert-butyl-benzyl |
| | Si(CH₃)₃ | |
| I-1420 | ethynyl- | 4-trifluoromethyl- |
| | Si(CH₃)₃ | benzyl |
| I-1421 | ethynyl- | 4-trifluoromethoxy- |
| | Si(CH₃)₃ | benzyl |
| I-1422 | ethynyl- | 2,4,6-trimethoxy- |
| | Si(CH₃)₃ | benzyl |
| I-1423 | ethynyl- | 2,3,4,5,6- |
| | Si(CH₃)₃ | pentamethyl-benzyl |
| I-1424 | ethynyl- | 2-(4-fluoro-phenyl)- |
| | Si(CH₃)₃ | 4-methyl-pentyl |
| I-1425 | ethynyl- | 2-[1-phenyl-meth- |
| | Si(CH₃)₃ | (E)-ylidene]-heptyl |
| I-1426 | ethynyl- | 1-phenyl-prop-2- |
| | Si(CH₃)₃ | ynyl |
| I-1427 | ethynyl- | 1-phenyl-3- |
| | Si(CH₃)₃ | trimethylsilanyl- |
| | | prop-2-ynyl |
| I-1428 | ethynyl- | 4-ethoxycarbonyl-1- |
| | Si(CH₃)₃ | phenyl-but-2-ynyl |
| I-1429 | ethynyl- | 4-methoxy-1- |
| | Si(CH₃)₃ | phenyl-but-2-ynyl |
| I-1430 | ethynyl- | 4,4-dimethyl-1- |
| | Si(CH₃)₃ | phenyl-pent-2-ynyl |
| I-1431 | ethynyl- | 1-phenyl-hex-2-ynyl |
| | Si(CH₃)₃ | |
| I-1432 | ethynyl- | 1-(4-chloro-phenyl)- |
| | Si(CH₃)₃ | hex-2-ynyl |
| I-1433 | ethynyl- | 1-(4-methoxy- |
| | Si(CH₃)₃ | phenyl)-hex-2-ynyl |
| I-1434 | ethynyl- | phenyl |
| | Si(CH₃)₃ | |
| I-1435 | ethynyl- | 2-chlorophenyl |
| | Si(CH₃)₃ | |
| I-1436 | ethynyl- | 2-fluorophenyl |
| | Si(CH₃)₃ | |
| I-1437 | ethynyl- | 2-trifluoromethyl- |
| | Si(CH₃)₃ | phenyl |
| I-1438 | ethynyl- | 2-methylphenyl |
| | Si(CH₃)₃ | |
| I-1439 | ethynyl- | 2-methoxyphenyl |
| | Si(CH₃)₃ | |
| I-1440 | ethynyl- | 3-chlorophenyl |
| | Si(CH₃)₃ | |
| I-1441 | ethynyl- | 3-fluorophenyl |
| | Si(CH₃)₃ | |
| I-1442 | ethynyl- | 3-trifluoromethyl- |
| | Si(CH₃)₃ | phenyl |
| I-1443 | ethynyl- | 3-methoxyphenyl |
| | Si(CH₃)₃ | |
| I-1444 | ethynyl- | 3-methylphenyl |
| | Si(CH₃)₃ | |
| I-1445 | ethynyl- | 4-chlorophenyl |
| | Si(CH₃)₃ | |
| I-1446 | ethynyl- | 4-fluorophenyl |
| | Si(CH₃)₃ | |
| I-1447 | ethynyl- | 4-methylphenyl |
| | Si(CH₃)₃ | |
| I-1448 | ethynyl- | 4-methoxyphenyl |
| | Si(CH₃)₃ | |
| I-1449 | ethynyl- | 4-trifluoromethyl- |
| | Si(CH₃)₃ | phenyl |
| I-1450 | ethynyl- | 9H-fluoren-9-yl |
| | Si(CH₃)₃ | |
| I-1451 | 1-propynyl | ethyl |
| I-1452 | 1-propynyl | n-propyl |
| I-1453 | 1-propynyl | 1-methyl-ethyl |
| I-1454 | 1-propynyl | n-butyl |
| I-1455 | 1-propynyl | 2-methyl-propyl |
| I-1456 | 1-propynyl | 3-methyl-butyl |
| I-1457 | 1-propynyl | 2,2-dimethyl-propyl |
| I-1458 | 1-propynyl | 3,3-dimethyl-butyl |
| I-1459 | 1-propynyl | 4,4-dimethyl-pentyl |
| I-1460 | 1-propynyl | 2-ethyl-hexyl |
| I-1461 | 1-propynyl | 3,5,5-trimethyl-hexyl |
| I-1462 | 1-propynyl | 2-chloro-ethyl |
| I-1463 | 1-propynyl | 2-tert-butoxy-propyl |
| I-1464 | 1-propynyl | 2,3-diacetoxy-propyl |
| I-1465 | 1-propynyl | 3-methoxycarbonyl- |
| | | propyl |
| I-1466 | 1-propynyl | 3-methylsulfanyl- |
| | | propyl |
| I-1467 | 1-propynyl | 2,3-dihydroxy- |
| | | propyl |
| I-1468 | 1-propynyl | 2-hydroxy-butyl |
| I-1469 | 1-propynyl | 2-acetoxy-butyl |
| I-1470 | 1-propynyl | 2-benzyloxy-butyl |
| I-1471 | 1-propynyl | 2-dimethylamino-2- |
| | | methyl-propyl |
| I-1472 | 1-propynyl | 1-phenylethynyl- |
| | | butyl |
| I-1473 | 1-propynyl | allyl |
| I-1474 | 1-propynyl | 2,6-dimethyl-hept-5- |
| | | enyl |
| I-1475 | 1-propynyl | (Z)-dec-4-enyl |
| I-1476 | 1-propynyl | 2-(2,6,6-trimethyl- |
| | | cyclohex-1-enyl)- |
| | | ethyl |
| I-1477 | 1-propynyl | 1-naphtalene- |
| | | methyl |
| I-1478 | 1-propynyl | 2-naphtalene- |
| | | methyl |
| I-1479 | 1-propynyl | phenethyl |
| I-1480 | 1-propynyl | 2-phenyl-propyl |
| I-1481 | 1-propynyl | 3-phenyl-propyl |
| I-1482 | 1-propynyl | 4-benzo[1,3]dioxol- |
| | | 5-yl-butyl |
| I-1483 | 1-propynyl | cyclopropyl-methyl |
| I-1484 | 1-propynyl | 1-(4-chloro-phenyl)- |
| | | 1H-pyrrol-3-ylmethyl |
| I-1485 | 1-propynyl | furan-2-ylmethyl |
| I-1486 | 1-propynyl | furan-3-ylmethyl |
| I-1487 | 1-propynyl | 3- |
| | | dimethylaminomethyl- |
| | | tetrahydro-furan- |
| | | 3-ylmethyl |
| I-1488 | 1-propynyl | 2,5-dihydro- |
| | | thiophen-3-ylmethyl |
| I-1489 | 1-propynyl | 1-tert-butyl-1H- |
| | | pyrazol-4-ylmethyl |
| I-1490 | 1-propynyl | 5-methyl-1H- |
| | | imidazol-4-ylmethyl |
| I-1491 | 1-propynyl | 2-chloro-thiazol-5- |
| | | ylmethyl |
| I-1492 | 1-propynyl | 3-isobutyl-isoxazol- |
| | | 5-ylmethyl |
| I-1493 | 1-propynyl | 1-methyl-1H-pyrrol- |
| | | 2-ylmethyl |
| I-1494 | 1-propynyl | tetrahydro-pyran-3- |
| | | ylmethyl |
| I-1495 | 1-propynyl | tetrahydro-pyran-4- |
| | | ylmethyl |
| I-1496 | 1-propynyl | tetrahydro- |
| | | thiopyran-3-ylmethyl |
| I-1497 | 1-propynyl | thiophen-2-ylmethyl |
| I-1498 | 1-propynyl | pyridin-2-ylmethyl |
| I-1499 | 1-propynyl | pyridin-3-ylmethyl |
| I-1500 | 1-propynyl | pyridin-4-ylmethyl |
| I-1501 | 1-propynyl | 6-chloro-pyridin-3- |
| | | ylmethyl |
| I-1502 | 1-propynyl | 1H-indol-3-ylmethyl |
| I-1503 | 1-propynyl | 3-chloro-1H-indol-2- |
| | | ylmethyl |
| I-1504 | 1-propynyl | 2-(tetrahydro-pyran- |
| | | 4-yl)-ethyl |
| I-1505 | 1-propynyl | 2,2-dimethyl-3- |
| | | pyridin-3-yl-propyl |
| I-1506 | 1-propynyl | benzyl |
| I-1507 | 1-propynyl | 2-amino-benzyl |
| I-1508 | 1-propynyl | 2-bromo-benzyl |
| I-1509 | 1-propynyl | 2-fluoro-benzyl |
| I-1510 | 1-propynyl | 2-chloro-benzyl |
| I-1511 | 1-propynyl | 2-cyano-benzyl |
| I-1512 | 1-propynyl | 2-hydroxy-benzyl |
| I-1513 | 1-propynyl | 2-methyl-benzyl |
| I-1514 | 1-propynyl | 2-methoxy-benzyl |
| I-1515 | 1-propynyl | 2-trifluoromethyl- |
| | | benzyl |
| I-1516 | 1-propynyl | 2-trifluoromethoxy- |
| | | benzyl |
| I-1517 | 1-propynyl | 2-diphenyl- |
| | | phosphino-benzyl |
| I-1518 | 1-propynyl | 2-benzyloxy-benzyl |
| I-1519 | 1-propynyl | 2,4-dichloro-benzyl |
| I-1520 | 1-propynyl | 2,5-dichloro-benzyl |
| I-1521 | 1-propynyl | 2,6-dichloro-benzyl |
| I-1522 | 1-propynyl | 2-fluoro, 4-bromo- |
| | | benzyl |
| I-1523 | 1-propynyl | 2-methyl,4-tert- |
| | | butyl-benzyl |
| I-1524 | 1-propynyl | 2-chloro, |
| | | 6-methanesulfanyl- |
| | | benzyl |
| I-1525 | 1-propynyl | 2-chloro, |
| | | 6-methanesulfinyl- |
| | | benzyl |
| I-1526 | 1-propynyl | 2-methanesulfanyl, |
| | | 5-nitro-benzyl |
| I-1527 | 1-propynyl | 2-methanesulfinyl, |
| | | 5-nitro-benzyl |
| I-1528 | 1-propynyl | 3-amino-benzyl |
| I-1529 | 1-propynyl | 3-bromo-benzyl |
| I-1530 | 1-propynyl | 3-chloro-benzyl |
| I-1531 | 1-propynyl | 3-fluoro-benzyl |
| I-1532 | 1-propynyl | 3-cyano-benzyl |
| I-1533 | 1-propynyl | 3-hydroxy-benzyl |
| I-1534 | 1-propynyl | 3-methoxy-benzyl |
| I-1535 | 1-propynyl | 3-methyl-benzyl |
| I-1536 | 1-propynyl | 3-nitro-benzyl |
| I-1537 | 1-propynyl | 3-benzyloxy-benzyl |
| I-1538 | 1-propynyl | 3-trifluoromethyl- |
| | | benzyl |
| I-1539 | 1-propynyl | 3-trifluoromethoxy- |
| | | benzyl |
| I-1540 | 1-propynyl | 3,5-dichloro-benzyl |
| I-1541 | 1-propynyl | 3,5-di-tert-butyl-4- |
| | | hydroxy-benzyl |
| I-1542 | 1-propynyl | 3-bromo, 4-fluoro- |
| | | benzyl |
| I-1543 | 1-propynyl | 3-bromo, |
| | | 6-fluoro-benzyl |
| I-1544 | 1-propynyl | 3-fluoro, |
| | | 6-methanesulfinyl- |
| | | benzyl |
| I-1545 | 1-propynyl | 3-fluoro, 6- |
| | | methanesulfanyl- |
| | | benzyl |
| I-1546 | 1-propynyl | 3-chloro, |
| | | 6-methanesulfanyl- |
| | | benzyl |
| I-1547 | 1-propynyl | 3-chloro, 6- |
| | | methanesulfanyl- |
| | | benzyl |
| I-1548 | 1-propynyl | 4-amino-benzyl |
| I-1549 | 1 propynyl | 4-benzyloxy-benzyl |
| I-1550 | 1-propynyl | 4-bromo-benzyl |
| I-1551 | 1-propynyl | 4-chloro-benzyl |
| I-1552 | 1-propynyl | 4-fluoro-benzyl |
| I-1553 | 1-propynyl | 4-cyano-benzyl |
| I-1554 | 1-propynyl | 4-acetylamino- |
| | | benzyl |
| I-1555 | 1-propynyl | 4-dimethylamino- |
| | | benzyl |
| I-1556 | 1-propynyl | 4-hydroxy-benzyl |
| I-1557 | 1-propynyl | 4-methyl-benzyl |
| I-1558 | 1-propynyl | 4-methoxy-benzyl |
| I-1559 | 1-propynyl | 4-methoxycarbonyl- |
| | | benzyl |
| I-1560 | 1-propynyl | 4-methanesulfanyl- |
| | | benzyl |
| I-1561 | 1-propynyl | 4-methanesulfinyl- |
| | | benzyl |
| I-1562 | 1-propynyl | 4-N,N-dimethyl- |
| | | amino-benzyl |
| I-1563 | 1-propynyl | 4-nitro-benzyl |
| I-1564 | 1-propynyl | 4-tert-butyl-benzyl |
| I-1565 | 1-propynyl | 4-trifluoromethyl- |
| | | benzyl |
| I-1566 | 1-propynyl | 4-trifluoromethoxy- |
| | | benzyl |
| I-1567 | 1-propynyl | 2,4,6-trimethoxy- |
| | | benzyl |
| I-1568 | 1-propynyl | 2,3,4,5,6- |
| | | pentamethyl-benzyl |
| I-1569 | 1-propynyl | 2-(4-fluoro-phenyl)- |
| | | 4-methyl-pentyl |
| I-1570 | 1-propynyl | 2-[1-phenyl-meth- |
| | | (E)-ylidene]-heptyl |
| I-1571 | 1-propynyl | 1-phenyl-prop-2- |
| | | ynyl |
| I-1572 | 1-propynyl | 1-phenyl-3- |
| | | trimethylsilanyl- |
| | | prop-2-ynyl |
| I-1573 | 1-propynyl | 4-ethoxycarbonyl-1- |
| | | phenyl-but-2-ynyl |
| I-1574 | 1-propynyl | 4-methoxy-1- |
| | | phenyl-but-2-ynyl |
| I-1575 | 1-propynyl | 4,4-dimethyl-1- |
| | | phenyl-pent-2-ynyl |
| I-1576 | 1-propynyl | 1-phenyl-hex-2-ynyl |
| I-1577 | 1-propynyl | 1-(4-chloro-phenyl)- |
| | | hex-2-ynyl |
| I-1578 | 1-propynyl | 1-(4-methoxy- |
| | | phenyl)-hex-2-ynyl |
| I-1579 | 1-propynyl | phenyl |
| I-1580 | 1-propynyl | 2-chlorophenyl |
| I-1581 | 1-propynyl | 2-fluorophenyl |
| I-1582 | 1-propynyl | 2-trifluoromethyl- |
| | | phenyl |
| I-1583 | 1-propynyl | 2-methylphenyl |
| I-1584 | 1-propynyl | 2-methoxyphenyl |
| I-1585 | 1-propynyl | 3-chlorophenyl |
| I-1586 | 1-propynyl | 3-fluorophenyl |
| I-1587 | 1-propynyl | 3-trifluoromethyl- |
| | | phenyl |
| I-1588 | 1-propynyl | 3-methoxyphenyl |
| I-1589 | 1-propynyl | 3-methylphenyl |
| I-1590 | 1-propynyl | 4-chlorophenyl |
| I-1591 | 1-propynyl | 4-fluorophenyl |
| I-1592 | 1-propynyl | 4-methylphenyl |
| I-1593 | 1-propynyl | 4-methoxyphenyl |
| I-1594 | 1-propynyl | 4-trifluoromethyl- |
| | | phenyl |
| I-1595 | 1-propynyl | 9H-fluoren-9-yl |
| I-1596 | CCCH₂OH | ethyl |
| I-1597 | CCCH₂OH | n-propyl |
| I-1598 | CCCH₂OH | 1-methyl-ethyl |
| I-1599 | CCCH₂OH | n-butyl |
| I-1600 | CCCH₂OH | 2-methyl-propyl |
| I-1601 | CCCH₂OH | 3-methyl-butyl |
| I-1602 | CCCH₂OH | 2,2-dimethyl-propyl |
| I-1603 | CCCH₂OH | 3,3-dimethyl-butyl |
| I-1604 | CCCH₂OH | 4,4-dimethyl-pentyl |
| I-1605 | CCCH₂OH | 2-ethyl-hexyl |
| I-1606 | CCCH₂OH | 3,5,5-trimethyl-hexyl |
| I-1607 | CCCH₂OH | 2-chloro-ethyl |
| I-1608 | CCCH₂OH | 2-tert-butoxy-propyl |
| I-1609 | CCCH₂OH | 2,3-diacetoxy-propyl |
| I-1610 | CCCH₂OH | 3-methoxycarbonyl- |
| | | propyl |
| I-1611 | CCCH₂OH | 3-methylsulfanyl- |
| | | propyl |
| I-1612 | CCCH₂OH | 2,3-dihydroxy- |
| | | propyl |
| I-1613 | CCCH₂OH | 2-hydroxy-butyl |
| I-1614 | CCCH₂OH | 2-acetoxy-butyl |
| I-1615 | CCCH₂OH | 2-benzyloxy-butyl |
| I-1616 | CCCH₂OH | 2-dimethylamino-2- |
| | | methyl-propyl |
| I-1617 | CCCH₂OH | 1-phenylethynyl- |
| | | butyl |
| I-1618 | CCCH₂OH | allyl |
| I-1619 | CCCH₂OH | 2,6-dimethyl-hept-5- |
| | | enyl |
| I-1620 | CCCH₂OH | (Z)-dec-4-enyl |
| I-1621 | CCCH₂OH | 2-(2,6,6-trimethyl- |
| | | cyclohex-1-enyl)- |
| | | ethyl |
| I-1622 | CCCH₂OH | 1-naphtalene- |
| | | methyl |
| I-1623 | CCCH₂OH | 2-naphtalene- |
| | | methyl |
| I-1624 | CCCH₂OH | phenethyl |
| I-1625 | CCCH₂OH | 2-phenyl-propyl |
| I-1626 | CCCH₂OH | 3-phenyl-propyl |
| I-1627 | CCCH₂OH | 4-benzo[1,3]dioxol- |
| | | 5-yl-butyl |
| I-1628 | CCCH₂OH | cyclopropyl-methyl |
| I-1629 | CCCH₂OH | 1-(4-chloro-phenyl)- |
| | | 1H-pyrrol-3-ylmethyl |
| I-1630 | CCCH₂OH | furan-2-ylmethyl |
| I-1631 | CCCH₂OH | furan-3-ylmethyl |
| I-1632 | CCCH₂OH | 3- |
| | | dimethylaminomethyl- |
| | | tetrahydro-furan- |
| | | 3-ylmethyl |
| I-1633 | CCCH₂OH | 2,5-dihydro- |
| | | thiophen-3-ylmethyl |
| I-1634 | CCCH₂OH | 1-tert-butyl-1H- |
| | | pyrazol-4-ylmethyl |
| I-1635 | CCCH₂OH | 5-methyl-1H- |
| | | imidazol-4-ylmethyl |
| I-1636 | CCCH₂OH | 2-chloro-thiazol-5- |
| | | ylmethyl |
| I-1637 | CCCH₂OH | 3-isobutyl-isoxazol- |
| | | 5-ylmethyl |
| I-1638 | CCCH₂OH | 1-methyl-1H-pyrrol- |
| | | 2-ylmethyl |
| I-1639 | CCCH₂OH | tetrahydro-pyran-3- |
| | | ylmethyl |
| I-1640 | CCCH₂OH | tetrahydro-pyran-4- |
| | | ylmethyl |
| I-1641 | CCCH₂OH | tetrahydro- |
| | | thiopyran-3-ylmethyl |
| I-1642 | CCCH₂OH | thiophen-2-ylmethyl |
| I-1643 | CCCH₂OH | pyridin-2-ylmethyl |
| I-1644 | CCCH₂OH | pyridin-3-ylmethyl |
| I-1645 | CCCH₂OH | pyridin-4-ylmethyl |
| I-1646 | CCCH₂OH | 6-chloro-pyridin-3- |
| | | ylmethyl |
| I-1647 | CCCH₂OH | 1H-indol-3-ylmethyl |
| I-1648 | CCCH₂OH | 3-chloro-1H-indol-2- |
| | | ylmethyl |
| I-1649 | CCCH₂OH | 2-(tetrahydro-pyran- |
| | | 4-yl)-ethyl |
| I-1650 | CCCH₂OH | 2,2-dimethyl-3- |
| | | pyridin-3-yl-propyl |
| I-1651 | CCCH₂OH | benzyl |
| I-1652 | CCCH₂OH | 2-amino-benzyl |
| I-1653 | CCCH₂OH | 2-bromo-benzyl |
| I-1654 | CCCH₂OH | 2-fluoro-benzyl |
| I-1655 | CCCH₂OH | 2-chloro-benzyl |
| I-1656 | CCCH₂OH | 2-cyano-benzyl |
| I-1657 | CCCH₂OH | 2-hydroxy-benzyl |
| I-1658 | CCCH₂OH | 2-methyl-benzyl |
| I-1659 | CCCH₂OH | 2-methoxy-benzyl |
| I-1660 | CCCH₂OH | 2-trifluoromethyl- |
| | | benzyl |
| I-1661 | CCCH₂OH | 2-trifluoromethoxy- |
| | | benzyl |
| I-1662 | CCCH₂OH | 2-diphenyl- |
| | | phosphino-benzyl |
| I-1663 | CCCH₂OH | 2-benzyloxy-benzyl |
| I-1664 | CCCH₂OH | 2,4-dichloro-benzyl |
| I-1665 | CCCH₂OH | 2,5-dichloro-benzyl |
| I-1666 | CCCH₂OH | 2,6-dichloro-benzyl |
| I-1667 | CCCH₂OH | 2-fluoro, 4-bromo- |
| | | benzyl |
| I-1668 | CCCH₂OH | 2-methyl,4-tert- |
| | | butyl-benzyl |
| I-1669 | CCCH₂OH | 2-chloro, |
| | | 6-methanesulfanyl- |
| | | benzyl |
| I-1670 | CCCH₂OH | 2-chloro, |
| | | 6-methanesulfinyl- |
| | | benzyl |
| I-1671 | CCCH₂OH | 2-methanesulfanyl, |
| | | 5-nitro-benzyl |
| I-1672 | CCCH₂OH | 2-methanesulfinyl, |
| | | 5-nitro-benzyl |
| I-1673 | CCCH₂OH | 3-amino-benzyl |
| I-1674 | CCCH₂OH | 3-bromo-benzyl |
| I-1675 | CCCH₂OH | 3-chloro-benzyl |
| I-1676 | CCCH₂OH | 3-fluoro-benzyl |
| I-1677 | CCCH₂OH | 3-cyano-benzyl |
| I-1678 | CCCH₂OH | 3-hydroxy-benzyl |
| I-1679 | CCCH₂OH | 3-methoxy-benzyl |
| I-1680 | CCCH₂OH | 3-methyl-benzyl |
| I-1681 | CCCH₂OH | 3-nitro-benzyl |
| I-1682 | CCCH₂OH | 3-benzyloxy-benzyl |
| I-1683 | CCCH₂OH | 3-trifluoromethyl- |
| | | benzyl |
| I-1684 | CCCH₂OH | 3-trifluoromethoxy- |
| | | benzyl |
| I-1685 | CCCH₂OH | 3,5-dichloro-benzyl |
| I-1686 | CCCH₂OH | 3,5-di-tert-butyl-4- |
| | | hydroxy-benzyl |
| I-1687 | CCCH₂OH | 3-bromo, 4-fluoro- |
| | | benzyl |
| I-1688 | CCCH₂OH | 3-bromo, |
| | | 6-fluoro-benzyl |
| I-1689 | CCCH₂OH | 3-fluoro, |
| | | 6-methanesulfinyl- |
| | | benzyl |
| I-1690 | CCCH₂OH | 3-fluoro, 6- |
| | | methanesulfanyl- |
| | | benzyl |
| I-1691 | CCCH₂OH | 3-chloro, |
| | | 6-methanesulfinyl- |
| | | benzyl |
| I-1692 | CCCH₂OH | 3-chloro, 6- |
| | | methanesulfanyl- |
| | | benzyl |
| I-1693 | CCCH₂OH | 4-amino-benzyl |
| I-1694 | CCCH₂OH | 4-benzyloxy-benzyl |
| I-1695 | CCCH₂OH | 4-bromo-benzyl |
| I-1696 | CCCH₂OH | 4-chloro-benzyl |
| I-1697 | CCCH₂OH | 4-fluoro-benzyl |
| I-1698 | CCCH₂OH | 4-cyano-benzyl |
| I-1699 | CCCH₂OH | 4-acetylamino- |
| | | benzyl |
| I-1700 | CCCH₂OH | 4-dimethylamino- |
| | | benzyl |
| I-1701 | CCCH₂OH | 4-hydroxy-benzyl |
| I-1702 | CCCH₂OH | 4-methyl-benzyl |
| I-1703 | CCCH₂OH | 4-methoxy-benzyl |
| I-1704 | CCCH₂OH | 4-methoxycarbonyl- |
| | | benzyl |
| I-1705 | CCCH₂OH | 4-methanesulfanyl- |
| | | benzyl |
| I-1706 | CCCH₂OH | 4-methanesulfinyl- |
| | | benzyl |
| I-1707 | CCCH₂OH | 4-N,N-dimethyl- |
| | | amino-benzyl |
| I-1708 | CCCH₂OH | 4-nitro-benzyl |
| I-1709 | CCCH₂OH | 4-tert-butyl-benzyl |
| I-1710 | CCCH₂OH | 4-trifluoromethyl- |
| | | benzyl |
| I-1711 | CCCH₂OH | 4-trifluoromethoxy- |
| | | benzyl |
| I-1712 | CCCH₂OH | 2,4,6-trimethoxy- |
| | | benzyl |
| I-1713 | CCCH₂OH | 2,3,4,5,6- |
| | | pentamethyl-benzyl |
| I-1714 | CCCH₂OH | 2-(4-fluoro-phenyl)- |
| | | 4-methyl-pentyl |
| I-1715 | CCCH₂OH | 2-[1-phenyl-meth- |
| | | (E)-ylidene]-heptyl |
| I-1716 | CCCH₂OH | 1-phenyl-prop-2- |
| | | ynyl |
| I-1717 | CCCH₂OH | 1-phenyl-3- |
| | | trimethylsilanyl- |
| | | prop-2-ynyl |
| I-1718 | CCCH₂OH | 4-ethoxycarbonyl-1- |
| | | phenyl-but-2-ynyl |
| I-1719 | CCCH₂OH | 4-methoxy-1- |
| | | phenyl-but-2-ynyl |
| I-1720 | CCCH₂OH | 4,4-dimethyl-1- |
| | | phenyl-pent-2-ynyl |
| I-1721 | CCCH₂OH | 1-phenyl-hex-2-ynyl |
| I-1722 | CCCH₂OH | 1-(4-chloro-phenyl)- |
| | | hex-2-ynyl |
| I-1723 | CCCH₂OH | 1-(4-methoxy- |
| | | phenyl)-hex-2-ynyl |
| I-1724 | CCCH₂OH | phenyl |
| I-1725 | CCCH₂OH | 2-chlorophenyl |
| I-1726 | CCCH₂OH | 2-fluorophenyl |
| I-1727 | CCCH₂OH | 2-trifluoromethyl- |
| | | phenyl |
| I-1728 | CCCH₂OH | 2-methylphenyl |
| I-1729 | CCCH₂OH | 2-methoxyphenyl |
| I-1730 | CCCH₂OH | 3-chlorophenyl |
| I-1731 | CCCH₂OH | 3-fluorophenyl |
| I-1732 | CCCH₂OH | 3-trifluoromethyl- |
| | | phenyl |
| I-1733 | CCCH₂OH | 3-methoxyphenyl |
| I-1734 | CCCH₂OH | 3-methylphenyl |
| I-1735 | CCCH₂OH | 4-chlorophenyl |
| I-1736 | CCCH₂OH | 4-fluorophenyl |
| I-1737 | CCCH₂OH | 4-methylphenyl |
| I-1738 | CCCH₂OH | 4-methoxyphenyl |
| I-1739 | CCCH₂OH | 4-trifluoromethyl- |
| | | phenyl |
| I-1740 | CCCH₂OH | 9H-fluoren-9-yl |

The compounds of the general formula I can be synthesized by techniques generally known in the art. A synthetic route to compounds of the formula I is shown in schemes 1, 2 and 3 below.

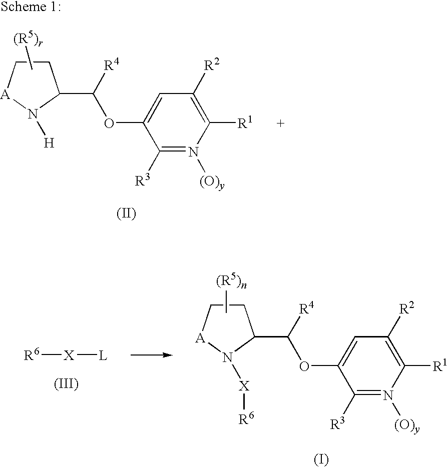

In scheme 1, A, X, n, y, R¹, R², R³, R⁴, R⁵ and R⁶ are as defined above. L is a leaving group which can be replaced by a nucleophile, examples including halogen such as chlorine, bromine, iodine, in particular bromine, alkylsulfonate such as mesylate and arylsulfonate such as phenylsulfonate and tolylsulfonate. According to the method of scheme 1, the amino compound of formula II is reacted with a compound of the formula III in sense of a nucleophilic substitution. The nucleophilic substitution can be accomplished by standard methods of nucleophilic substitution. For details, see R. B. Moffett, Org. Synth. IV, 466 (1963).

Preferably, the nucleophilic substitution is performed in the presence of a base. Suitable bases are organic bases, in particular tertiary amines like triethylamine, diisopropylamine, N-methyl morpholine, N-methylpiperidine, N-ethyl morpholine, N-ethylpiperidine or pyridine compounds like pyridine, picoline or lutidine. The base is used in a stoichiometry of 0.8 to 10 molar equivalents of compounds II and can also be used as a solvent.

Usually, the reaction is performed in a solvent such as aromatic hydrocarbons like benzene or toluene, halogenated aromatic hydrocarbons like chlorobenzene, halogenated hydrocarbons like dichloromethane, trichloromethane, dichloroethane, ethers such as tetrahydrofuran, ethyleneglycoldimethylether, methyl tertbutylether, amides like dimethylformamide or N-methylpyrrolidone, nitriles such as acetonitrile or propionitrile, and the mixtures thereof, with acetonitrile being preferred.

The nucleophilic substitution is usually run in a temperature range from 0 to 200° C., preferably from 45° C. to 120° C., in particular 75 to 90° C.

The reaction can be run under normal conditions or under microwave conditions.

The halogenated compounds of the formula III are commercially available or can be synthesized according to known procedures, e.g. by treatment of the corresponding alcohol with hydrogen halide, thionyl chloride or P(Hal)₃ with Hal being halogen [for example by bromation of the alcohol: Houben Weyl, 4. Ed., Vol 5/4, p 162-3] or by addition of hydrogen halides or halogens to the double bond of the corresponding alkenes [e.g., for the chlorination, see Houben Weyl 4. Ed., Vol. 5/3, p 529-542 and p 813-822]. Compounds of the formula II are known from the prior art cited in the introductory part of the present application.

Compounds of the formula I can also be prepared by reductive amination as outlined in scheme 2.

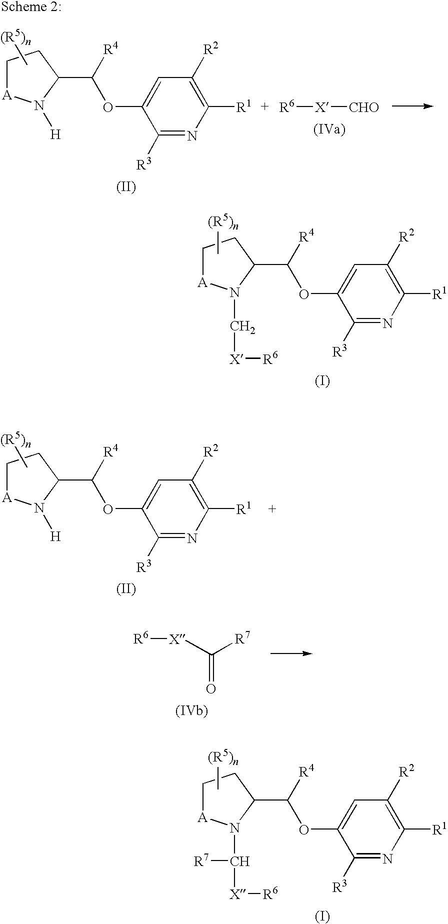

In scheme 2, A, n, R¹, R², R³, R⁴, R⁵, and R⁶ are as defined above. X′ is a chemical bond, CH₂ or CH₂CH₂, it being possible for CH₂ and CH₂CH₂ to carry 1 or 2 substituents R⁷ with R⁷ being as defined above. X″ is a chemical bond, CH₂ or CH₂CH₂, it being possible for CH₂ and CH₂CH₂ to carry 0, or 1 substituent R⁷ with R⁷ being as defined above.

According to the method of scheme 2, an aldehyde compound of the formula IVa or a ketone compound of formula IVb, respectively, is reacted with an amine compound of the formula II in the presence or a reducing agent to afford compound I. For details, see e.g. by J. K. Whitesell and by Robert and Marygail Hutchins in Comprehensive Organic Chemistry, B. Trost, I. Fleming, Eds. Pergamon Press, New York, 1991, resp. Vol. 6, p 705-726 and Vol 8, p 25-78.

The reaction of II with IVa or IVb, respectively, includes an addition step and reduction step. Both steps can be performed as separate reaction or as a one pot reaction. Suitable reduction agents are metal hydrides or boranes, with boranes being preferred. Examples of boranes include sodium cyanoborohydride or sodium triacetoxyborane. The borane can be a pure borane or a borane in a polymer supported form in order to facilitate the work-up and purification of the product. The reducing agent is usually used in a stoichiometry of 0.2 to 3 molar equivalents of compound II, preferably from 0.9 to 2.

The aldehyde compound IVa is used in the range of 1 to 3, preferably 0.9 to 1.2 molar equivalents of compound II. The ketone compound IVb is used in the range of 1 to 3, preferably 1.0 to 1.2 molar equivalents of compound II.

Usually, the reaction is performed in an inert organic solvent such as aromatic hydrocarbons like benzene or toluene, halogenated aromatic hydrocarbons like chlorobenzene, halogenated hydrocarbons like dichloromethane, trichloromethane, dichloroethane, ethers such as tetrahydrofuran, dioxane, ethyleneglycoldimethylether, methyl tert-butylether, amides like dimethylformamide or N-methylpyrrolidone, nitriles such as acetonitrile or propionitrile, and the mixtures thereof.

The aldehyde compounds IVa and the ketone compounds IVb are commercially available, or can be synthesized from the corresponding alcohol by oxidation. For details, see e.g. “Oxidation of alcohols to aldehydes and ketones” G. Tojo, M. Fernandez, Ed. Springer New York, 2006; Series: “Basic reactions in organic synthesis” or by ozonolyse of an alkene, see e.g. “Ozonization in Organic Synthesis”, P.S. Bailey, Vol 1, Academic Press, New York, 1978.

The aldehyde compound IVa can also be prepared from the corresponding acid or acid derivatives like acid-chloride or ester by reduction, see e.g. “Reductions in Organic Chemistry”, M. Hudlicky, 2^{nd} Ed., Ed. American Chemical Society, Chap. 14, 15, 16.

Compounds of the formula I with X being a bond, and R⁶ being an optionally substituted phenyl radical can be prepared e.g. as outlined in scheme 3.

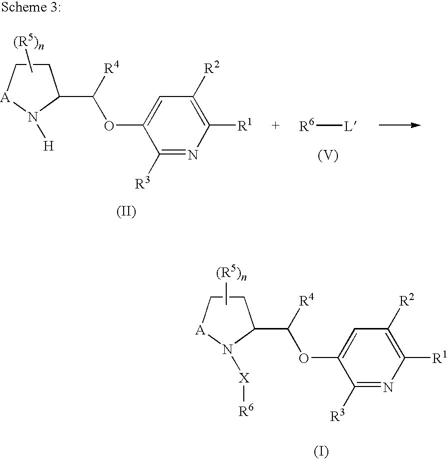

In scheme 3, A, n, R¹, R², R³, R⁴, and R⁵ are as defined above. L′ is halogen such as chlorine, bromine or iodine, triflate, nonaflate, alkoxide, such as C₁-C₆-alkoxide, tosylate, with bromine or iodine being preferred. According to the method of scheme 3, an amine compound of the formula II is reacted with a compound of the formula V by the Buchwald-Hartwig cross coupling reaction. For details, see e.g. [M. C. Harris, S. L. Buchwald, J. Org. Chem. 2000, 65, 5327-5333; B. Schlummer, U. Scholz Speciality Chemicals Magazine, May 2005, 22-24; Chimica Oggi, March/April 2005, 18-20.

The Buchwald-Hartwig cross coupling reaction is usually performed in the presence of a base. Suitable bases are bases such as carbonates, hydrogen carbonates, phosphates, hydrogen phosphates and dihydrogen phosphates of alkali metals or earth alkali metals such as NaHCO₃, KHCO₃, Na₂CO₃, K₂CO₃, Cs₂CO₃, NaH₂PO₄, Na₂HPO₄, KH₂PO₄, K₂HPO₄, K₃PO₄, sodium bis(trimethylsilyl)amide or alkali metal alkoxides such as sodium tert-butoxide or potassium tert-butoxide.

The reaction of compound II with compound V is usually performed in the presence of a transition metal catalyst, in particular a palladium(II) compound or palladium(0) compound as catalyst. The reaction is preferably performed in the presence of a phosphor containing ligand, in particular a mono- or bidentate phosphine ligand, which coordinates the palladium atoms of the catalyst. Preferred ligands on the palladium are bulky phosphines that can be monodentate or bidentate, examples including triphenylphosphine, tri-(o-tolyl)phosphine, tri(cyclohexyl)phosphine, BINAP (2,2′-bis-(diphenylphosphino)-1,1′-binaphthyl) or Buchwald's phosphines. The ligand may be present in the palladium compound or added seperately. Examples of suitable palladium compounds include tris(dibenzylideneacetone)dipalladium(0), palladium(II)-bis(o-tolyl)phosphine chloride and palladium(II) acetate.

Usually, the reaction is performed in an inert organic solvent. Suitable organic solvents are aromatic hydrocarbons like benzene or toluene, halogenated aromatic hydrocarbons like chlorobenzene, halogenated hydrocarbons like dichloromethane, trichloromethane, dichloroethane, ethers such as tetrahydrofuran, dioxane, ethyleneglycoldimethylether, methyl tert-butylether, or amides like dimethylformamide or N-methylpyrrolidone, and the mixtures thereof.

The Buchwald-Hartwig reaction is typically run in a temperature range from −20° C. to 150° C., preferably from 20 to 80° C.

The reaction can be conducted under normal conditions or under micro-wave conditions, see e.g. K. Loones, B. Maes, G. Rombouts, S. Hostyn, G. Diels Tetrahedron 2005, 61, 10338-10348.

The amine compounds II are known from the literature, see e.g. U.S. Pat. No. 6,127,386, U.S. Pat. No. 6,437,138, WO 94 08992, WO 96/40682, WO 97/46554, WO 98/25920, WO 99/32480), or they can be prepared according to the methods described therein. Compounds of the formula II can also be prepared as outlined in scheme 4.

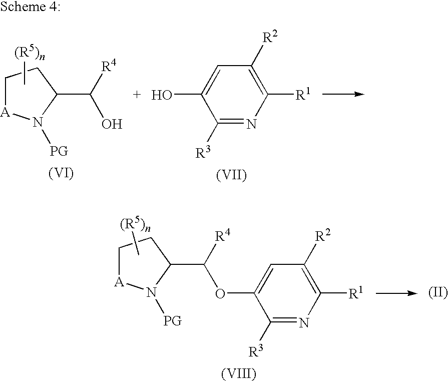

In scheme 4, n, R¹, R², R³, R⁴ and R⁵ are as defined above. PG is conventional protecting group, e.g. an urethane protecting group such as BOC or CBZ. According to the method of scheme 4, a hydroxymethyl compound of the general formula VI is reacted with 3-hydroxypyridines of the formula VII under Mitsunobu conditions, e.g. in the presence of a triaryl phosphine compound such as triphenyl phosphine in the presence of a azodicarboxylate ester such as diethyl azodicarboxylate (DEAD). Thereby compounds of the formula VIII are obtained, which can be deprotected to obtain compounds of the formula II. The reaction can be performed by analogy to conventional Mitsunobu reaction as described in Org. React. 1992, 42, pp. 335-656, Synthesis 1981, pp. 1-28, J. Med. Chem. 1996, 39, pp. 817-825.

Pyridine N-oxides of II can be obtained by oxidizing compounds of the general formula VIII by suitable oxidizing agents such as organic peracids (for details see Katritzky et al. “Chemistry of Heterocyclic N-oxides pp. 21-72, 539-542, Academic Press, New York 1971 and the literature cited therein) and thereafter removing the protective group R.

The compounds VI can be synthesized according to know procedures, for details see Tetrahedron Lett. 42 2001 8247, Tetrahedron lett 30 11 1331,Tetrahedron Lett. 30 11 1327 1989, Tetrahedron, Asym., 9, 1998, 1935 and Houben Weyl (Thieme 1990) Vol. E16a, p 84 ff and p 304 ff.

The hydroxypyridines VII are commercially available or can be synthesized according to the literature cited for the preparation of compounds II.

Optionally, compounds I or compounds II, respectively, wherein R¹ and R² are halogen can be converted into further derivatives I or II, respectively by substitution of R¹ and/or R² with nucleophiles like amines (see e.g., Chem. Ber. 1969, 102, p. 1161) thiols (see e.g., Tetrahedron 1985, 41, p. 1373, Tetrahedron 1983, p. 4153), alkoxides (see e.g., Tetrahedron 1992, 48, p. 3633), a boronic acid under Suzuki conditions (see e.g., J. Org. Chem. 67, 2002, p. 5588, U.S. Pat. No. 6,127,386,U.S. Pat. No. 6,437,138), a tinalkyl or tinalkenyl under Stille conditions (see e.g., Monatshefte Chem. 1995, 126, p. 805) an acetylene under Sonogashira conditions (see e.g., Tetrahedron Asym. 12, 2001 p. 1121, J. Org. Chem. 68, 2003, p. 1571, J. Med. Chem. 2005, 48, p. 1721, U.S. Pat. No. 6,127,386) or a cyanide source (see e.g., J. Chem. Res., Synop. 2003, 12, p. 814, Eur J. Chem. 2003, 9 (8), pp. 1828.

Compounds I or II, respectively, wherein R² is a heterocyclic ring can be obtained according to methods described in the art, for example in Joule, Mills, “Heterocyclic Chemistry”, Chapman+Hill 2000; Katritzky, Rees“Comprehensive Heterocyclic Chemistry”, Vol. 1-8, Pergamon Press 1984;“The Chemistry of Heterocyclic Compounds”. Wiley, Vol. 14 (1974), Vol. 29 (1974), 34 (1979), 37 (1981), 41 (1982), 49 (1991) or Houben Weyl, “The Science of Synthesis”, Vol 9-22, Thieme 2005; or in references cited therein.

Compounds of the formulae I or II, respectively, with y being one can be obtained by oxidizing compounds of the general formulae I or II, respectively, with y being zero with suitable oxidizing agents such as organic peracids (for details see Katritzky et al. “Chemistry of Heterocyclic N-oxides pp. 21-72, 539-542, Academic Press, New York 1971 and the literatur cited therein, Recl. Tray. Pays-Bas, 1957, 76, p. 58, Synth. Comm. 2000, 30(8), p. 1529, J. Het. Chem. 1996, 33 (4), p. 1051).

After completion of the reaction, the compound of formula I can be isolated by employing conventional methods such as adding the reaction mixture to water, extracting with an organic solvent, concentrating the extract and the like. The isolated compound I can be purified by a technique such as chromatography, recrystallization and the like, if necessary.

If individual compounds I are not obtainable by the routes described above, they can be prepared by derivatization of other compounds I or by customary modifications of the synthesis routes described.

The preparation of the compounds of formula I may lead to them being obtained as isomer mixtures (stereoisomers, enantiomers). If desired, these can be resolved by the methods customary for this purpose, such as crystallization or chromatography, also on optically active adsorbate, to give the pure isomers. In some cases the individual isomers will be interconverted during work-up for use or during application (for example under the action of light, acids or bases). Such conversions may also take place after use.

A skilled person will readily understand that the pure isomers can be obtained when using optical active compounds as starting materials.

Agronomically acceptable salts of the compounds I can be formed in a customary manner, e.g. by reaction with an acid of the anion in question.

The compounds of the formula I are especially suitable for efficiently combating pest, in particular arthropod pests and nemathode pest. The compounds of the formula I according to the present invention are particularly suitable for controlling/combating insect pests. The compounds of the formula I according to the present invention are particularly suitable for controlling/combating arachnid pests.

The compounds are particularly suitable for controlling pests of the following orders:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis,

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga sp., Simulium vittatum, Stomoxys calcitrans, Tabanus bovinus, Tabanus atratus, Tabanus lineola, and Tabanus similis, Tipula oleracea, and Tipula paludosa

thrips (Thysanoptera), e.g. Dichromothrips corbetti, Dichromothrips ssp, Frankliniella fusca, Frankliniella occidentalis, Frankliniella tritici, Scirtothrips citri, Thrips oryzae, Thrips palmi and Thrips tabaci,

termites (Isoptera), e.g. Calotermes flavicollis, Leucotermes flavipes, Heterotermes aureus, Reticulitermes flavipes, Reticulitermes virginicus, Reticulitermes lucifugus, Termes natalensis, and Coptotermes formosanus,

cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla pin, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus.

ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,

crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina,

arachnids (Acarina or Acari), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa,

fleas (Siphonaptera), e.g. Ctenocephalides felis, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Scutigera coleoptrata,

millipedes (Diplopoda), e.g. Narceus spp.,

earwigs (Dermaptera), e.g. forficula auricularia,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus,

Plant parasitic nematodes such as root-knot nematodes, Meloidogyne arenaria, Meloidogyne chitwoodi, Meloidogyne exigua, Meloidogyne hapla, Meloidogyne incognita, Meloidogyne javanica and other Meloidogyne species; cyst nematodes, Globodera rostochiensis, Globodera pallida, Globodera tabacum and other Globodera species, Heterodera avenae, Heterodera glycines, Heterodera schachtii, Heterodera trifolii, and other Heterodera species; seed gall nematodes, Anguina funesta, Anguina tritici and other Anguina species; stem and foliar nematodes, Aphelenchoides besseyi, Aphelenchoides fragariae, Aphelenchoides ritzemabosi and other Aphelenchoides species; sting nematodes, Belonolaimus longicaudatus and other Belonolaimus species; pine nematodes, Bursaphelenchus xylophilus and other Bursaphelenchus species; ring nematodes, Criconema species, Criconemella species, Criconemoides species, and Mesocriconema species; stem and bulb nematodes, Ditylenchus destructor, Ditylenchus dipsaci, Ditylenchus myceliophagus and other Ditylenchus species; awl nematodes, Dolichodorus species; spiral nematodes, Helicotylenchus dihystera, Helicotylenchus multicinctus and other Helicotylenchus species, Rotylenchus robustus and other Rotylenchus species; sheath nematodes, Hemicycliophora species and Hemicriconemoides species; Hirshmanniella species; lance nematodes, Hoplolaimus columbus, Hoplolaimus galeatus and other Hoplolaimus species; false root-knot nematodes, Nacobbus aberrans and other Nacobbus species; needle nematodes, Longidorus elongates and other Longidorus species; pin nematodes, Paratylenchus species; lesion nematodes, Pratylenchus brachyurus, Pratylenchus coffeae, Pratylenchus curvitatus, Pratylenchus goodeyi, Pratylencus neglectus, Pratylenchus penetrans, Pratylenchus scribneri, Pratylenchus vulnus, Pratylenchus zeae and other Pratylenchus species; Radinaphelenchus cocophilus and other Radinaphelenchus species; burrowing nematodes, Radopholus similis and other Radopholus species; reniform nematodes, Rotylenchulus reniformis and other Rotylenchulus species; Scutellonema species; stubby root nematodes, Trichodorus primitivus and other Trichodorus species; Paratrichodorus minor and other Paratrichodorus species; stunt nematodes, Tylenchorhynchus claytoni, Tylenchorhynchus dubius and other Tylenchorhynchus species and Merlinius species; citrus nematodes, Tylenchulus semipenetrans and other Tylenchulus species; dagger nematodes, Xiphinema americanum, Xiphinema index, Xiphinema diversicaudatum and other Xiphinema species; and other plant parasitic nematode species.

The formulations are prepared in a known manner (see e.g. for review U.S. Pat. No. 3,060,084, EP-A 707 445 (for liquid concentrates), Browning,” Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7515-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and binders.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.

Examples of suitable carriers are ground natural minerals (for example kaolins, clays, talc, chalk) and ground synthetic minerals (for example highly disperse silica, silicates).

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

The compounds of formula I can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound(s) may also be used successfully in the ultra-low-volume process (ULV), it being possible to apply formulations comprising over 95% by weight of active compound, or even to apply the active compound without additives.

The following are examples of formulations:

1. Products for dilution with water for foliar applications.

For seed treatment purposes, such products may be applied to the seed diluted or undiluted.

### A) Water-Soluble Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formulation with 10% (w/w) of active compound(s) is obtained.

### B) Dispersible Concentrates (DC)

20 parts by weight of the active compound(s) are dissolved in 75 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

### C) Emulsifiable Concentrates (EC)

15 parts by weight of the active compound(s) are dissolved in 75 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

### D) Emulsions (EW, EO, ES)

40 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

### E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

### F) Water-Dispersible Granules and Water-Soluble Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

### G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

2. Products to be applied undiluted for foliar applications. For seed treatment purposes, such products may be applied to the seed diluted or undiluted.

### H) Dustable Powders (DP, DS)

5 parts by weight of the active compound(s) are ground finely and mixed intimately with 95 parts by weight of finely divided kaolin. This gives a dustable product having 5% (w/w) of active compound(s)

### I) Granules (GR, FG, GG, MG)

0.5 part by weight of the active compound(s) is ground finely and associated with 95.5 parts by weight of carriers, whereby a formulation with 0.5% (w/w) of active compound(s) is obtained. Current methods are extrusion, spray-drying or the fluidized bed. This gives granules to be applied undiluted for foliar use.

### J) ULV Solutions (UL, LS)

10 parts by weight of the active compound(s) are dissolved in 90 parts by weight of an organic solvent, for example xylene. This gives a product having 10% (w/w) of active compound(s), which is applied undiluted for foliar use.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds of formula I are effective through both contact and ingestion. The compounds of formula I are also suitable for the protection of the seed, plant propagules and the seedlings' roots and shoots, preferably the seeds, against soil pests and also for the treatment plant seeds which tolerate the action of herbicides or fungicides or insecticides owing to breeding, including genetic engineering methods.

Conventional seed treatment formulations include for example flowable concentrates FS, solutions LS, powders for dry treatment DS, water dispersible powders WS or granules for slurry treatment, water soluble powders SS and emulsion ES. Application to the seeds is carried out before sowing, either directly on the seeds.

The seed treatment application of the compounds of formula I or formulations containing them is carried out by spraying or dusting the seeds before sowing of the plants and before emergence of the plants.

The invention also relates to the propagation product of plants, and especially the treated seed comprising, that is, coated with and/or containing, a compound of formula I or a composition comprising it. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

The seed comprises the inventive compounds or compositions comprising them in an amount of from 0.1 g to 10 kg per 100 kg of seed.

Compositions of this invention may also contain other active ingredients, for example other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

The following list of pesticides together with which the compounds according to the invention can be used, is intended to illustrate the possible combinations, but not to impose any limitation:

A.1. Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;

A.2. Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;

A.3. Pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;

A.4. Growth regulators: a) chitin synthesis inhibitors: benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentezine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;

A.5. Nicotinic receptor agonists/antagonists compounds: clothianidin, dinotefuran, imidacloprid, thiamethoxam, nitenpyram, acetamiprid, thiacloprid;

the thiazol compound of formula (Γ¹)

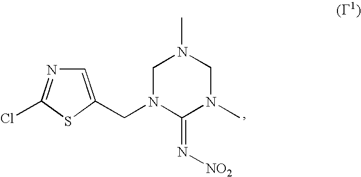

A.6. GABA antagonist compounds: acetoprole, endosulfan, ethiprole, fipronil, vaniliprole, pyrafluprole, pyriprole, the phenylpyrazole compound of formula Γ²

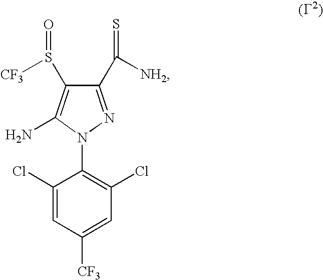

A.7. Macrocyclic lactone insecticides: abamectin, emamectin, milbemectin, lepimectin, spinosad;

A.8. METI I compounds: fenazaquin, pyridaben, tebufenpyrad, tolfenpyrad, flufenerim;

A.9. METI II and III compounds: acequinocyl, fluacyprim, hydramethylnon;

A.10. Uncoupler compounds: chlorfenapyr;

A.11. Oxidative phosphorylation inhibitor compounds: cyhexatin, diafenthiuron, fenbutatin oxide, propargite;

A.12. Moulting disruptor compounds: cyromazine;

A.13. Mixed Function Oxidase inhibitor compounds: piperonyl butoxide;

A.14. Sodium channel blocker compounds: indoxacarb, metaflumizone,

A.15. Various: benclothiaz, bifenazate, cartap, flonicamid, pyridalyl, pymetrozine, sulfur, thiocyclam, flubendiamide, cyenopyrafen, flupyrazofos, cyflumetofen, amidoflumet,

N—R′-2,2-dihalo-1-R″cyclo-propanecarboxamide-2-(2,6-dichloro-alpha,alpha,alpha-tri-fluoro-p-tolyl)hydrazone or N—R′-2,2-di(R′″)propionamide-2-(2,6-dichloro-alpha,alpha,alpha-trifluoro-p-tolyl)-hydrazone, wherein R′ is methyl or ethyl, halo is chloro or bromo, R″ is hydrogen or methyl and R′″ is methyl or ethyl, anthranilamide compounds of formula Γ³

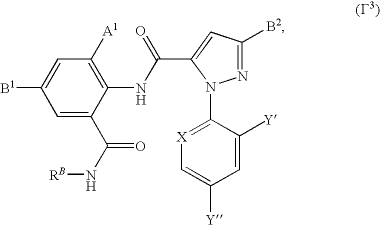

wherein A¹ is CHs, Cl, Br, I, X is C—H, C—Cl, C—F or N, Y′ is F, Cl, or Br, Y″ is F, Cl, CF₃, B¹ is hydrogen, Cl, Br, I, CN, B² is Cl, Br, CF₃, OCH₂CF₃, OCF₂H, and R^{B} is hydrogen, CHs or CH(CH₃)₂, and malononitrile compounds as described in JP 2002 284608, WO 02/89579, WO 02/90320, WO 02/90321, WO 04/06677, WO 04/20399, or JP 2004-99597.

The commercially available compounds of the group A may be found in The Pesticide Manual, 13^{th} Edition, British Crop Protection Council (2003) among other publications.

Thiamides of formula Γ² and their preparation have been described in WO 98/28279.

Lepimection is known from Agro Project, PJB Publications Ltd, November 2004. Benclothiaz and its preparation have been described in EP-A1 454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1 462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. Cyflumetofen and its preparation have been described in WO 04/080180.

The pest may be controlled by contacting the target parasite/pest, its food supply, habitat, breeding ground or its locus with a pesticidally effective amount of compounds of or compositions of formula I.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds or compositions of the invention can also be applied preventively to locuses at which occurrence of the pests is expected.

The compounds of formula I may also be used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

For use in treating crop plants, the rate of application of the active ingredients of this invention may be in the range of 0.1 g to 4000 g per hectare, desirably from 25 g to 600 g per hectare, more desirably from 50 g to 500 g per hectare.

The compounds of the present invention may also be applied to seeds.

### SYNTHESIS EXAMPLES

With due modification of the starting compounds, the protocols shown in the synthesis example below were used for obtaining further compounds I. The resulting compounds, together with physical data, are listed in table 1 below.

The products were characterized by coupled High Performance Liquid Chromatography/mass spectrometry (HPLC/MS), by ¹H-NMR (400 MHz) in CDCl₃ or by their melting points. HPLC column: RP-18 column (Chromolith Speed ROD from Merck KgaA, Germany). Elution: acetonitrile+0.1% trifluoroacetic acid (TFA)/water in a ratio of from 5:95 to 95:5 in 5 minutes at 40° C. MS: Quadrupol electrospray ionisation, 80 V (positiv modus). The m/z value given correlates to the molecule peak containing the lightest isotops of all atoms.

### Example Ia-155

### 3-Chloro-5-((S)-1-isopropyl-azetidin-2-ylmethoxy)-pyridine

Route a:

A solution of 3-((S)-1-azetidin-2-ylmethoxy)-5-chloro-pyridine (1 eq), 2-bromopropane (1.05 eq) and diisopropylamine (1.10 eq) in acetonitrile (concentration of amine 45 mmol/L) was heated to reflux over 24 hours. After cooling, all volatile components were removed under reduced pressure and the residual mixture was directly purified on silicagel. 3-Chloro-5-((S)-1-isopropyl-azetidin-2-ylmethoxy)-pyridine was obtained with a high purity (>95%).

Route b:

To a solution of 3-((S)-1-azetidin-2-ylmethoxy)-5-chloro-pyridine (1 eq), acetone (1.00 eq) in tetrahydrofuran (concentration of amine 80 mmol/L) was added supported triacetoxyborohydride (3 eq). The mixture was shaken 3 days, filtrated and the solid phase washed with tetrahydrofuran. After removal of solvents and purification by chromatography, 3-chloro-5-((S)-1-isopropyl-azetidin-2-ylmethoxy)-pyridine was obtained with a high purity (>95%).

¹H-NMR (CDCl₃, 500 MHz): 8.2 (s, 2H), 7.2 (s, 1H), 4.2 (m, 1H), 4.0 (m, 1H), 3.65 (m, 1H), 3.55 (m, 1H), 3.0 (m, 1H), 2.6 (m, 1H), 2.1 (m, 2H), 1.08 (d, 3H), 1.0 (d, 3H).

### Example Ia-163

### 3-Chloro-5-((S)-1-phenyl-azetidin-2-ylmethoxy)-pyridine

A solution of 3-((S)-1-azetidin-2-ylmethoxy)-5-chloro-pyridine (1 eq), bromobenzene (1.00 eq), potassium tert-butanol (2.10 eq), Pd(OAc)₂ and (+/−)Binap in toluene (concentration of amine 45 mmol/L) was heated at 80° C. over 1 hour. After evaporation of the solvent, the residue was purified by chromatography to give 3-chloro-5-((S)-1-phenyl-azetidin-2-ylmethoxy)-pyridine in a high purity (>95%).

¹H-NMR (CDCl₃, 500 MHz): 8.3 (s, 1H), 8.2 (s, 1H), 7.25 (m, 3H), 6.8 (m, 1H), 6.6 (m, 2H), 4.4 (m, 1H), 4.25 (m, 2H), 4.0 (m, 1H), 3.7 (m, 1H), 2.4 (m, 2H).

### Example Ib-3

### 3-Chloro-5-((S)-1-isopropyl-pyrrolidin-2-ylmethoxy)-pyridine

A solution of 3-((S)-1-pyrrolidin-2-ylmethoxy)-5-chloro-pyridine (1 eq), 2-bromopropane (1.10 eq) and diisopropylamine (1.10 eq) in acetonitrile (concentration of amine 140 mmol/L) was heated to reflux over 24 hours. After cooling, all volatile components were removed under reduced pressure and the residual mixture was directly purified on silicagel. 3-Chloro-5-((S)-1-isopropyl-azetidin-2-ylmethoxy)-pyridine was obtained with a high purity (>95%).

¹H-NMR (CDCl₃, 500 MHz): 8.18 (s, 1H), 8.15 (s, 1H), 7.2 (s, 1H), 3.9 (m, 1H), 3.7 (m, 1H), 3.2 (m, 1H), 3.0 (m, 2H), 2.6 (m, 1H), 1.9 (m, 1H), 1.8 (m, 2+1H), 1.17 (d, 3H), 1.08 (d, 3H).

Using essentially the same procedure as described in examples Ia-155, Ib-163 or Ib-3, the compounds shown in table I or table II, respectively, were prepared and identified by ¹H-NMR or MS spectral analysis or melting point.

In tables I and II, the letter given in column C* gives the configuration at the indicated carbon atom.

TABLE I
Compounds of the formula Ia (= compound of formula I with A
being a chemical bond, n = 0, y = 0, and R³ is hydrogen)

| | |
|---|---|
| | (Ia-S) |
| 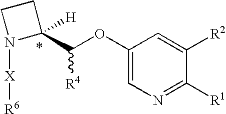 | |
| | (Ia-R) |
| 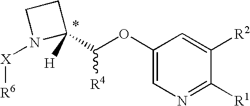 | |

| Ex. | C* | R¹ | R² | R⁴ | X—R⁶ | Physical Data |
|---|---|---|---|---|---|---|
| Ia-1 | S | H | Cl | H | 2-methyl-propyl | RT = 1.76 min, m/z = 255 |
| | | | | | | [M + H]⁺ |
| Ia-2 | S | H | Cl | H | cyclopropyl-methyl | RT = 1.63 min, m/z = 253 |
| | | | | | | [M + H]⁺ |
| Ia-3 | S | H | Cl | H | benzyl | RT = 1.93 min, m/z = 289 |
| | | | | | | [M + H]⁺ |
| Ia-4 | S | Cl | H | H | 2-hydroxy-butyl | RT = 1.55 min, m/z = 271 |
| | | | | | | [M + H]⁺ |
| Ia-5 | S | Cl | H | H | 2-acetoxy-butyl | RT = 1.99 min, m/z = 313 |
| | | | | | | [M + H]⁺ |
| Ia-6 | S | Cl | H | H | 4-fluoro-benzyl | RT = 2.03 min, m/z = 307 |
| | | | | | | [M + H]⁺ |
| Ia-7 | S | Cl | H | H | 3-benzyloxy-benzyl | RT = 2.73 min, m/z = 395 |
| | | | | | | [M + H]⁺ |
| Ia-8 | S | Cl | H | H | 3-nitro-benzyl | RT = 2.03 min, m/z = 334 |
| | | | | | | [M + H]⁺ |
| Ia-9 | S | Cl | H | H | 2-benzyloxy-benzyl | RT = 2.72 min, m/z = 395 |
| | | | | | | [M + H]⁺ |
| Ia-10 | S | H | Cl | H | 4-fluoro-benzyl | RT = 2.03 min, m/z = 307 |
| | | | | | | [M + H]⁺ |
| Ia-11 | S | H | Cl | H | 3-benzyloxy-benzyl | RT = 2.72 min, m/z = 395 |
| | | | | | | [M + H]⁺ |
| Ia-12 | S | H | Cl | H | 3-nitro-benzyl | RT = 1.97 min, m/z = 334 |
| | | | | | | [M + H]⁺ |
| Ia-13 | S | H | Cl | H | 2-benzyloxy-benzyl | RT = 2.66 min, m/z = 395 |
| | | | | | | [M + H]⁺ |
| Ia-14 | S | Cl | Cl | H | 4-fluoro-benzyl | RT = 2.44 min, m/z = 341 |
| | | | | | | [M + H]⁺ |
| Ia-15 | S | Cl | Cl | H | 3-nitro-benzyl | RT = 2.38 min, m/z = 368 |
| | | | | | | [M + H]⁺ |
| Ia-16 | S | Cl | H | H | 3-hydroxy-benzyl | RT = 1.75 min, m/z = 305 |
| | | | | | | [M + H]⁺ |
| Ia-17 | S | Cl | H | H | 2-benzyloxy-benzyl | RT = 2.77 min, m/z = 395 |
| | | | | | | [M + H]⁺ |
| Ia-18 | S | Cl | H | H | 4-amino-benzyl | RT = 1.36 min, m/z = 304 |
| | | | | | | [M + H]⁺ |
| Ia-19 | S | Cl | H | H | 2,5-dichloro-benzyl | RT = 2.31 min, m/z = 357 |
| | | | | | | [M + H]⁺ |
| Ia-20 | S | Cl | H | H | 2-hydroxy-benzyl | RT = 1.89 min, m/z = 305 |
| | | | | | | [M + H]⁺ |
| Ia-21 | S | Cl | H | H | 4-acetylamino-benzyl | RT = 1.66 min, m/z = 346 |
| | | | | | | [M + H]⁺ |
| Ia-22 | S | Cl | H | H | 4-N,N-dimethylamino- | RT = 1.62 min, m/z = 332 |
| | | | | | benzyl | [M + H]⁺ |
| Ia-23 | S | Cl | H | H | 2-methyl-benzyl | RT = 2.16 min, m/z = 303 |
| | | | | | | [M + H]⁺ |
| Ia-24 | S | H | Cl | H | 3-hydroxy-benzyl | RT = 1.69 min, m/z = 305 |
| | | | | | | [M + H]⁺ |
| Ia-25 | S | H | Cl | H | 2-benzyloxy-benzyl | RT = 2.72 min, m/z = 395 |
| | | | | | | [M + H]⁺ |
| Ia-26 | S | H | Cl | H | 4-methylsulfanyl-benzyl | RT = 2.23 min, m/z = 335 |
| | | | | | | [M + H]⁺ |
| Ia-27 | S | H | Cl | H | 4-amino-benzyl | RT = 1.33 min, m/z = 304 |
| | | | | | | [M + H]⁺ |
| Ia-28 | S | H | Cl | H | 2,5-dichloro-benzyl | RT = 2.26 min, m/z = 357 |
| | | | | | | [M + H]⁺ |
| Ia-29 | S | H | Cl | H | 2-hydroxy-benzyl | RT = 1.84 min, m/z = 305 |
| | | | | | | [M + H]⁺ |
| Ia-30 | S | H | Cl | H | 4-acetylamino-benzyl | RT = 1.61 min, m/z = 346 |
| | | | | | | [M + H]⁺ |
| Ia-31 | S | H | Cl | H | 4-N,N-dimethylamino- | RT = 1.62 min, m/z = 332 |
| | | | | | benzyl | [M + H]⁺ |
| Ia-32 | S | H | Cl | H | 2-methyl-benzyl | RT = 2.11 min, m/z = 303 |
| | | | | | | [M + H]⁺ |
| Ia-33 | S | Cl | Cl | H | 3-hydroxy-benzyl | RT = 2.10 min, m/z = 339 |
| | | | | | | [M + H]⁺ |
| Ia-34 | S | Cl | Cl | H | 4-benzyloxy-benzyl | RT = 3.00 min, m/z = 429 |
| | | | | | | [M + H]⁺ |
| Ia-35 | S | Cl | Cl | H | 4-methylsulfanyl-benzyl | RT = 2.58 min, m/z = 369 |
| | | | | | | [M + H]⁺ |
| Ia-36 | S | Cl | Cl | H | 4-amino-benzyl | RT = 1.91 min, m/z = 338 |
| | | | | | | [M + H]⁺ |
| Ia-37 | S | Cl | Cl | H | 2-methyl, 4-tert-butyl- | RT = 3.13 min, m/z = 393 |
| | | | | | benzyl | [M + H]⁺ |
| Ia-38 | S | Cl | Cl | H | 2-hydroxy-benzyl | RT = 2.24 min, m/z = 339 |
| | | | | | | [M + H]⁺ |
| Ia-39 | S | Cl | Cl | H | 4-acetylamino-benzyl | RT = 1.97 min, m/z = 380 |
| | | | | | | [M + H]⁺ |
| Ia-40 | S | Cl | Cl | H | 4-N,N-dimethylamino- | RT = 2.04 min, m/z = 366 |
| | | | | | benzyl | [M + H]⁺ |
| Ia-41 | S | Cl | Cl | H | 2-methyl-benzyl | RT = 2.49 min, m/z = 337 |
| | | | | | | [M + H]⁺ |
| Ia-42 | S | Cl | H | H | 4-methylsulfanyl-benzyl | RT = 2.27 min, m/z = 335 |
| | | | | | | [M + H]⁺ |
| Ia-43 | S | Cl | H | H | 2-benzyloxy-butyl | RT = 2.72 min, m/z = 361 |
| | | | | | | [M + H]⁺ |
| Ia-44 | S | H | Cl | H | 2-hydroxy-butyl | RT = 1.47 min, m/z = 271 |
| | | | | | | [M + H]⁺ |
| Ia-45 | S | Cl | H | H | 3-chloro-benzyl | RT = 2.25 min, m/z = 323 |
| | | | | | | [M + H]⁺ |
| Ia-46 | S | Cl | H | H | 2-trifluoromethyl- | RT = 2.34 min, m/z = 357 |
| | | | | | benzyl | [M + H]⁺ |
| Ia-47 | S | Cl | H | H | 4-tert-butyl-benzyl | RT = 2.78 min, m/z = 345 |
| | | | | | | [M + H]⁺ |
| Ia-48 | S | Cl | H | H | 2-fluoro-benzyl | RT = 2.01 min, m/z = 307 |
| | | | | | | [M + H]⁺ |
| Ia-49 | S | Cl | H | H | 2,4,6-trimethoxy-benzyl | RT = 2.37 min, m/z = 379 |
| | | | | | | [M + H]⁺ |
| Ia-50 | S | Cl | H | H | 2-naphtalene-methyl | RT = 2.43 min, m/z = 339 |
| | | | | | | [M + H]⁺ |
| Ia-51 | S | Cl | H | H | 4-methanesulfinyl- | RT = 1.57 min, m/z = 351 |
| | | | | | benzyl | [M + H]⁺ |
| Ia-52 | S | Cl | H | H | 4-methoxy-benzyl | RT = 2.08 min, m/z = 319 |
| | | | | | | [M + H]⁺ |
| Ia-53 | S | H | Cl | H | 4-tert-butyl-benzyl | RT = 2.73 min, m/z = 345 |
| | | | | | | [M + H]⁺ |
| Ia-54 | S | H | Cl | H | 2,4,6-trimethoxy-benzyl | RT = 2.30 min, m/z = 379 |
| | | | | | | [M + H]⁺ |
| Ia-55 | S | H | Cl | H | 4-methanesulfinyl- | RT = 1.52 min, m/z = 351 |
| | | | | | benzyl | [M + H]⁺ |
| Ia-56 | S | H | Cl | H | 4-methoxy-benzyl | RT = 2.02 min, m/z = 319 |
| | | | | | | [M + H]⁺ |
| Ia-57 | S | Cl | Cl | H | 2-trifluoromethyl- | RT = 2.66 min, m/z = 391 |
| | | | | | benzyl | [M + H]⁺ |
| Ia-58 | S | Cl | Cl | H | 4-tert-butyl-benzyl | RT = 3.06 min, m/z = 379 |
| | | | | | | [M + H]⁺ |
| Ia-59 | S | Cl | Cl | H | 2,4,6-trimethoxy-benzyl | RT = 2.66 min, m/z = 413 |
| | | | | | | [M + H]⁺ |
| Ia-60 | S | Cl | Cl | H | 2-naphtalene-methyl | RT = 2.71 min, m/z = 373 |
| | | | | | | [M + H]⁺ |
| Ia-61 | S | Cl | Cl | H | 4-hydroxy-benzyl | RT = 2.05 min, m/z = 339 |
| | | | | | | [M + H]+ |
| Ia-62 | S | Cl | Cl | H | 4-methanesulfinyl- | RT = 1.91 min, m/z = 385 |
| | | | | | benzyl | [M + H]⁺ |
| Ia-63 | S | Cl | Cl | H | 4-methoxy-benzyl | RT = 2.41 min, m/z = 353 |
| | | | | | | [M + H]⁺ |
| Ia-64 | S | Cl | H | H | 2,4-dichloro-benzyl | RT = 2.39 min, m/z = 357 |
| | | | | | | [M + H]⁺ |
| Ia-65 | S | Cl | H | H | 4-chloro-benzyl | RT = 2.25 min, m/z = 323 |
| | | | | | | [M + H]⁺ |
| Ia-66 | S | Cl | H | H | 2-methoxy-benzyl | RT = 2.13 min, m/z = 319 |
| | | | | | | [M + H]⁺ |
| Ia-67 | S | Cl | H | H | 3-methyl-benzyl | RT = 2.19 min, m/z = 303 |
| | | | | | | [M + H]⁺ |
| Ia-68 | S | Cl | H | H | 3-trifluoromethyl- | RT = 2.40 min, m/z = 357 |
| | | | | | benzyl | [M + H]⁺ |
| Ia-69 | S | H | Cl | H | 2,4-dichloro-benzyl | RT = 2.37 min, m/z = 357 |
| | | | | | | [M + H]⁺ |
| Ia-70 | S | H | Cl | H | 4-chloro-benzyl | RT = 2.23 min, m/z = 323 |
| | | | | | | [M + H]⁺ |
| Ia-71 | S | H | Cl | H | 2-methoxy-benzyl | RT = 2.11 min, m/z = 319 |
| | | | | | | [M + H]⁺ |
| Ia-72 | S | H | Cl | H | 3-methyl-benzyl | RT = 2.18 min, m/z = 303 |
| | | | | | | [M + H]⁺ |
| Ia-73 | S | H | Cl | H | 3-trifluoromethyl- | RT = 2.38 min, m/z = 357 |
| | | | | | benzyl | [M + H]⁺ |
| Ia-74 | S | H | Cl | H | 2,6-dichloro-benzyl | RT = 2.15 min, m/z = 357 |
| | | | | | | [M + H]⁺ |
| Ia-75 | S | H | Cl | H | 2-chloro, 6-methane- | RT = 2.29 min, m/z = 369 |
| | | | | | sulfinyl-benzyl | [M + H]⁺ |
| Ia-76 | S | H | Cl | H | 3-methoxy-benzyl | RT = 2.05 min, m/z = 319 |
| | | | | | | [M + H]⁺ |
| Ia-77 | S | H | Cl | H | 4-nitro-benzyl | RT = 2.01 min, m/z = 334 |
| | | | | | | [M + H]⁺ |
| Ia-78 | S | H | Cl | H | 3-fluoro-benzyl | RT = 2.03 min, m/z = 307 |
| | | | | | | [M + H]⁺ |
| Ia-79 | S | Cl | Cl | H | 2,4-dichloro-benzyl | RT = 2.73 min, m/z = 392 |
| | | | | | | [M + H]⁺ |
| Ia-80 | S | Cl | Cl | H | 4-chloro-benzyl | RT = 2.60 min, m/z = 357 |
| | | | | | | [M + H]⁺ |
| Ia-81 | S | Cl | Cl | H | 2-methoxy-benzyl | RT = 2.50 min, m/z = 353 |
| | | | | | | [M + H]⁺ |
| Ia-82 | S | Cl | Cl | H | 3-methyl-benzyl | RT = 2.55 min, m/z = 337 |
| | | | | | | [M + H]⁺ |
| Ia-83 | S | Cl | Cl | H | 3-trifluoromethyl- | RT = 2.74 min, m/z = 391 |
| | | | | | benzyl | [M + H]⁺ |
| Ia-84 | S | Cl | Cl | H | 2,6-dichloro-benzyl | RT = 2.54 min, m/z = 392 |
| | | | | | | [M + H]⁺ |
| Ia-85 | S | Cl | Cl | H | 2-chloro, 6-methane- | RT = 2.65 min, m/z = 404 |
| | | | | | sulfinyl-benzyl | [M + H]⁺ |
| Ia-86 | S | Cl | Cl | H | 3-methoxy-benzyl | RT = 2.42 min, m/z = 353 |
| | | | | | | [M + H]⁺ |
| Ia-87 | S | Cl | Cl | H | 4-nitro-benzyl | RT = 2.40 min, m/z = 368 |
| | | | | | | [M + H]⁺ |
| Ia-88 | S | Cl | Cl | H | 3-fluoro-benzyl | RT = 2.43 min, m/z = 341 |
| | | | | | | [M + H]⁺ |
| Ia-89 | S | Cl | H | H | 2-chloro-benzyl | RT = 2.18 min, m/z = 323 |
| | | | | | | [M + H]⁺ |
| Ia-90 | S | H | Cl | H | 2-chloro-benzyl | RT = 2.10 min, m/z = 323 |
| | | | | | | [M + H]⁺ |
| Ia-91 | S | Cl | Cl | H | 2-chloro-benzyl | RT = 2.51 min, m/z = 357 |
| | | | | | | [M + H]⁺ |
| Ia-92 | S | Cl | H | H | 3-amino-benzyl | RT = 1.43 min, m/z = 304 |
| | | | | | | [M + H]⁺ |
| Ia-93 | S | Cl | H | H | 4-hydroxy-benzyl | RT = 1.70 min, m/z = 305 |
| | | | | | | [M + H]⁺ |
| Ia-94 | S | H | Cl | H | 3,5-di-tert-butyl-4- | RT = 3.03 min, m/z = 417 |
| | | | | | hydroxy-benzyl | [M + H]⁺ |
| Ia-95 | S | H | Cl | H | 4-hydroxy-benzyl | RT = 1.65 min, m/z = 305 |
| | | | | | | [M + H]⁺ |
| Ia-96 | S | Cl | H | H | 4-methyl-benzyl | RT = 2.22 min, m/z = 303 |
| | | | | | | [M + H]⁺ |
| Ia-97 | S | Cl | H | H | 3-trifluoromethoxy- | RT = 2.51 min, m/z = 373 |
| | | | | | benzyl | [M + H]⁺ |
| Ia-98 | S | Cl | H | H | 3-chloro, 6-methane- | RT = 2.81 min, m/z = 415 |
| | | | | | sulfinyl-benzyl | [M + H]⁺ |
| Ia-99 | S | Cl | H | H | 2-fluoro, 4-bromo- | RT = 2.33 min, m/z = 385 |
| | | | | | benzyl | [M + H]⁺ |
| Ia-100 | S | Cl | H | H | 4-amino-benzyl | RT = 1.91 min, m/z = 304 |
| | | | | | | [M + H]⁺ |
| Ia-101 | S | Cl | H | H | 3-bromo, 6-fluoro- | RT = 2.27 min, m/z = 385 |
| | | | | | benzyl | [M + H]⁺ |
| Ia-102 | S | Cl | H | H | 2-diphenylphosphino- | RT = 3.27 min, m/z = 473 |
| | | | | | benzyl | [M + H]⁺ |
| Ia-103 | S | Cl | H | H | 2,3,4,5,6-pentamethyl- | RT = 2.73 min, m/z = 359 |
| | | | | | benzyl | [M + H]⁺ |
| Ia-104 | S | Cl | H | H | 3-bromo, 4-fluoro- | RT = 2.35 min, m/z = 385 |
| | | | | | benzyl | [M + H]⁺ |
| Ia-105 | S | Cl | H | H | 2-methanesulfanyl, | RT = 2.78 min, m/z = 442 |
| | | | | | 5-nitro-benzyl | [M + H]⁺ |
| Ia-106 | S | Cl | H | H | 1-naphtalene-methyl | RT = 2.37 min, m/z = 339 |
| | | | | | | [M + H]⁺ |
| Ia-107 | S | H | Cl | H | 4-methyl-benzyl | RT = 2.17 min, m/z = 303 |
| | | | | | | [M + H]+ |
| Ia-108 | S | H | Cl | H | 3-trifluoromethoxy- | RT = 2.47 min, m/z = 373 |
| | | | | | benzyl | [M + H]⁺ |
| Ia-109 | S | H | Cl | H | 3-fluoro, 6-methane- | RT = 2.77 min, m/z = 415 |
| | | | | | sulfanyl-benzyl | [M + H]⁺ |
| Ia-110 | S | H | Cl | H | 2-fluoro, 4-bromo- | RT = 2.28 min, m/z = 385 |
| | | | | | benzyl | [M + H]⁺ |
| Ia-111 | S | H | Cl | H | 4-amino-benzyl | RT = 1.85 min, m/z = 304 |
| | | | | | | [M + H]⁺ |
| Ia-112 | S | H | Cl | H | 3-bromo, 6-fluoro- | RT = 2.20 min, m/z = 385 |
| | | | | | benzyl | [M + H]⁺ |
| Ia-113 | S | H | Cl | H | 2,3,4,5,6-penta-methyl- | RT = 2.68 min, m/z = 359 |
| | | | | | benzyl | [M + H]⁺ |
| Ia-114 | S | H | Cl | H | 3-bromo, 4-fluoro- | RT = 2.29 min, m/z = 385 |
| | | | | | benzyl | [M + H]⁺ |
| Ia-115 | S | H | Cl | H | 2-methanesulfanyl, | RT = 2.72 min, m/z = 442 |
| | | | | | 5-nitro-benzyl | [M + H]⁺ |
| Ia-116 | S | H | Cl | H | 1-naphtalene-methyl | RT = 2.30 min, m/z = 339 |
| | | | | | | [M + H]⁺ |
| Ia-117 | S | Cl | H | H | 2,3-dihydroxy-propyl | RT = 1.31 min, m/z = 273 |
| | | | | | | [M + H]⁺ |
| Ia-118 | S | H | Cl | H | 2-benzyloxy-butyl | RT = 2.62 min, m/z = 361 |
| | | | | | | [M + H]⁺ |
| Ia-119 | S | Cl | H | H | 2,6-dichloro-benzyl | RT = 2.18 min, m/z = 357 |
| | | | | | | [M + H]⁺ |
| Ia-120 | S | Cl | H | H | 2-chloro, 6-methane- | RT = 2.31 min, m/z = 369 |
| | | | | | sulfinyl-benzyl | [M + H]⁺ |
| Ia-121 | S | Cl | H | H | 3-methoxy-benzyl | RT = 2.07 min, m/z = 319 |
| | | | | | | [M + H]⁺ |
| Ia-122 | S | Cl | H | H | 4-nitro-benzyl | RT = 2.03 min, m/z = 334 |
| | | | | | | [M + H]⁺ |
| Ia-123 | S | Cl | H | H | 3-fluoro-benzyl | RT = 2.06 min, m/z = 307 |
| | | | | | | [M + H]⁺ |
| Ia-124 | S | Cl | H | H | 2-[1-phenyl-meth-(E)- | RT = 3.17 min, m/z = 385 |
| | | | | | ylidene]-heptyl | [M + H]⁺ |
| Ia-125 | S | Cl | H | H | 3,5,5-trimethyl-hexyl | RT = 2.88 min, m/z = 325 |
| | | | | | | [M + H]⁺ |
| Ia-126 | S | Cl | H | H | 3,3-dimethyl-butyl | RT = 2.27 min, m/z = 283 |
| | | | | | | [M + H]⁺ |
| Ia-127 | S | Cl | H | H | undecyl | RT = 3.47 min, m/z = 353 |
| | | | | | | [M + H]⁺ |
| Ia-128 | S | Cl | H | H | 3-methyl-butyl | RT = 2.09 min, m/z = 269 |
| | | | | | | [M + H]⁺ |
| Ia-129 | S | Cl | H | H | 2-(2,6,6-trimethyl- | RT = 2.98 min, m/z = 349 |
| | | | | | cyclohex-1-enyl)-ethyl | [M + H]⁺ |
| Ia-130 | S | Cl | H | H | (Z)-dec-4-enyl | RT = 3.09 min, m/z = 337 |
| | | | | | | [M + H]⁺ |
| Ia-131 | S | Cl | H | H | 3-methanesulfanyl- | RT = 1.86 min, m/z = 287 |
| | | | | | propyl | [M + H]⁺ |
| Ia-132 | S | Cl | H | H | 2-ethyl-hexyl | RT = 2.70 min, m/z = 311 |
| | | | | | | [M + H]⁺ |
| Ia-133 | S | H | Cl | H | 2-[1-phenyl-meth-(E)- | RT = 3.13 min, m/z = 385 |
| | | | | | ylidene]-heptyl | [M + H]⁺ |
| Ia-134 | S | H | Cl | H | 3,5,5-trimethyl-hexyl | RT = 2.84 min, m/z = 325 |
| | | | | | | [M + H]⁺ |
| Ia-135 | S | H | Cl | H | 3,3-dimethyl-butyl | RT = 2.22 min, m/z = 283 |
| | | | | | | [M + H]⁺ |
| Ia-136 | S | H | Cl | H | 3-methyl-butyl | RT = 2.03 min, m/z = 269 |
| | | | | | | [M + H]⁺ |
| Ia-137 | S | H | Cl | H | 2-(2,6,6-trimethyl- | RT = 2.92 min, m/z = 349 |
| | | | | | cyclohex-1-enyl)-ethyl | [M + H]⁺ |
| Ia-138 | S | H | Cl | H | (Z)-dec-4-enyl | RT = 3.03 min, m/z = 337 |
| | | | | | | [M + H]⁺ |
| Ia-139 | S | H | Cl | H | 3-methylsulfanyl- | RT = 1.80 min, m/z = 287 |
| | | | | | propyl | [M + H]⁺ |
| Ia-140 | S | H | Cl | H | 2-ethyl-hexyl | RT = 2.64 min, m/z = 311 |
| | | | | | | [M + H]⁺ |
| Ia-141 | S | Cl | H | H | 5,6-dihydro-2H-pyran- | RT = 1.69 min, m/z = 295 |
| | | | | | 3-yl-methyl | [M + H]⁺ |
| Ia-142 | S | Cl | H | H | furan-3-ylmethyl | RT = 1.76 min, m/z = 279 |
| | | | | | | [M + H]⁺ |
| Ia-143 | S | H | Cl | H | 5,6-dihydro-2H-pyran- | RT = 1.63 min, m/z = 295 |
| | | | | | 3-yl-methyl | [M + H]⁺ |
| Ia-144 | S | H | Cl | H | furan-3-ylmethyl | RT = 1.69 min, m/z = 279 |
| | | | | | | [M + H]⁺ |
| Ia-145 | S | Cl | H | H | 2,2-dimethyl-3-pyridin- | RT = 1.48 min, m/z = 346 |
| | | | | | 3-yl-propyl | [M + H]⁺ |
| Ia-146 | S | Cl | H | H | 3-methoxycarbonyl- | RT = 1.75 min, m/z = 299 |
| | | | | | propyl | [M + H]⁺ |
| Ia-147 | S | Cl | H | H | 2,6-dimethyl-hept-5- | RT = 2.79 min, m/z = 323 |
| | | | | | enyl | [M + H]⁺ |
| Ia-148 | S | Cl | H | H | 2-tert-butoxy-propyl | RT = 2.31 min, m/z = 313 |
| | | | | | | [M + H]⁺ |
| Ia-149 | S | H | Cl | H | 2-dimethylamino-2- | RT = 1.32 min, m/z = 312 |
| | | | | | methyl-propyl | [M + H]⁺ |
| Ia-150 | S | H | Cl | H | 2,2-dimethyl-3-pyridin- | RT = 1.45 min, m/z = 346 |
| | | | | | 3-yl-propyl | [M + H]⁺ |
| Ia-151 | S | H | Cl | H | 3-methoxycarbonyl- | RT = 1.68 min, m/z = 299 |
| | | | | | propyl | [M + H]⁺ |
| Ia-152 | S | H | Cl | H | 2,6-dimethyl-hept-5- | RT = 2.76 min, m/z = 323 |
| | | | | | enyl | [M + H]⁺ |
| Ia-153 | S | H | Cl | H | butyl | RT = 1.85 min, m/z = 255 |
| | | | | | | [M + H]⁺ |
| Ia-154 | S | H | Cl | H | 2-tert-butoxy-propyl | RT = 2.22 min, m/z = 313 |
| | | | | | | [M + H]⁺ |
| Ia-155 | S | H | Cl | H | 2-methyl-ethyl | RT = 1.51 min, m/z = 241 |
| | | | | | | [M + H]⁺ |
| Ia-156 | S | H | Cl | H | 2-phenyl-propyl | RT = 2.31 min, m/z = 317 |
| | | | | | | [M + H]⁺ |
| Ia-157 | S | H | Cl | H | 2,2-dimethyl-propyl | RT = 1.99 min, m/z = 269 |
| | | | | | | [M + H]⁺ |
| Ia-158 | S | H | Cl | H | 4-benzo[1,3]dioxol-5-yl- | RT = 2.49 min, m/z = 375 |
| | | | | | butyl | [M + H]⁺ |
| Ia-159 | S | H | Cl | H | 2-(4-fluoro-phenyl)- | RT = 2.91 min, m/z = 377 |
| | | | | | 4-methyl-pentyl | [M + H]⁺ |
| Ia-160 | S | H | Cl | H | 4,4-dimethyl-pentyl | RT = 2.51 min, m/z = 297 |
| | | | | | | [M + H]⁺ |
| Ia-161 | S | H | Cl | H | 3-phenyl-propyl | RT = 2.35 min, m/z = 317 |
| | | | | | | [M + H]⁺ |
| Ia-162 | S | H | Cl | H | propyl | RT = 1.62 min, m/z = 241 |
| | | | | | | [M + H]⁺ |
| Ia-163 | S | H | Cl | H | 2-(tetrahydro-pyran- | RT = 1.71 min, m/z = 311 |
| | | | | | 4-yl)-ethyl | [M + H]⁺ |
| Ia-164 | S | H | Cl | H | phenyl | RT = 3.00 min, m/z = 275 |
| | | | | | | [M + H]⁺ |
| Ia-165 | S | H | Cl | H | 9H-fluoren-9-yl | RT = 2.48 min, m/z = 363 |
| | | | | | | [M + H]⁺ |
| Ia-166 | S | H | Cl | H | 4-fluorophenyl | RT = 3.10 min, m/z = 293 |
| | | | | | | [M + H]⁺ |
| Ia-167 | S | H | Cl | H | 3-fluorophenyl | RT = 3.50 min, m/z = 293 |
| | | | | | | [M + H]⁺ |
| Ia-168 | S | H | Cl | H | 2-fluorophenyl | RT = 3.44 min, m/z = 293 |
| | | | | | | [M + H]⁺ |
| Ia-169 | S | H | Cl | H | 4-methoxyphenyl | RT = 2.23 min, m/z = 305 |
| | | | | | | [M + H]⁺ |
| Ia-170 | S | H | Cl | H | 3-methoxyphenyl | RT = 3.09 min, m/z = 305 |
| | | | | | | [M + H]⁺ |
| Ia-171 | S | H | Cl | H | 2-methoxyphenyl | RT = 2.18 min, m/z = 305 |
| | | | | | | [M + H]⁺ |
| Ia-172 | S | H | Cl | H | 4-trifluoromethyl- | RT = 3.78 min, m/z = 343 |
| | | | | | phenyl | [M + H]⁺ |
| Ia-173 | S | H | Cl | H | 3-trifluoromethyl- | RT = 3.83 min, m/z = 343 |
| | | | | | phenyl | [M + H]⁺ |
| Ia-174 | S | H | Cl | H | 2-trifluoromethyl- | RT = 3.76 min, m/z = 343 |
| | | | | | phenyl | [M + H]⁺ |
| Ia-175 | S | Cl | H | H | 2-phenyl-propyl | RT = 2.37 min, m/z = 317 |
| | | | | | | [M + H]⁺ |
| Ia-176 | S | Cl | H | H | 2,2-dimethyl-propyl | RT = 1.77 min, m/z = 268 |
| | | | | | | [M + H]⁺ |
| Ia-177 | S | Cl | H | H | 4-benzo[1,3]dioxol-5-yl- | RT = 2.50 min, m/z = 375 |
| | | | | | butyl | [M + H]⁺ |
| Ia-178 | S | Cl | H | H | 2-(4-fluoro-phenyl)- | RT = 2.97 min, m/z = 377 |
| | | | | | 4-methyl-pentyl | [M + H]⁺ |
| Ia-179 | S | Cl | H | H | 4,4-dimethyl-pentyl | RT = 2.55 min, m/z = 297 |
| | | | | | | [M + H]⁺ |
| Ia-180 | S | Cl | H | H | 3-phenyl-propyl | RT = 2.40 min, m/z = 317 |
| | | | | | | [M + H]⁺ |
| Ia-181 | S | Cl | H | H | 2-(tetrahydro-pyran- | RT = 1.77 min, m/z = 311 |
| | | | | | 4-yl)-ethyl | [M + H]+ |
| Ia-182 | S | Cl | H | H | (E)-3-(4-bromo- | RT = 2.57 min, m/z = 399 |
| | | | | | thiophen-2-yl)-allyl | [M + H]⁺ |
| Ia-183 | S | Cl | H | H | (E)-3-(3-methyl- | RT = 2.44 min, m/z = 335 |
| | | | | | thiophen-2-yl)-allyl | [M + H]⁺ |
| Ia-184 | S | Cl | H | H | 2-[1-furan-2-yl-meth- | RT = 2.58 min, m/z = 347 |
| | | | | | (E)-ylidene]-3-methyl-butyl | [M + H]⁺ |
| Ia-185 | S | Cl | H | H | (E)-2-methyl-3- | RT = 2.50 min, m/z = 329 |
| | | | | | phenyl-allyl | [M + H]⁺ |
| Ia-186 | S | H | Cl | H | (E)-3-furan-2-yl- | RT = 2.07 min, m/z = 305 |
| | | | | | allyl | [M + H]⁺ |
| Ia-187 | S | H | Cl | H | (E)-3-(3,5-dichloro- | RT = 2.80 min, m/z = 385 |
| | | | | | phenyl)-allyl | [M + H]+ |
| Ia-188 | S | H | Cl | H | (E)-3-(4-bromo- | RT = 2.52 min, m/z = 401 |
| | | | | | thiophen-2-yl)-allyl | [M + H]⁺ |
| Ia-189 | S | H | Cl | H | (E)-3-(3-methyl- | RT = 2.38 min, m/z = 335 |
| | | | | | thiophen-2-yl)-allyl | [M + H]⁺ |
| Ia-190 | S | H | Cl | H | 2-[1-furan-2-yl-meth- | RT = 2.20 min, m/z = 319 |
| | | | | | (E)-ylidene]-propyl | [M + H]+ |
| Ia-191 | S | H | Cl | H | 2-[1-furan-2-yl-meth- | RT = 2.52 min, m/z = 347 |
| | | | | | (E)-ylidene]-3-methyl-butyl | [M + H]⁺ |
| Ia-192 | S | H | Cl | H | (E)-2-methyl-3- | RT = 2.44 min, m/z = 329 |
| | | | | | phenyl-allyl | [M + H]⁺ |
| Ia-193 | S | H | Cl | H | 5-hydroxypentyl | RT = 1.50 min, m/z = 285 |
| | | | | | | [M + H]+ |
| Ia-194 | S | H | Cl | H | tetrahydro-thiopyran- | RT = 2.03 min, m/z = 313 |
| | | | | | 3-ylmethyl | [M + H]⁺ |
| Ia-195 | S | H | Cl | H | 1H-Indol-3-ylmethyl | RT = 2.07 min, m/z = 328 |
| | | | | | | [M + H]⁺ |
| Ia-196 | S | H | Cl | H | 3-dimethylaminomethyl- | RT = 1.42 min, m/z = 340 |
| | | | | | tetrahydro-furan-3- | [M + H]⁺ |
| | | | | | ylmethyl | |
| Ia-197 | S | H | Cl | H | 5-methyl-1H-imidazol- | RT = 1.23 min, m/z = 293 |
| | | | | | 4-ylmethyl | [M + H]+ |
| Ia-198 | S | H | Cl | H | tetrahydro-pyran- | RT = 1.70 min, m/z = 297 |
| | | | | | 3-ylmethyl | [M + H]⁺ |
| Ia-199 | S | H | Cl | H | 1-(4-chloro-phenyl)-1H- | RT = 2.70 min, m/z = 397 |
| | | | | | pyrrol-3-ylmethyl | [M + H]⁺ |
| Ia-200 | S | H | Cl | H | 2,5-dihydro-thiophen- | RT = 1.86 min, m/z = 297 |
| | | | | | 3-ylmethyl | [M + H]⁺ |
| Ia-201 | S | H | Cl | H | pyridin-3-ylmethyl | RT = 1.28 min, m/z = 290 |
| | | | | | | [M + H]⁺ |
| Ia-202 | S | H | Cl | H | 1-tert-Butyl-1H-pyrazol- | RT = 2.01 min, m/z = 335 |
| | | | | | 4-ylmethyl | [M + H]⁺ |
| Ia-203 | S | H | Cl | H | 3-isobutyl-isoxazol-5- | RT = 2.25 min, m/z = 336 |
| | | | | | ylmethyl | [M + H]⁺ |
| Ia-204 | S | H | Cl | H | 2-chloro-pyridin-5- | RT = 1.82 min, m/z = 324 |
| | | | | | ylmethyl | [M + H]⁺ |
| Ia-205 | S | H | Cl | H | 3-chloro-1H-indol-2- | RT = 2.51 min, m/z = 362 |
| | | | | | ylmethyl | [M + H]⁺ |
| Ia-206 | S | H | Cl | H | furan-2-ylmethyl | RT = 1.72 min, m/z = 279 |
| | | | | | | [M + H]⁺ |
| Ia-207 | S | H | Cl | H | 1-(4-chloro-phenyl)-5- | RT = 2.78 min, m/z = 457 |
| | | | | | trifluoromethyl-1H- | [M + H]⁺ |
| | | | | | pyrazol-4-ylmethyl | |
| Ia-208 | S | H | Cl | H | 3-tert-butyl-isoxazol- | RT = 2.22 min, m/z = 336 |
| | | | | | 5-ylmethyl | [M + H]⁺ |
| Ia-209 | S | H | Cl | H | 2-dimethylamino-thiazol- | RT = 1.41 min, m/z = 339 |
| | | | | | 5-ylmethyl | [M + H]⁺ |
| Ia-210 | S | H | Cl | H | pyridin-4-ylmethyl | RT = 1.21 min, m/z = 290 |
| | | | | | | [M + H]⁺ |
| Ia-211 | S | H | Cl | H | 3-isopropyl-isoxazol-5- | RT = 2.03 min, m/z = 322 |
| | | | | | ylmethyl | [M + H]⁺ |
| Ia-212 | S | H | Cl | H | 4-cyano-thiophen-2- | RT = 1.75 min, m/z = 320 |
| | | | | | ylmethyl | [M + H]⁺ |
| Ia-213 | S | H | Cl | H | 4-bromo-thiophen-2- | RT = 2.15 min, m/z = 374 |
| | | | | | ylmethyl | [M + H]⁺ |
| Ia-214 | S | H | Cl | H | pyridin-2-ylmethyl | RT = 1.48 min, m/z = 290 |
| | | | | | | [M + H]⁺ |
| Ia-215 | S | H | Cl | H | thiophen-2-ylmethyl | RT = 1.84 min, m/z = 295 |
| | | | | | | [M + H]⁺ |
| Ia-216 | S | H | Cl | H | 1-phenyl-propyl | RT = 2.24 min, m/z = 317 |
| | | | | | | [M + H] |
| | | | | | | RT = 2.31 min, m/z = 317 |
| | | | | | | [M + H] |
| Ia-217 | S | H | Cl | H | 1-methyl-prop-2-ynyl | RT = 1.56 min, m/z = 251 |
| | | | | | | [M + H] |
| Ia-218 | S | H | Cl | H | 2-hexyl | RT = 2.18 min, m/z = 283 |
| | | | | | | [M + H] |
| Ia-219 | S | H | Cl | H | 3-pentyl | RT = 1.86 min, m/z = 269 |
| | | | | | | [M + H] |
| Ia-220 | S | H | Cl | H | 2-octyl | RT = 2.66 min, m/z = 311 |
| | | | | | | [M + H] |
| Ia-221 | S | H | Cl | H | 1-methyl-tridecyl | RT = 3.91 min, m/z = 396 |
| | | | | | | [M + H] |
| Ia-222 | S | H | H | H | sec-butyl | RT = 1.69 min, m/z = 255 |
| | | | | | | [M + H] |
| Ia-223 | S | H | Cl | H | (E)-3-(4-chloro-phenyl)- | RT = 2.52 min, m/z = 349 |
| | | | | | allyl | [M + H] |
| Ia-224 | S | Cl | Cl | H | (E)-3-(4-chloro-phenyl)- | RT = 2.82 min, m/z = 383 |
| | | | | | allyl | [M + H] |
| Ia-225 | S | H | Br | H | 3-methyl-benzyl | RT = 1.42 min, m/z = 317 |
| | | | | | | [M + H] |
| Ia-226 | S | H | CC-Ph | H | 2,4-dichlorobenzyl | RT = 2.89 min, m/z = 423 |
| | | | | | | [M + H] |
| Ia-227 | S | H | CC-Ph | H | 3-methyl-benzyl | RT = 2.74 min, m/z = 369 |
| | | | | | | [M + H] |
| Ia-228 | S | H | CC—CH₂OCH₃ | H | 3-methyl-benzyl | RT = 2.06 min, m/z = 337 |
| | | | | | | [M + H] |
| Ia-229 | S | Cl | Cl | H | 2-chloro-pyridin- | RT = 3.77 min, m/z = 305 |
| | | | | | 4-ylmethyl | [M + H] |
| Ia-230 | S/R | Cl | H | H | 2,4-dichlorobenzyl | RT = 2.38 min, m/z = 359 |
| | | | | | | [M + H] |
| Ia-231 | S | H | CH═CH₂ | H | 2,4-dichlorobenzyl | RT = 1.87 min, m/z = 349 |
| | | | | | | [M + H] |
| Ia-232 | S | Cl | Cl | H | 1-methyl-prop-2-ynyl | RT = 2.02 min, m/z = 285 |
| | | | | | | [M + H] |
| Ia-233 | — | Cl | Cl | H | 3-amino-4-(5,6- | |
| | | | | | dichloro-pyridin-3- | |
| | | | | | yloxy)-butyl | |
| Ia-234 | S | H | Cl | H | 4-(5-chloropyridin-3- | |
| | | | | | yloxy)-3-(4-methyl- | |
| | | | | | benzoylamino)-butyl | |
| Ia-235 | S | H | Cl | H | 3-(3-chloro- | |
| | | | | | benzoylamino)-4-(5- | |
| | | | | | chloro-pyridin-3-yloxy)- | |
| | | | | | butyl | |
| Ia-236 | S | H | Cl | H | 3-benzoylamino-4-(5- | |
| | | | | | chloro-pyridin-3-yloxy)- | |
| | | | | | butyl | |
| Ia-237 | S | H | Cl | H | 4-(5-chloropyridin-3- | |
| | | | | | yloxy)-3-(4- | |
| | | | | | trifluoromethyl- | |
| | | | | | benzoylamino)-butyl | |
| Ia-238 | S | H | Cl | H | 3-(4-chloro- | |
| | | | | | benzoylamino)-4-(5- | |
| | | | | | chloro-pyridin-3-yloxy)- | |
| | | | | | butyl | |
| Ia-239 | S | H | Cl | H | 3-(4-nitro- | |
| | | | | | benzoylamino)-4-(5- | |
| | | | | | chloro-pyridin-3-yloxy)- | |
| | | | | | butyl | |
| Ia-240 | S | H | Cl | H | 4-(5-chloropyridin-3- | |
| | | | | | yloxy)-3-(3- | |
| | | | | | trifluoromethyl- | |
| | | | | | benzoylamino)-butyl | |
| Ia-241 | S | Cl | Cl | H | 4-(5,6-dichloropyridin- | |
| | | | | | 3-yloxy)-3-(4-methyl- | |
| | | | | | benzoylamino)-butyl | |
| Ia-242 | S | Cl | Cl | H | 3-(3-chloro- | |
| | | | | | benzoylamino)-4-(5,6- | |
| | | | | | dichloro-pyridin-3- | |
| | | | | | yloxy)-butyl | |
| Ia-243 | S | Cl | Cl | H | 3-(4-methoxy- | |
| | | | | | benzoylamino)-4-(5,6- | |
| | | | | | dichloro-pyridin-3- | |
| | | | | | yloxy)-butyl | |
| Ia-244 | S | Cl | Cl | H | 3-(4-trifluoromethyl- | |
| | | | | | benzoylamino)-4-(5,6- | |
| | | | | | dichloro-pyridin-3- | |
| | | | | | yloxy)-butyl | |
| Ia-245 | S | Cl | Cl | H | 3-(4-chloro- | |
| | | | | | benzoylamino)-4-(5,6- | |
| | | | | | dichloro-pyridin-3- | |
| | | | | | yloxy)-butyl | |
| Ia-246 | S | Cl | Cl | H | 3-(4-nitro- | |
| | | | | | benzoylamino)-4-(5,6- | |
| | | | | | dichloro-pyridin-3- | |
| | | | | | yloxy)-butyl | |
| Ia-247 | S | Cl | Cl | H | 3-(3-trifluoromethyl- | |
| | | | | | benzoylamino)-4-(5,6- | |
| | | | | | dichloro-pyridin-3- | |
| | | | | | yloxy)-butyl | |
| Ia-248 | S | Cl | H | H | 4-(6-chloropyridin-3- | |
| | | | | | yloxy)-3-(4-methyl- | |
| | | | | | benzoylamino)-butyl | |
| Ia-249 | S | Cl | H | H | 3-(2-chloro- | |
| | | | | | benzoylamino)-4-(6- | |
| | | | | | chloro-pyridin-3-yloxy)- | |
| | | | | | butyl | |
| Ia-250 | S | Cl | H | H | 3-(4-methoxy- | |
| | | | | | benzoylamino)-4-(6- | |
| | | | | | chloro-pyridin-3-yloxy)- | |
| | | | | | butyl | |
| Ia-251 | S | Cl | H | H | 3-(pyridine-3-carbonyl- | |
| | | | | | amino)-4-(6-chloro- | |
| | | | | | pyridin-3-yloxy)-butyl | |
| Ia-252 | S | Cl | H | H | 3-(4-trifluoromethyl- | |
| | | | | | benzoylamino)-4-(6- | |
| | | | | | chloropyridin-3-yloxy)- | |
| | | | | | butyl | |
| Ia-253 | S | Cl | H | H | 3-(4-chloro- | |
| | | | | | benzoylamino)-4-(6- | |
| | | | | | chloropyridin-3-yloxy)- | |
| | | | | | butyl | |
| Ia-254 | S | Cl | H | H | 3-(4-nitro- | |
| | | | | | benzoylamino)-4-(6- | |
| | | | | | chloropyridin-3-yloxy)- | |
| | | | | | butyl | |
| Ia-255 | S | Cl | H | H | 3-(3-trifluoromethyl- | |
| | | | | | benzoylamino)-4-(6- | |
| | | | | | chloropyridin-3-yloxy)- | |
| | | | | | butyl | |
| Ia-256 | S | H | Br | H | 3-(4-methyl- | |
| | | | | | benzoylamino)-4-(5- | |
| | | | | | bromopyridin-3-yloxy)- | |
| | | | | | butyl | |
| Ia-257 | S | H | Br | H | 3-(2-chloro- | |
| | | | | | benzoylamino)-4-(5- | |
| | | | | | bromopyridin-3-yloxy)- | |
| | | | | | butyl | |
| Ia-258 | S | H | Br | H | 3-(4-methoxy- | |
| | | | | | benzoylamino)-4-(5- | |
| | | | | | bromopyridin-3-yloxy)- | |
| | | | | | butyl | |
| Ia-259 | S | H | Br | H | 3-(4-trifluoromethyl- | |
| | | | | | benzoylamino)-4-(5- | |
| | | | | | bromopyridin-3-yloxy)- | |
| | | | | | butyl | |
| Ia-260 | S | H | Br | H | 3-(4-chloro- | |
| | | | | | benzoylamino)-4-(5- | |
| | | | | | bromopyridin-3-yloxy)- | |
| | | | | | butyl | |
| Ia-261 | S | H | Br | H | 3-(4-nitro- | |
| | | | | | benzoylamino)-4-(5- | |
| | | | | | bromopyridin-3-yloxy)- | |
| | | | | | butyl | |
| Ia-262 | — | H | Cl | CH₃ | 3-(4-methyl- | |
| | | | | | benzoylamino)-4-(5- | |
| | | | | | chloro-pyridin-3-yloxy)- | |
| | | | | | pentyl | |
| Ia-263 | — | H | Cl | CH₃ | 3-(3-chloro- | |
| | | | | | benzoylamino)-4-(5- | |
| | | | | | chloro-pyridin-3-yloxy)- | |
| | | | | | pentyl | |
| Ia-264 | — | H | Cl | CH₃ | 3-(4-trifluoromethyl- | |
| | | | | | benzoylamino)-4-(5- | |
| | | | | | chloro-pyridin-3-yloxy)- | |
| | | | | | pentyl | |
| Ia-265 | — | H | Cl | CH₃ | 3-(4-chloro- | |
| | | | | | benzoylamino)-4-(5- | |
| | | | | | chloro-pyridin-3-yloxy)- | |
| | | | | | pentyl | |
| Ia-266 | — | H | Cl | CH₃ | 3-(4-nitro- | |
| | | | | | benzoylamino)-4-(5- | |
| | | | | | chloro-pyridin-3-yloxy)- | |
| | | | | | pentyl | |
| Ia-267 | — | H | Cl | CH₃ | 3-(3-trifluoromethyl- | |
| | | | | | benzoylamino)-4-(5- | |
| | | | | | chloro-pyridin-3-yloxy)- | |
| | | | | | pentyl | |
| Ia-268 | S | H | Cl | H | (cyanomethyl- | |
| | | | | | carbamoyl)-phenyl- | |
| | | | | | methyl | |
| Ia-269 | S | H | Cl | H | 3-(3-methyl- | |
| | | | | | benzoylamino)-4-(5- | |
| | | | | | chloropyridin-3-yloxy)- | |
| | | | | | butyl | |
| Ia-270 | S | H | Cl | H | 3-(2-trifluoromethyl- | |
| | | | | | benzoylamino)-4-(5- | |
| | | | | | chloropyridin-3-yloxy)- | |
| | | | | | butyl | |
| Ia-271 | S | H | Cl | H | 3-(2-methyl- | |
| | | | | | benzoylamino)-4-(5- | |
| | | | | | chloropyridin-3-yloxy)- | |
| | | | | | butyl | |
| Ia-272 | S | H | Cl | H | 3-(2,6-dichloro- | |
| | | | | | benzoylamino)-4-(5- | |
| | | | | | chloropyridin-3-yloxy)- | |
| | | | | | butyl | |
| Ia-273 | S | H | Cl | H | 3-(2-chloro- | |
| | | | | | benzoylamino)-4-(5- | |
| | | | | | chloropyridin-3-yloxy)- | |
| | | | | | butyl | |
| Ia-274 | S | H | Cl | H | 3-(3-fluoro- | |
| | | | | | benzoylamino)-4-(5- | |
| | | | | | chloropyridin-3-yloxy)- | |
| | | | | | butyl | |
| Ia-275 | S | Cl | Cl | H | 3-(3-methyl- | |
| | | | | | benzoylamino)-4-(5,6- | |
| | | | | | dichloropyridin-3- | |
| | | | | | yloxy)-butyl | |
| Ia-276 | S | Cl | Cl | H | 3-(2-trifluoromethyl- | |
| | | | | | benzoylamino)-4-(5,6- | |
| | | | | | dichloropyridin-3- | |
| | | | | | yloxy)-butyl | |
| Ia-277 | S | Cl | Cl | H | 3-(2-methyl- | |
| | | | | | benzoylamino)-4-(5,6- | |
| | | | | | dichloropyridin-3- | |
| | | | | | yloxy)-butyl | |
| Ia-278 | S | Cl | Cl | H | 3-(2,6-dichloro- | |
| | | | | | benzoylamino)-4-(5,6- | |
| | | | | | dichloropyridin-3- | |
| | | | | | yloxy)-butyl | |
| Ia-279 | S | Cl | Cl | H | 3-(2-chloro- | |
| | | | | | benzoylamino)-4-(5,6- | |
| | | | | | dichloropyridin-3- | |
| | | | | | yloxy)-butyl | |
| Ia-280 | S | Cl | Cl | H | 3-(3-fluoro- | |
| | | | | | benzoylamino)-4-(5,6- | |
| | | | | | dichloropyridin-3- | |
| | | | | | yloxy)-butyl | |
| Ia-281 | S | H | Br | H | 3-(3-nitro- | |
| | | | | | benzoylamino)-4-(5- | |
| | | | | | bromopyridin-3-yloxy)- | |
| | | | | | butyl | |
| Ia-282 | S | H | Br | H | 3-(3-methyl- | |
| | | | | | benzoylamino)-4-(5- | |
| | | | | | bromopyridin-3-yloxy)- | |
| | | | | | butyl | |
| Ia-283 | S | H | Br | H | 3-(2-trifluoromethyl- | |
| | | | | | benzoylamino)-4-(5- | |
| | | | | | bromopyridin-3-yloxy)- | |
| | | | | | butyl | |
| Ia-284 | S | H | Br | H | 3-(2-methyl- | |
| | | | | | benzoylamino)-4-(5- | |
| | | | | | bromopyridin-3-yloxy)- | |
| | | | | | butyl | |
| Ia-285 | S | H | Br | H | 3-(2,6-dichloro- | |
| | | | | | benzoylamino)-4-(5- | |
| | | | | | bromopyridin-3-yloxy)- | |
| | | | | | butyl | |
| Ia-286 | S | H | Br | H | 3-(3-fluoro- | |
| | | | | | benzoylamino)-4-(5- | |
| | | | | | bromopyridin-3-yloxy)- | |
| | | | | | butyl | |
| Ia-287 | S | Cl | H | H | 3-(3-nitro- | |
| | | | | | benzoylamino)-4-(6- | |
| | | | | | chloropyridin-3-yloxy)- | |
| | | | | | butyl | |
| Ia-288 | S | Cl | H | H | 3-(3-methyl- | |
| | | | | | benzoylamino)-4-(6- | |
| | | | | | chloropyridin-3-yloxy)- | |
| | | | | | butyl | |
| Ia-289 | S | Cl | H | H | 3-(2-trifluoromethyl- | |
| | | | | | benzoylamino)-4-(6- | |
| | | | | | chloropyridin-3-yloxy)- | |
| | | | | | butyl | |
| Ia-290 | S | Cl | H | H | 3-(2-methyl- | |
| | | | | | benzoylamino)-4-(6- | |
| | | | | | chloropyridin-3-yloxy)- | |
| | | | | | butyl | |
| Ia-291 | S | Cl | H | H | 3-(2,6-dichloro- | |
| | | | | | benzoylamino)-4-(6- | |
| | | | | | chloropyridin-3-yloxy)- | |
| | | | | | butyl | |
| Ia-292 | S | Cl | H | H | 3-(2-chloro- | |
| | | | | | benzoylamino)-4-(6- | |
| | | | | | chloropyridin-3-yloxy)- | |
| | | | | | butyl | |
| Ia-293 | S | Cl | H | H | 3-(3-fluoro- | |
| | | | | | benzoylamino)-4-(6- | |
| | | | | | chloropyridin-3-yloxy)- | |
| | | | | | butyl | |
| Ia-294 | S | H | Cl | H | 3-(2-fluoro- | |
| | | | | | benzoylamino)-4-(5- | |
| | | | | | chloropyridin-3-yloxy)- | |
| | | | | | butyl | |
| Ia-295 | S | H | Cl | H | 3-(3-methoxy- | |
| | | | | | benzoylamino)-4-(5- | |
| | | | | | chloropyridin-3-yloxy)- | |
| | | | | | butyl | |
| Ia-296 | S | Cl | Cl | H | 3-(2-fluoro- | |
| | | | | | benzoylamino)-4-(5,6- | |
| | | | | | dichloropyridin-3- | |
| | | | | | yloxy)-butyl | |
| Ia-297 | S | Cl | Cl | H | 3-(3-methoxy- | |
| | | | | | benzoylamino)-4-(5,6- | |
| | | | | | dichloropyridin-3- | |
| | | | | | yloxy)-butyl | |
| Ia-298 | S | Cl | Cl | H | 3-(thiophene-3- | |
| | | | | | carbonyl)-amino)-4- | |
| | | | | | (5,6-dichloropyridin-3- | |
| | | | | | yloxy)-butyl | |
| Ia-299 | S | H | Br | H | 3-(2-fluoro- | |
| | | | | | benzoylamino)-4-(5- | |
| | | | | | bromopyridin-3-yloxy)- | |
| | | | | | butyl | |
| Ia-300 | S | H | Br | H | 3-(3-methoxy- | |
| | | | | | benzoylamino)-4-(5- | |
| | | | | | bromopyridin-3-yloxy)- | |
| | | | | | butyl | |
| Ia-301 | S | H | Br | H | 3-(thiophene-2- | |
| | | | | | carbonyl)-amino)-4-(5- | |
| | | | | | bromopyridin-3-yloxy)- | |
| | | | | | butyl | |
| Ia-302 | S | H | Br | H | 3-(pyridin-2-carbonyl)- | |
| | | | | | amino)-4-(5- | |
| | | | | | bromopyridin-3-yloxy)- | |
| | | | | | butyl | |
| Ia-303 | S | Cl | H | H | 3-(2-fluoro- | |
| | | | | | benzoylamino)-4-(6- | |
| | | | | | chloropyridin-3-yloxy)- | |
| | | | | | butyl | |
| Ia-304 | S | Cl | H | H | 3-(3-methoxy- | |
| | | | | | benzoylamino)-4-(6- | |
| | | | | | chloropyridin-3-yloxy)- | |
| | | | | | butyl | |
| Ia-305 | S | Cl | H | H | 3-(pyridin-2-carbonyl)- | |
| | | | | | amino)-4-(5- | |
| | | | | | bromopyridin-3-yloxy)- | |
| | | | | | butyl | |
| Ia-306 | — | H | Cl | H | 3-(4-methoxy- | |
| | | | | | benzoylamino)-4-(5- | |
| | | | | | chloropyridin-3-yloxy)- | |
| | | | | | butyl | |
| Ia-307 | — | H | Br | H | 3-(3-trifluoromethyl- | |
| | | | | | benzoylamino)-4-(5- | |
| | | | | | bromopyridin-3-yloxy)- | |
| | | | | | butyl | |
| Ia-308 | — | Cl | H | H | 3-(thiophen-2- | |
| | | | | | carbonyl)-amino)- | |
| | | | | | propyl | |

Ex = example
Ph = phenyl
CC = ethynyl

TABLE II
Compounds of the formula Ib (= compounds of the formula I
with A being CH₂, n = 0, y = 0 and R³ = H.

| | |
|---|---|
| | (Ib-S) |
| 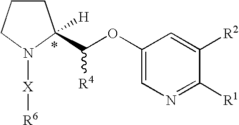 | |
| | (Ib-R) |
| 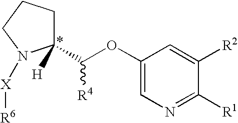 | |

| Ex. | C* | R¹ | R² | R⁴ | X—R⁶ | Physical Data |
|---|---|---|---|---|---|---|
| Ib-1 | R | H | Cl | H | 2-hydroxy-butyl | RT = 1.64 min, |
| | | | | | | m/z = 285 [M + H]+ |
| Ib-2 | S | Cl | H | H | 2-hydroxy-butyl | RT = 1.70 min, |
| | | | | | | m/z = 285 [M + H]+ |
| Ib-3 | R | H | Cl | H | 1-methyl-ethyl | RT = 1.63 min, |
| | | | | | | m/z = 255 [M + H]+ |
| Ib-4 | R | H | Cl | H | 2-acetoxy-butyl | RT = 1.98 min, |
| | | | | | | m/z = 327 [M + H]+ |
| Ib-5 | R | H | Cl | H | 2,3-diacetoxy-propyl | RT = 1.88 min, |
| | | | | | | m/z = 371 [M + H]+ |
| Ib-6 | R | H | Cl | H | 5,6-dihydro-2H-pyran-3-yl- | RT = 1.69 min, |
| | | | | | methyl | m/z = 309 [M + H]+ |
| Ib-7 | R | H | Cl | H | 1-furan-3-ylmethyl | RT = 1.78 min, |
| | | | | | | m/z = 293 [M + H]+ |
| Ib-8 | S | Cl | H | H | 5,6-dihydro-2H-pyran-3-yl- | RT = 1.75 min, |
| | | | | | methyl | m/z = 309 [M + H]+ |
| Ib-9 | S | Cl | H | H | 1-furan-3-ylmethyl | RT = 1.85 min, |
| | | | | | | m/z = 293 [M + H]+ |
| Ib-10 | S | H | Cl | H | 5,6-dihydro-2H-pyran-3-yl- | RT = 1.71 min, |
| | | | | | methyl | m/z = 309 [M + H]+ |
| Ib-11 | S | H | Cl | H | 1-furan-3-ylmethyl | RT = 1.79 min, |
| | | | | | | m/z = 293 [M + H]+ |
| Ib-12 | S | H | H | CH₃ | 5,6-dihydro-2H-pyran-3-yl- | RT = 0.84 min, |
| | | | | | methyl | m/z = 289 [M + H]+ |
| | | | | | | RT = 0.92 min, |
| | | | | | | m/z = 289 [M + H]+ |
| Ib-13 | S | H | H | CH₃ | 1-furan-3-ylmethyl | RT = 0.93 min, |
| | | | | | | m/z = 273 [M + H]+ |
| Ib-14 | S | Cl | H | H | 5,6-dihydro-2H-pyran-3-yl- | RT = 1.69 min, |
| | | | | | methyl | m/z = 295 [M + H]+ |
| Ib-15 | S | Cl | H | H | 1-furan-3-ylmethyl | RT = 1.76 min, |
| | | | | | | m/z = 279 [M + H]+ |
| Ib-16 | S | H | Cl | H | 5,6-dihydro-2H-pyran-3-yl- | RT = 1.63 min, |
| | | | | | methyl | m/z = 295 [M + H]+ |
| Ib-17 | S | H | Cl | H | 5,6-dihydro-2H-pyran-3-yl- | RT = 1.71 min, |
| | | | | | methyl | m/z = 309 [M + H]+ |
| Ib-18 | S | H | Cl | H | 1-furan-3-ylmethyl | RT = 1.79 min, |
| | | | | | | m/z = 293 [M + H]+ |
| Ib-19 | S | H | H | CH₃ | 1-methyl-1H-pyrrol-2-ylmethyl | RT = 1.21 min, |
| | | | | | | m/z = 286 [M + H]+ |
| Ib-20 | S | H | H | CH₃ | 1H-indol-3-ylmethyl | RT = 1.45 min, |
| | | | | | | m/z = 322 [M + H]+ |
| Ib-21 | S | H | H | CH₃ | 1-(4-chloro-phenyl)-1H-pyrrol- | RT = 2.10 min, |
| | | | | | 3-ylmethyl | m/z = 382 [M + H]+ |
| Ib-22 | S | H | H | CH₃ | 2,5-dihydro-thiophen-3- | RT = 1.01 min, |
| | | | | | ylmethyl | m/z = 291 [M + H]+ |
| | | | | | (2 diastereomers) | RT = 1.12 min, |
| | | | | | | m/z = 291 [M + H]+ |
| Ib-23 | S | H | H | CH₃ | thiophen-2-ylmethyl | RT = 1.18 min, |
| | | | | | (2 diastereomers) | m/z = 289 [M + H]+ |
| | | | | | | RT = 1.31 min, |
| | | | | | | m/z = 289 [M + H]+ |
| Ib-24 | S | H | H | CH₃ | 1-tert-butyl-1H-pyrazol-4- | RT = 1.46 min, |
| | | | | | ylmethyl (2 diastereomers) | m/z = 329 [M + H]+ |
| | | | | | | RT = 1.53 min, |
| | | | | | | m/z = 329 [M + H]+ |
| Ib-25 | S | H | H | CH₃ | 3-isobutyl-isoxazol-5-ylmethyl | RT = 1.67 min, |
| | | | | | | m/z = 330 [M + H]+ |
| | | | | | | RT = 1.74 min, |
| | | | | | | m/z = 330 [M + H]+ |
| Ib-26 | S | H | H | CH₃ | 6-chloro-pyridin-3-ylmethyl | RT = 1.14 min, |
| | | | | | | m/z = 318 [M + H]+ |
| Ib-27 | S | H | H | CH₃ | 3-chloro-1H-indol-2-ylmethyl | RT = 1.87 min, |
| | | | | | | m/z = 356 [M + H]+ |
| Ib-28 | S | H | H | CH₃ | furan-2-ylmethyl | RT = 0.92 min, |
| | | | | | (2 diastereomers) | m/z = 273 [M + H]+ |
| | | | | | | RT = 1.10 min, |
| | | | | | | m/z = 273 [M + H]+ |
| Ib-29 | S | Cl | H | H | tetrahydro-thiopyran-3- | RT = 2.12 min, |
| | | | | | ylmethyl | m/z = 327 [M + H]+ |
| Ib-30 | S | Cl | H | H | 1-methyl-1H-pyrrol-2-ylmethyl | RT = 1.99 min, |
| | | | | | | m/z = 306 [M + H]+ |
| Ib-31 | S | Cl | H | H | 1H-indol-3-ylmethyl | RT = 2.21 min, |
| | | | | | | m/z = 342 [M + H]+ |
| Ib-32 | S | Cl | H | H | 3-dimethylaminomethyl- | RT = 1.48 min, |
| | | | | | tetrahydrofuran-3-ylmethyl | m/z = 354 [M + H]+ |
| Ib-33 | S | Cl | H | H | 5-methyl-1H-imidazol-4- | RT = 1.35 min, |
| | | | | | ylmethyl | m/z = 307 [M + H]+ |
| Ib-34 | S | Cl | H | H | tetrahydro-pyran-3-ylmethyl | RT = 1.80 min, |
| | | | | | | m/z = 311 [M + H]+ |
| Ib-35 | S | Cl | H | H | 1-(4-chloro-phenyl)-1H-pyrrol- | RT = 2.80 min, |
| | | | | | ylmethyl | m/z = 402 [M + H]+ |
| Ib-36 | S | Cl | H | H | 2,5-dihydro-thiophen-3- | RT = 1.96 min, |
| | | | | | ylmethyl | m/z = 311 [M + H]+ |
| Ib-37 | S | Cl | H | H | thiophen-2-ylmethyl | RT = 1.97 min, |
| | | | | | | m/z = 309 [M + H]+ |
| Ib-38 | S | Cl | H | H | 1-tert-butyl-1H-pyrazol-4- | RT = 2.11 min, |
| | | | | | ylmethyl | m/z = 349 [M + H]+ |
| Ib-39 | S | Cl | H | H | 3-isobutyl-isoxazol-5-ylmethyl | RT = 2.36 min, |
| | | | | | | m/z = 350 [M + H]+ |
| Ib-40 | S | Cl | H | H | 6-chloro-pyridin-3-ylmethyl | RT = 1.90 min, |
| | | | | | | m/z = 388 [M + H]+ |
| Ib-41 | S | Cl | H | H | 3-chloro-1H-indol-2-ylmethyl | RT = 2.61 min, |
| | | | | | | m/z = 376 [M + H]+ |
| Ib-42 | S | Cl | H | H | furan-2-ylmethyl | RT = 1.87 min, |
| | | | | | | m/z = 293 [M + H]+ |
| Ib-43 | S | H | Cl | H | 1-methyl-ethyl | RT = 1.62 min, |
| | | | | | | m/z = 255 [M + H]+ |
| Ib-44 | S | Cl | Cl | H | 3,5-dichloro-benzyl | |
| Ib-45 | R | H | Cl | H | 3,5-dichloro-benzyl | |
| Ib-46 | S | Cl | Cl | H | cyclopropyl-methyl | |
| Ib-47 | S | Cl | Cl | H | phenethyl | |
| Ib-48 | S | Cl | Cl | H | benzyl | |
| Ib-49 | S | Cl | Cl | H | 4-methoxy-benzyl | |
| Ib-50 | S | Cl | Cl | H | 4-chloro-benzyl | |
| Ib-51 | R | H | Cl | H | phenethyl | |
| Ib-52 | R | H | Cl | H | cyclopropyl-methyl | |
| Ib-53 | R | H | Cl | H | allyl | |
| Ib-54 | S | Cl | Cl | H | ethyl | |
| Ib-55 | R | H | Cl | H | benzyl | |
| Ib-56 | R | H | Cl | H | 4-methoxy-benzyl | |
| Ib-57 | R | H | Cl | H | 4-chloro-benzyl | |
| Ib-58 | R | H | Cl | H | 2-chloro-ethyl | |
| Ib-59 | R | H | Cl | H | 3-methylsulfanyl-propyl | |
| Ib-60 | R | H | Cl | H | butyl | |
| Ib-61 | R | H | Cl | H | pyridin-2-ylmethyl | |
| Ib-62 | R | H | Cl | H | pyridin-3-ylmethyl | |
| Ib-63 | R | H | Cl | H | pyridin-4-ylmethyl | |
| Ib-64 | R | H | Cl | H | tetrahydro-pyran-4-ylmethyl | |
| Ib-65 | R | H | Cl | H | 2-chloro-thiazol-5-ylmethyl | |
| Ib-66 | R | Cl | H | H | 2-methyl-propyl | |
| Ib-67 | R | Cl | H | H | cyclopropyl-methyl | |
| Ib-68 | R | Cl | H | H | phenethyl | |
| Ib-69 | R | Cl | H | H | benzyl | |
| Ib-70 | R | Cl | H | H | 4-methoxy-benzyl | |
| Ib-71 | R | Cl | H | H | 4-chloro-benzyl | |
| Ib-72 | R | Cl | H | H | 2-chloro-ethyl | |
| Ib-73 | R | Cl | H | H | 3-methylsulfanyl-propyl | |
| Ib-74 | R | Cl | H | H | butyl | |
| Ib-75 | R | Cl | H | H | 3,5-dichloro-benzyl | |
| Ib-76 | R | Cl | H | H | pyridin-2-ylmethyl | |
| Ib-77 | R | Cl | H | H | pyridin-3-ylmethyl | |
| Ib-78 | R | Cl | H | H | pyridin-4-ylmethyl | |
| Ib-79 | R | Cl | H | H | tetrahydro-pyran-4-ylmethyl | |
| Ib-80 | R | Cl | H | H | 2-chloro-thiazol-5-ylmethyl | |
| Ib-81 | S | Cl | H | H | 2-methyl-propyl | |
| Ib-82 | S | Cl | H | H | cyclopropyl-methyl | |
| Ib-83 | S | Cl | H | H | phenethyl | |
| Ib-84 | S | Cl | H | H | benzyl | |
| Ib-85 | S | Cl | H | H | 4-methoxy-benzyl | |
| Ib-86 | S | Cl | H | H | 4-chloro-benzyl | |
| Ib-87 | S | Cl | H | H | 3-methylsulfanyl-propyl | |
| Ib-88 | S | Cl | H | H | butyl | |
| Ib-89 | S | Cl | H | H | 3,5-dichloro-benzyl | |
| Ib-90 | S | Cl | H | H | pyridin-2-ylmethyl | |
| Ib-91 | S | Cl | H | H | pyridin-3-ylmethyl | |
| Ib-92 | S | Cl | H | H | pyridin-4-ylmethyl | |
| Ib-93 | S | Cl | H | H | tetrahydro-pyran-4-ylmethyl | |
| Ib-94 | S | Cl | H | H | 2-chloro-thiazol-5-ylmethyl | |
| Ib-95 | S | H | Cl | H | butyl | |
| Ib-96 | S | H | Cl | H | 2-methyl-propyl | |
| Ib-97 | S | H | Cl | H | cyclopropyl-methyl | |
| Ib-98 | S | H | Cl | H | tetrahydro-thiopyran-3-yl- | |
| | | | | | methyl | |
| Ib-99 | S | H | Cl | H | phenethyl | |
| Ib-100 | S | H | Cl | H | benzyl | |
| Ib-101 | S | H | Cl | H | 3,5-dichloro-benzyl | |
| Ib-102 | S | H | Cl | H | 4-methoxy-benzyl | |
| Ib-103 | S | H | Cl | H | 4-chloro-benzyl | |
| Ib-104 | S | H | Cl | H | 3-methylsulfanyl-propyl | |
| Ib-105 | S | H | Cl | H | pyridin-2-ylmethyl | |
| Ib-106 | S | H | Cl | H | pyridin-3-ylmethyl | |
| Ib-107 | S | H | Cl | H | pyridin-4-ylmethyl | |
| Ib-108 | S | H | Cl | H | tetrahydro-pyran-4-ylmethyl | |
| Ib-109 | S | H | Cl | H | 2-chloro-thiazol-5-ylmethyl | |
| Ib-110 | S | H | Cl | H | 2-chloro-benzyl | |
| Ib-111 | S | H | Cl | H | 3-chloro-benzyl | |
| Ib-112 | S | H | Cl | H | 2-methoxy-benzyl | |
| Ib-113 | S | H | Cl | H | 3-methoxy-benzyl | |
| Ib-114 | S | H | Cl | CH₃ | 4-fluoro-benzyl | |
| Ib-115 | S | H | Cl | CH₃ | 4-bromo-benzyl | |
| Ib-116 | S | H | Cl | CH₃ | 4-chloro-benzyl | |
| Ib-117 | S | H | Cl | H | 4-trifluoromethyl-benzyl | |
| Ib-118 | S | H | Cl | H | 4-cyano-benzyl | |
| Ib-119 | S | H | Cl | CH₃ | 2-methyl-propyl | |
| Ib-120 | S | H | Cl | CH₃ | 2,2-dimethylpropyl | |
| Ib-121 | S | H | Cl | CH₃ | 3-methyl-butyl | |
| Ib-122 | S | H | Cl | CH₃ | cyclopropyl-methyl | |
| Ib-123 | S | H | Cl | H | 4-dimethylamino-benzyl | |
| Ib-124 | S | H | Cl | H | 4-nitro-benzyl | |
| Ib-125 | S | H | Cl | H | 4-bromo-benzyl | |
| Ib-126 | S | H | Cl | H | 4-methoxycarbonyl-benzyl | |
| Ib-127 | S | H | Cl | H | 4-acetylamino-benzyl | |
| Ib-128 | S | H | Cl | H | 1-phenylethynyl-butyl | |
| Ib-129 | S | H | Cl | H | 4-fluoro-benzyl | |
| Ib-130 | S | H | Cl | H | 1-phenyl-prop-2-ynyl | |
| Ib-131 | S | H | Cl | H | 4,4-dimethyl-1-phenyl-pent-2- | |
| | | | | | ynyl | |
| Ib-132 | S | H | Cl | H | 1-(4-methoxy-phenyl)-hex-2- | |
| | | | | | ynyl | |
| Ib-133 | S | H | Cl | H | 1-phenyl-3-trimethylsilanyl- | |
| | | | | | prop-2-ynyl | |
| Ib-134 | S | H | Cl | H | 4-methoxy-1-phenyl-but-2- | |
| | | | | | ynyl | |
| Ib-135 | S | H | Cl | H | 4-ethoxycarbonyl-1-phenyl- | |
| | | | | | but-2-ynyl | |
| Ib-136 | S | H | Cl | H | 1-phenyl-hex-2-ynyl | |
| Ib-137 | S | H | Cl | H | 1-(4-chloro-phenyl)-hex-2- | |
| | | | | | ynyl | |
| Ib-138 | S | Cl | Cl | H | 2-methyl-propyl | |
| Ib-139 | R | H | Cl | H | 2-methyl-propyl | |
| Ib-140 | S | H | Cl | H | 1-phenyl-3-(pyrrolidin-2-yl)- | |
| | | | | | prop-2-ynyl | |
| Ib-141 | S | H | Cl | H | 1-(4-chlorophenyl)-prop-2- | |
| | | | | | ynyl | |
| Ib-142 | S | H | Cl | H | 1-(3-chlorophenyl)-prop-2- | |
| | | | | | ynyl | |
| Ib-143 | S | H | Cl | H | 1-(2-chlorophenyl)-prop-2- | |
| | | | | | ynyl | |
| Ib-144 | S | H | Cl | H | 1-(3,5-dichlorophenyl)-prop- | |
| | | | | | 2-ynyl | |
| Ib-145 | S | Cl | Cl | H | 2-cyanoethyl | |
| Ib-146 | S | H | Cl | H | 1-(3-methoxyphenyl)-prop-2- | |
| | | | | | ynyl | |
| Ib-147 | S | H | Cl | H | 1-(2-methoxyphenyl)-prop-2- | |
| | | | | | ynyl | |
| Ib-148 | S | H | Cl | H | 1-(3,4-dichlorophenyl)-prop- | |
| | | | | | 2-ynyl | |
| Ib-149 | S | H | Cl | H | 1-(4-methylphenyl)-prop-2- | |
| | | | | | ynyl | |
| Ib-150 | S | H | Cl | H | 1-(3-methylphenyl)-prop-2- | |
| | | | | | ynyl | |
| Ib-151 | S | H | Cl | H | 1-(2-methylphenyl)-prop-2- | |
| | | | | | ynyl | |
| Ib-152 | S | H | Cl | H | 1-(4-trifluoromethylphenyl)- | |
| | | | | | prop-2-ynyl | |
| Ib-153 | S | H | Cl | H | 1-(3-trifluoromethylphenyl)- | |
| | | | | | prop-2-ynyl | |
| Ib-154 | S | H | Cl | H | 1-(2-trifluoromethylphenyl)- | |
| | | | | | prop-2-ynyl | |
| Ib-155 | S | H | Cl | H | 1-(4-trifluoromethoxyphenyl)- | |
| | | | | | prop-2-ynyl | |
| Ib-156 | S | H | Cl | H | 1-(3-cyanophenyl)-prop-2- | |
| | | | | | ynyl | |
| Ib-157 | S | H | Cl | H | 1-(2-cyanophenyl)-prop-2- | |
| | | | | | ynyl | |
| Ib-158 | S | H | Cl | H | 1-(thiophen-3-yl)-prop-2-ynyl | |
| Ib-159 | S | H | Cl | H | 1-(tetrahydro-3-yl)-prop- | |
| | | | | | 2-ynyl | |
| Ib-160 | S | H | Cl | H | 1-pyridin-3-yl-prop-2-ynyl | |
| Ib-161 | S | H | Cl | H | 1-isopropyl-prop-2-ynyl | |
| Ib-162 | S | H | Cl | H | 1-ethynyl-3-methyl-prop-2- | |
| | | | | | ynyl | |
| Ib-163 | S | H | Cl | H | 1-(n-propyl)-prop-2-ynyl | |
| Ib-164 | S | H | Cl | CH₃ | 1-phenyl-3-trimethylsilanyl- | |
| | | | | | prop-2-ynyl | |
| Ib-165 | S | H | H | CH₃ | 1-phenyl-3-trimethylsilanyl- | |
| | | | | | prop-2-ynyl | |
| Ib-166 | S | H | Cl | CH₃ | 1-phenyl-prop-2-ynyl | |
| Ib-167 | S | H | H | CH₃ | 1-phenyl-prop-2-ynyl | |
| Ib-168 | S | H | H | H | 4-methoxy-1-phenyl-but-2- | |
| | | | | | ynyl | |
| Ib-169 | S | H | Cl | CH₃ | 4-methoxy-1-phenyl-but-2- | |
| | | | | | ynyl | |
| Ib-170 | S | H | H | CH₃ | 4-methoxy-1-phenyl-but-2- | |
| | | | | | ynyl | |
| Ib-171 | S | H | H | CH₃ | (5-methyl-1H-imidazol-4-yl)- | |
| | | | | | methyl | |
| Ib-172 | S | H | H | CH₃ | tetrahydropyran-4-yl-methyl | |
| Ib-173 | S | H | H | CH₃ | 3-dimethylaminomethyl- | |
| | | | | | tetrahydrofuran-3-ylmethyl | |
| Ib-174 | S | H | Cl | H | 2,3-dichloropropyl | |
| Ib-175 | S | Cl | H | H | 1-methyl-1H-pyrrol-2-yl- | |
| | | | | | methyl | |
| Ib-176 | S | H | Cl | H | 2,3-diacetoxy-propyl | |
| Ib-177 | S | H | Cl | H | 2-acetoxy-butyl | |
| Ib-178 | S | H | Cl | H | 1-methyl-2-phenyl-ethyl | |
| Ib-179 | S | H | Cl | H | 9H-fluoren-9-yl | |
| Ib-180 | S | H | Cl | H | cyclopentyl | |
| Ib-181 | R | H | Cl | H | tetrahydropyran-3-yl-methyl | |
| Ib-182 | R | H | Cl | H | 2,5-dihydro-thiophen-3- | |
| | | | | | ylmethyl | |
| Ib-183 | S | H | Cl | H | thiophen-2-ylmethyl | |
| Ib-184 | S | H | Cl | H | 3-isobutyl-isoxazol-5-ylmethyl | |
| Ib-185 | S | H | Cl | H | 1-(4-chloro-phenyl)-1H-pyrrol- | |
| | | | | | 3-ylmethyl | |

Ex = example

### Examples for the Action Against Harmful Pests

1. Activity Against Cotton Aphid (Aphis gossypii) The active compounds were formulated in 50:50 acetone:water and 100 ppm Kinetic™ surfactant.

Cotton plants at the cotyledon stage (one plant per pot) were infested by placing a heavily infested leaf from the main colony on top of each cotyledon. The aphids were allowed to transfer to the host plant overnight, and the leaf used to transfer the aphids was removed. The cotyledons were dipped in the test solution and allowed to dry. After 5 days, mortality counts were made.

In this test, compounds Ia-1, Ia-2, Ia-3, Ia-6, Ia-7, Ia-8, Ia-10, Ia-12, Ia-13, Ia-14, Ia-15, Ia-16, Ia-17, Ia-18, Ia-19, Ia-20, Ia-21, Ia-22, Ia-23, Ia-24, Ia-25, Ia-26, Ia-27, Ia-28, Ia-29, Ia-30, Ia-31, Ia-32, Ia-33, Ia-34, Ia-35, Ia-36, Ia-37, Ia-38, Ia-39, Ia-40, Ia-41, Ia-42, Ia-43, Ia-45, Ia-46, Ia-47, Ia-48, Ia-50, Ia-52, Ia-53, Ia-55, Ia-56, Ia-57, Ia-58, Ia-59, Ia-60, Ia-61, Ia-63, Ia-64, Ia-65, Ia-66, Ia-67, Ia-68, Ia-69, Ia-70, Ia-71, Ia-72, Ia-73, Ia-74, Ia-75, Ia-76, Ia-77, Ia-78, Ia-79, Ia-80, Ia-81, Ia-82, Ia-83, Ia-85, Ia-86, Ia-87, Ia-88, Ia-89, Ia-90, Ia-91, Ia-93, Ia-94, Ia-95, Ia-97, Ia-98, Ia-99, Ia-101, Ia-102, Ia-104, Ia-105, Ia-106, Ia-107, Ia-108, Ia-109, Ia-110, Ia-111, Ia-113, Ia-114, Ia-115, Ia-116, Ia-124, Ia-125, Ia-126, Ia-133, Ia-140, Ia-142, Ia-144, Ia-146, Ia-148, Ia-149, Ia-151, Ia-152, Ia-153, Ia-156, Ia-157, Ia-158, Ia-159, Ia-160, Ia-161, Ia-162, Ia-163, Ia-164, Ia-166, Ia-167, Ia-168, Ia-169, Ia-170, Ia-171, Ia-173, Ia-175, Ia-176, Ia-177, Ia-178, Ia-179, Ia-180, Ia-182, Ia-183, Ia-184, Ia-185, Ia-186, Ia-187, Ia-188, Ia-189, Ia-190, Ia-191, Ia-192, Ia-193, Ia-194, Ia-195, Ia-196, Ia-197, Ia-198, Ia-199, Ia-200, Ia-201, Ia-202, Ia-203, Ia-204, Ia-205, Ia-206, Ia-207, Ia-208, Ia-209, Ia-210, Ia-211, Ia-212, Ia-213, Ia-214, Ia-215, Ia-216, Ia-217, Ia-218, Ia-219, Ia-220, Ia-222, Ia-223 and Ia-232- of table I at 300 ppm showed over 90% mortality in comparison with untreated controls.

2. Green Peach Aphid (Myzus persicae)

The active compounds were formulated in 50:50 acetone:water and 100 ppm Kinetic™ surfactant.

Pepper plants in the 2^{nd} leaf-pair stage (variety ‘California Wonder’) were infested with approximately 40 laboratory-reared aphids by placing infested leaf sections on top of the test plants. The leaf sections were removed after 24 hr. The leaves of the intact plants were dipped into gradient solutions of the test compound and allowed to dry. Test plants were maintained under fluorescent light (24 hour photoperiod) at about 25° C. and 20-40% relative humidity. Aphid mortality on the treated plants, relative to mortality on check plants, was determined after 5 days.

In this test, compounds Ia-1, Ia-2, Ia-3, Ia-4, Ia-6, Ia-7, Ia-8, Ia-9, Ia-10, Ia-12, Ia-14, Ia-15, Ia-16, Ia-17, Ia-18, Ia-19, Ia-20, Ia-21, Ia-22, Ia-23, Ia-24, Ia-25, Ia-26, Ia-27, Ia-28, Ia-29, Ia-30, Ia-31, Ia-32, Ia-33, Ia-34, Ia-35, Ia-36, Ia-37, Ia-38, Ia-39, Ia-40, Ia-41, Ia-42, Ia-43, Ia-44, Ia-47, Ia-48, Ia-50, Ia-52, Ia-53, Ia-55, Ia-56, Ia-57, Ia-58, Ia-60, Ia-61, Ia-62, Ia-63, Ia-64, Ia-65, Ia-66, Ia-67, Ia-68, Ia-69, Ia-70, Ia-71, Ia-72, Ia-73, Ia-74, Ia-75, Ia-76, Ia-77, Ia-78, Ia-79, Ia-81, Ia-82, Ia-83, Ia-84, Ia-85, Ia-86, Ia-87, Ia-88, Ia-89, Ia-90, Ia-91, Ia-92, Ia-93, Ia-94, Ia-95, Ia-96, Ia-97, Ia-98, Ia-99, Ia-100, Ia-101, Ia-104, Ia-105, Ia-106, Ia-107, Ia-108, Ia-109, Ia-110, Ia-111, Ia-112, Ia-113, Ia-114, Ia-116, Ia-118, Ia-119, Ia-120, Ia-121, Ia-123, Ia-124, Ia-125, Ia-126, Ia-127, Ia-128, Ia-129, Ia-130, Ia-132, Ia-133, Ia-134, Ia-135, Ia-136, Ia-137, Ia-138, Ia-139, Ia-140, Ia-141, Ia-142, Ia-143, Ia-144, Ia-145, Ia-146, Ia-147, Ia-148, Ia-149, Ia-150, Ia-151, Ia-152, Ia-153, Ia-154, Ia-156, Ia-157, Ia-158, Ia-159, Ia-160, Ia-161, Ia-162, Ia-163, Ia-164, Ia-165, Ia-166, Ia-167, Ia-168, Ia-169, Ia-170, Ia-171, Ia-173, Ia-174, Ia-175, Ia-176, Ia-177, Ia-178, Ia-179, Ia-180, Ia-182, Ia-183, Ia-184, Ia-185, Ia-186, Ia-187, Ia-188, Ia-189, Ia-190, Ia-191, Ia-192, Ia-193, Ia-194, Ia-195, Ia-196, Ia-197, Ia-198, Ia-199, Ia-200, Ia-201, Ia-202, Ia-203, Ia-204, Ia-205, Ia-206, Ia-207, Ia-208, Ia-209, Ia-210, Ia-211, Ia-212, Ia-213, Ia-214, Ia-215, Ia-216, Ia-217, Ia-218, Ia-219, Ia-220, Ia-222, Ia-223, Ia-224, Ia-225, Ia-228, Ia-232 of table I, compounds Ib-13, Ib-17, Ib-22, Ib-36 and Ib-43 of table II at 300 ppm showed over 90% mortality in comparison with untreated controls

3. Activity Against Cowpea Aphid (Aphis craccivora)

The active compounds were formulated in 50:50 acetone:water. Potted cowpea plants colonized with 100-150 aphids of various stages were sprayed after the pest population has been recorded. Population reduction was recorded after 24, 72, and 120 hours.

In this test, compounds Ia-1, Ia-2, Ia-3, Ia-6, Ia-10, Ia-12, Ia-13, Ia-14, Ia-15, Ia-19, Ia-22, Ia-23, Ia-25, Ia-26, Ia-28, Ia-29, Ia-31, Ia-32, Ia-35, Ia-37, Ia-38, Ia-40, Ia-41, Ia-42, Ia-43, Ia-44, Ia-45, Ia-46, Ia-47, Ia-48, Ia-50, Ia-52, Ia-53, Ia-55, Ia-56, Ia-57, Ia-58, Ia-60, Ia-61, Ia-63, Ia-64, Ia-65, Ia-66, Ia-67, Ia-68, Ia-73, Ia-74, Ia-79, Ia-80, Ia-81, Ia-82, Ia-83, Ia-86, Ia-87, Ia-88, Ia-91, Ia-96, Ia-97, Ia-98, Ia-99, Ia-101, Ia-104, Ia-106, Ia-107, Ia-108, Ia-109, Ia-110, Ia-112, Ia-114, Ia-115, Ia-116, Ia-118, Ia-157, Ia-161, Ia-164, Ia-165, Ia-166, Ia-167, Ia-168, Ia-170, Ia-171, Ia-173, Ia-174, Ia-175, Ia-178, Ia-184, Ia-185, Ia-189, Ia-191, Ia-192, Ia-199, Ia-206, Ia-207, Ia-208, Ia-209, Ia-212, Ia-213, Ia-215, Ia-216, Ia-217, Ia-218, Ia-219, Ia-220, Ia-221, Ia-222, Ia-223, Ia-224, Ia-225, Ia-228, Ia-229, Ia-232 of table I at 300 ppm showed over 90% mortality in comparison with untreated controls.

4. Activity Against Silverleaf Whitefly (Bemisia argentifolii)

The active compounds were formulated in 50:50 acetone:water and 100 ppm Kinetic™ surfactant.

Selected cotton plants were grown to the cotyledon state (one plant per pot). The cotyledons were dipped into the test solution to provide complete coverage of the foliage and placed in a well-vented area to dry. Each pot with treated seedling was placed in a plastic cup and 10 to 12 whitefly adults (approximately 3-5 day old) were introduced. The insects were collected using an aspirator and an 0.6 cm, non-toxic Tygon™ tubing (R-3603) connected to a barrier pipette tip. The tip, containing the collected insects, was then gently inserted into the soil containing the treated plant, allowing insects to crawl out of the tip to reach the foliage for feeding. The cups were covered with a re-usable screened lid (150 micron mesh polyester screen PeCap from Tetko Inc). Test plants were maintained in the holding room at about 25° C. and 20-40% relative humidity for 3 days avoiding direct exposure to the fluorescent light (24 hour photoperiod) to prevent trapping of heat inside the cup. Mortality was assessed 3 days after treatment of the plants.

In this test, compounds Ia-5, Ia-6, Ia-7, Ia-8, Ia-15, Ia-16, Ia-17, Ia-18, Ia-19, Ia-21, Ia-22, Ia-23, Ia-26, Ia-31, Ia-33, Ia-35, Ia-37, Ia-38, Ia-39, Ia-40, Ia-41, Ia-42, Ia-43, Ia-45, Ia-47, Ia-48, Ia-50, Ia-52, Ia-57, Ia-58, Ia-59, Ia-60, Ia-61, Ia-64, Ia-68, Ia-79, Ia-80, Ia-81, Ia-82, Ia-83, Ia-85, Ia-86, Ia-87, Ia-88, Ia-91, Ia-93, Ia-94, Ia-95, Ia-96, Ia-97, Ia-99, Ia-104, Ia-105, Ia-106, Ia-127, Ia-136, Ia-138, Ia-142, Ia-147, Ia-148, Ia-157, Ia-159, Ia-162, Ia-175, Ia-176, Ia-177, Ia-178, Ia-179, Ia-182, Ia-183, Ia-184, Ia-199, Ia-212, Ia-213, Ia-214, Ia-215, Ia-217, Ia-219, Ia-224, Ia-229, Ia-230, Ia-232 of table I, compounds Ib-29, Ib-30, Ib-32, Ib-34, Ib-36 and Ib-40 of table II at 300 ppm showed over 75% mortality in comparison with untreated controls.

5. Orchid Thrips (Dichromothrips corbetti)

Dichromothrips corbetti adults used for bioassay were obtained from a colony maintained continuously under laboratory conditions. For testing purposes, the test compound was diluted to a concentration of 300 ppm (wt compound: vol diluent) in a 1:1 mixture of acetone:water, plus 0.01% Kinetic® surfactant.

Thrips potency of each compound was evaluated by using a floral-immersion technique. Plastic petri dishes were used as test arenas. All petals of individual, intact orchid flowers were dipped into treatment solution for approximately 3 seconds and allowed to dry for 2 hours. Treated flowers were placed into individual petri dishes along with 10-15 adult thrips. The petri dishes were then covered with lids. All test arenas were held under continuous light and a temperature of about 28° C. for duration of the assay. After 4 days, the numbers of live thrips were counted on each flower, and along inner walls of each petri dish. The level of thrips mortality was extrapolated from pre-treatment thrips numbers.

In this test, compounds of Ia-2, Ia-6, Ia-7, Ia-9, Ia-10, Ia-14, Ia-20, Ia-22, Ia-23, Ia-26, Ia-31, Ia-34, Ia-38, Ia-42, Ia-45, Ia-48, Ia-51, Ia-52, Ia-55, Ia-56, Ia-63, Ia-65, Ia-67, Ia-96, Ia-106, Ia-175, Ia-212, Ia-213, Ia-215, Ia-217, Ia-218, Ia-219, Ia-222, Ia-226, Ia-227, Ia-229, Ia-230, Ia-232 of table I at 300 ppm showed over 75% mortality in comparison with untreated controls.

6. Activity Against Colorado Potato Beetle (Leptinotarsa decemlineata)

Potato plants were utilized for bioassays. Excised plant leaves were dipped into 1:1 acetone/water dilutions of the active compounds. After the leaves had dried, they were individually placed onto water-moistened filter paper on the bottoms of Petri dishes. Each dish was infested with 5 to 7 larvae and covered with a lid. Each treatment dilution was replicated 4 times. Test dishes were held at approximately 27° C. and 60% humidity. Numbers of live and morbid larvae were assessed in each dish at 5 days after treatment application, and percent mortality was calculated.

In this test, the compounds Ia-10, Ia-11, Ia-14, Ia-22, Ia-23, Ia-44, Ia-50, Ia-52, Ia-59, Ia-65, Ia-67, Ia-73, Ia-76, Ia-88, Ia-121, Ia-126, Ia-128, Ia-141, Ia-142, Ia-143, Ia-153, Ia-155, Ia-159, Ia-161, Ia-188, Ia-201, Ia-232 of table I, compounds Ib-4, Ib-19, Ib-20 and Ib-40 of table II at 300 ppm showed over 50% mortality compared to 0% mortality of untreated controls.

1-30. (canceled)

31. A method of combating pest, which method comprises
contacting said pest or their food supply, habitat, breeding ground or their locus with a 3-pyridyl compound of formula I or a salt thereof, 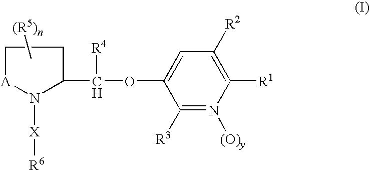
wherein
A is a chemical bond, CH₂ or a group CHR^{5a};
X is a chemical bond or C₁-C₃-alkylene, which may carry 1 or 2 identical or different radicals R⁷;
y is 0 or 1;
n is 0, 1 or 2;
R¹ and R² are each independently selected from the group consisting of hydrogen, halogen, cyano, nitro, C₁-C₆-alkyl, C₁-C₆-alkyl, which may be substituted by one OH, phenyl or CN radical, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, C₁-C₆-alkyl-C₃-C₈-cycloalkyl, ORⁱ, SRⁱ, S(═O)Rⁱ, S(═O)₂Rⁱ, NRⁱR^{j}, NR^{j}C(═O)Rⁱ, C(═O)Rⁱ, O—C(O)Rⁱ, C(═O)ORⁱ, SiRⁱ_{z}R^{j}_{3-z} with z being 0, 1, 2 or 3, phenyl and a 3-, 4-, 5-, 6- or 7-membered heterocycle containing 1, 2, 3 or 4 heteroatoms selected from the group consisting of oxygen, nitrogen and sulfur, wherein phenyl and said heterocycle may be unsubstituted or may carry 1, 2, or 3 substituents selected from the group consisting of halogen, amino, cyano, Rⁱ, ORⁱ, SRⁱ, NRⁱR^{j}, NR^{j}C(═O)Rⁱ, C(═O)Rⁱ, O—C(O)Rⁱ, C(═O)ORⁱ, SO₂Rⁱ, and nitro;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, C₁-C₆-haloalkoxy, C₁-C₄-alkoxy-C₁-C₆-alkyl, C₁-C₄-alkylthio-C₁-C₆-alkyl, C₁-C₄-alkylsulfinyl-C₁-C₆-alkyl, C₁-C₄-alkylsulfonyl-C₁-C₆-alkyl, C₁-C₄-haloalkoxy-C₁-C₆-alkyl, C₁-C₄-haloalkylthio-C₁-C₆-alkyl, (C₁-C₄-alkoxy)carbonyl-C₁-C₆-alkyl, di(C₁-C₄-alkyl)amino-C₁-C₆-alkyl, C₃-C₆-cycloalkyl-C₁-C₆-alkyl, phenyl-C₁-C₆-alkyl, or C₁-C₆-alkyl which is substituted with 1 to 3 cyano groups;
R² may also be a radical of the formula Z—R^{y}, wherein Z is selected from the group consisting of —C≡C—(C₀-C₆-alkylene)-, —(CH═CH)ₚ—(C₀-C₆-alkylene)- with p being 1 or 2, —CH═CH—C(O)—(C₀-C₆-alkylene)-, —C(O)—(C₀-C₆-alkylene)- and —CH₂—Z′—C(O)—(C₀-C₆-alkylene)- with Z′ being CH₂ or NH and R^{y} is selected from the group consisting of hydrogen, C₁-C₈-alkyl, C₁-C₆-haloalkyl, C₁-C₆-hydroxyalkyl, C₁-C₆-alkoxy, amino, C₁-C₆-alkylamino, C₁-C₆-dialkylamino, 5 to 7 membered, azaheterocyclyl, attached to Z via its nitrogen atom, phenyl, naphthyl, biphenyl, furyl, thienyl, pyridinyl, pyridazinyl, pyrimidinyl, pyrrolyl, pyrazolyl, imidazolyl, indolyl, thiazolyl, oxazolyl, isoxazolyl, thiadiazolyl, oxadiazolyl, quinolinyl, and isoquinolyl, wherein alkyl and the 19 last mentioned radicals may carry one or two substituents selected from the group consisting of C₁-C₆-alkyl, C₁-C₆-haloalkyl, C₁-C₆-alkoxy-C₁-C₆-alkyl, C₁-C₆-alkoxy, C₁-C₆-alkoxy-C₁-C₆-alkoxy, halogen, cyano, hydroxy, amino, C₁-C₆-alkylamino, carboxyl and C₁-C₆-alkoxycarbonyl;
R³ is hydrogen, halogen or C₁-C₆-alkyl;
R⁴ is hydrogen or C₁-C₆-alkyl;
R⁵ is selected from the group consisting of halogen, OH, cyano, C₁-C₆-alkyl and C₁-C₆-haloalkyl and for n being 2 R⁵ may be identical or different;
R^{5a} is selected from the group consisting of halogen, cyano, C₁-C₆-alkyl and C₁-C₆-haloalkyl;
R⁶ is C₁-C₁₀-alkyl, C₂-C₁₀-alkenyl, C₂-C₁₀-alkynyl, wherein the last three mentioned radicals may be unsubstituted or may carry 1, 2 or 3 identical or different radicals R^{a},
C₁-C₁₀-haloalkyl, C₂-C₁₀-haloalkenyl, C₂-C₁₀-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from the group consisting of oxygen, nitrogen and sulfur, wherein C₃-C₈-cycloalkyl, C₃-C₈-cycloalkenyl, phenyl and said heterocycle may be unsubstituted or may carry 1, 2, 3, 4 or 5 substituents R^{b},
wherein C₃-C₈-cycloalkyl, C₃-C₈-cycloalkenyl, phenyl and said heterocycle may also be fused to 1 or 2 rings selected from the group consisting of phenyl and a 5- to 6-membered heterocycle which may contain 1, 2 or 3 heteroatoms selected from oxygen, nitrogen and sulfur,
R^{a} is selected from the group consisting of cyano, nitro, C₃-C₈-cycloalkyl, C₃-C₈-halocycloalkyl, C₃-C₈-cycloalkenyl, C₃-C₈-halocycloalkenyl, OR^{k}, SR^{k}, S(═O)R^{k}, S(═O)₂R^{k}, N(OR^{k})R^{m}, S(═O)₂NR^{m}Rⁿ, O—C(═O)R^{k}, C(═O)R^{k}, C(═O)OR^{k}, C(═O)NR^{m}Rⁿ, C(═NOR^{k})R^{m}, NR^{k}C(=G)R^{m}, N(OR^{k})C(=G)R^{m}, N[C(=G)R¹][C(=G)R^{m}], NR^{k}C(=G)OR^{m}, N(OR^{k})C(=G)OR^{m}, C(=G)NR^{k}—NR^{m}Rⁿ, NR^{k}SO₂R^{m}, PR^{m}Rⁿ, SiR^{k}_{z}R^{m}_{3-z} with z being 0, 1, 2 or 3, phenyl and a 3-, 4-, 5-, 6- or 7-membered heterocycle containing 1, 2, 3 or 4 heteroatoms selected from the group consisting of oxygen, nitrogen and sulfur, wherein said phenyl or said heterocycle may be unsubstituted or may carry 1, 2, 3, 4 or 5 radicals selected from halogen and C₁-C₄-alkyl;
G is oxygen or sulfur;
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, phenyl, benzyl or a 3-, 4-, 5-, 6- or 7-membered heterocycle containing 1, 2, 3 or 4 heteroatoms as ring members, selected from the group consisting of oxygen, nitrogen and sulfur, wherein C₃-C₈-cycloalkyl, C₃-C₈-cycloalkenyl, phenyl, benzyl and said heterocycle may be unsubstituted or may carry 1, 2, 3, 4 or 5 radicals selected from halogen and C₁-C₄-alkyl,
or R^{m} and Rⁿ together with the nitrogen, to which they are bound may form an N-bound 5-, 6- or 7-membered heterocycle, which in addition to the nitrogen atom may contain a further heteroatom selected from the group consisting of oxygen, nitrogen and sulphur,
R^{b} is halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, benzyl or a radical R^{a};
or, if X is not a chemical bond, R⁶ may also be a radical of the formulae OR^{c}, SR^{c}, S(═O)R^{c}, S(═O)₂R^{c}, NR^{d}R^{e}, N(OR^{c})R^{d}, S(═O)₂NR^{d}R^{e}, C(═O)R^{c}, C(═O)OR^{c}, O—C(═O)R^{c}, C(═O)NR^{d}R^{e}, C(═O)NR^{c}—NR^{d}R^{e}, C(═NOR^{c})R^{d}, C(═S)R^{c}, C(═S)OR^{c}, C(═S)NR^{d}R^{e}, NR^{d}C(=G)R^{c}, N(OR^{c})C(=G)R^{d}, N[C(=G)R^{c}][C(=G)R^{d}], NR^{d}C(=G)OR^{c}, N(OR^{c})C(=G)OR^{d}, NR^{k}SO₂R^{m} or SiR^{k}_{z}R^{m}_{3-z} with z being 0, 1, 2 or 3,
R^{c}, R^{d}, R^{e} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, phenyl, benzyl or a 3-, 4-, 5-, 6- or 7-membered heterocycle containing 1, 2, 3 or 4 heteroatoms a ring members, selected from the group consisting of oxygen, nitrogen and sulfur, wherein C₃-C₆-cycloalkyl, C₃-C₆-cycloalkenyl, phenyl, benzyl and said heterocycle may be unsubstituted or may carry 1, 2, 3, 4 or 5 radicals selected from halogen and C₁-C₄-alkyl,
or R^{d} and R^{e} together with the nitrogen, to which they are bound may form an N-bound 5-, 6- or 7-membered heterocycle, which in addition to the nitrogen atom may contain a further heteroatom selected from the group consisting of oxygen, nitrogen and sulphur;
R⁷ is hydrogen, C₁-C₆-alkyl, C₂-C₆-alkenyl, C₂-C₆-alkynyl, wherein the last three mentioned radicals may be unsubstituted or may carry 1, 2 or 3 identical or different radicals R^{f}, wherein R^{f} has the meanings given for R^{a},
C₁-C₆-haloalkyl, C₂-C₆-haloalkenyl, C₂-C₆-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from the group consisting of oxygen, nitrogen and sulphur, it being possible for said heterocycle to contain one carbonyl group as ring member, wherein C₃-C₆-cycloalkyl, C₃-C₆-cycloalkenyl, phenyl and said heterocycle may be unsubstituted or may carry 1, 2, 3, 4 or 5 substituents R^{g}, wherein R^{g} has the meanings given for R^{b},
wherein C₃-C₈-cycloalkyl, C₃-C₈-cycloalkenyl, phenyl and said heterocycle may also be fused to a ring selected from the group consisting of phenyl and a 5- to 6-membered saturated, partially unsaturated or aromatic heterocycle which may contain 1, 2 or 3 heteroatoms selected from the group consisting of oxygen, nitrogen, sulfur,
and a radical of the formulae OR^{c}, SR^{c}, S(═O)R^{c}, S(═O)₂R^{c}, NR^{d}R^{e}, N(OR^{c})R^{d}, S(═O)₂NR^{d}R^{e}, C(═O)R^{c}, C(═O)OR^{c}, C(═O)NR^{d}R^{e}, C(═O)NR^{c}—NR^{d}R^{e}, C(═NOR^{c})R^{d}, C(═S)R^{c}, C(═S)OR^{c}, C(═S)NR^{d}R^{e}, NR^{d}C(=G)R^{c}, N(OR^{c})C(=G)R^{d}, N[C(=G)R^{c}][C(=G)R^{d}], NR^{d}C(=G)OR^{c}, N(OR^{c})C(=G)OR^{d}, NR^{k}SO₂R^{m}, SiR^{k}_{z}R^{m}_{3-z}, wherein G, n, R^{c}, R^{d}, R^{e}, R^{k} and R^{m} are as defined above.

32. The method of claim 31, wherein
R¹ and R² are each independently hydrogen, halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, C₁-C₆-alkyl-C₃-C₈-cycloalkyl, ORⁱ, SRⁱ, S(═O)Rⁱ, S(═O)₂Rⁱ, NRⁱR^{j}, NR^{j}C(═O)Rⁱ, C(═O)Rⁱ, O—C(O)Rⁱ, C(═O)ORⁱ, SiRⁱ_{z}R^{j}_{3-z} with z being 0, 1, 2 or 3,
phenyl or a 3-, 4-, 5-, 6- or 7-membered heterocycle containing 1, 2, 3 or 4 heteroatoms selected from the group consisting of oxygen, nitrogen and sulfur, wherein phenyl and said heterocycle may be unsubstituted or may carry 1, 2, or 3 substituents selected from the group consisting of halogen, amino, cyano, Rⁱ, ORⁱ, SRⁱ, NRⁱR^{j}, NR^{j}C(═O)Rⁱ, C(═O)Rⁱ, O—C(O)Rⁱ, C(═O)ORⁱ, SO₂Rⁱ, and nitro;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, C₁-C₆-haloalkoxy, C₁-C₄-alkoxy-C₁-C₆-alkyl, C₁-C₄-alkylthio-C₁-C₆-alkyl, C₁-C₄-alkylsulfinyl-C₁-C₆-alkyl, C₁-C₄-alkylsulfonyl-C₁-C₆-alkyl, C₁-C₄-haloalkoxy-C₁-C₆-alkyl, C₁-C₄-haloalkylthio-C₁-C₆-alkyl, (C₁-C₄-alkoxy)carbonyl-C₁-C₆-alkyl, di(C₁-C₄-alkyl)amino-C₁-C₆-alkyl, C₃-C₆-cycloalkyl-C₁-C₆-alkyl, phenyl-C₁-C₆-alkyl, or C₁-C₆-alkyl which is substituted with 1 to 3 cyano groups; and
R⁶ is C₁-C₁₀-alkyl, C₂-C₁₀-alkenyl, C₂-C₁₀-alkynyl, wherein the last three mentioned radicals may be unsubstituted or may carry 1, 2 or 3 identical or different radicals R^{a},
C₁-C₁₀-haloalkyl, C₂-C₁₀-haloalkenyl, C₂-C₁₀-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from the group consisting of oxygen, nitrogen and sulfur, wherein C₃-C₈-cycloalkyl, C₃-C₈-cycloalkenyl, phenyl and said heterocycle may be unsubstituted or may carry 1, 2, 3, 4 or 5 substituents R^{b},
wherein C₃-C₈-cycloalkyl, C₃-C₈-cycloalkenyl, phenyl and said heterocycle may also be fused to 1 or 2 rings selected from phenyl and a 5- to 6-membered heterocycle which may contain 1, 2 or 3 heteroatoms selected from the group consisting of oxygen, nitrogen and sulfur,
R^{a} is selected from the group consisting of cyano, nitro, C₃-C₈-cycloalkyl, C₃-C₈-halocycloalkyl, C₃-C₈-cycloalkenyl, C₃-C₈-halocycloalkenyl, OR^{k}, SR^{k}, S(═O)R^{k}, S(═O)₂R^{k}, NR^{m}Rⁿ, N(OR^{k})R^{m}, S(═O)₂NR^{m}Rⁿ, O—C(═O)R^{k}, C(═O)R^{k}, C(═O)OR^{k}, C(═O)NR^{m}Rⁿ, C(═NOR^{k})R^{m}, NR^{k}C(=G)R^{m}, N(OR^{k})C(=G)R^{m}, N[C(=G)R^{k}][C(=G)R^{m}], NR^{k}C(=G)OR^{m}, N(OR^{k})C(=G)OR^{m}, C(=G)NR^{k}—NR^{m}Rⁿ, NR^{k}SO₂R^{m}, PR^{m}Rⁿ, SiR^{k}_{z}R^{m}_{3-z} with z being 0, 1, 2 or 3,
phenyl or a 3-, 4-, 5-, 6- or 7-membered heterocycle containing 1, 2, 3 or 4 heteroatoms selected from the group consisting of oxygen, nitrogen and sulfur, wherein said phenyl or said heterocycle may be unsubstituted or may carry 1, 2, 3, 4 or 5 radicals selected from halogen and C₁-C₄-alkyl;
G is oxygen or sulfur;
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, phenyl, benzyl or a 3-, 4-, 5-, 6- or 7-membered heterocycle containing 1, 2, 3 or 4 heteroatoms as ring members, selected from the group consisting of oxygen, nitrogen and sulfur, wherein C₃-C₈-cycloalkyl, C₃-C₈-cycloalkenyl, phenyl, benzyl and said heterocycle may be unsubstituted or may carry 1, 2, 3, 4 or 5 radicals selected from halogen and C₁-C₄-alkyl,
or R^{m} and Rⁿ together with the nitrogen, to which they are bound may form an N-bound 5-, 6- or 7-membered heterocycle, which in addition to the nitrogen atom may contain a further heteroatom selected from the group consisting of oxygen, nitrogen and sulphur,
R^{b} is halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl or a radical R^{a};
or, if X is not a chemical bond, R⁶ may also be a radical of the formulae OR^{c}, SR^{c}, S(═O)R^{c}, S(═O)₂R^{c}, NR^{d}R^{e}, N(OR^{c})R^{d}, S(═O)₂NR^{d}R^{e}, C(═O)R^{c}, C(═O)OR^{c}, O—C(═O)R^{c}, C(═O)NR^{d}R^{e}, C(═O)NR^{c}—NR^{d}R^{e}, C(═NOR^{c})R^{d}, C(═S)R^{c}, C(═S)OR^{c}, C(═S)NR^{d}R^{e}, NR^{d}C(=G)R^{c}, N(OR^{c})C(=G)R^{d}, N[C(=G)R^{c}][C(=G)R^{d}], NR^{d}C(=G)OR^{c}, N(OR^{c})C(=G)OR^{d}, NR^{k}SO₂R^{m} or SiR^{k}_{z}R^{m}_{3-z} with z being 0, 1, 2 or 3,
R^{c}, R^{d}, R^{e} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, phenyl, benzyl or a 3-, 4-, 5-, 6- or 7-membered heterocycle containing 1, 2, 3 or 4 heteroatoms a ring members, selected from the group consisting of oxygen, nitrogen and sulfur, wherein C₃-C₆-cycloalkyl, C₃-C₆-cycloalkenyl, phenyl, benzyl and said heterocycle may be unsubstituted or may carry 1, 2, 3, 4 or 5 radicals selected from the group consisting of halogen and C₁-C₄-alkyl,
or R^{d} and R^{e} together with the nitrogen, to which they are bound may form an N-bound 5-, 6- or 7-membered heterocycle, which in addition to the nitrogen atom may contain a further heteroatom selected from the group consisting of oxygen, nitrogen and sulphur.

33. The method of claim 31, wherein X in formula I is a radical CHR⁷ or CHR⁷—CH₂.

34. The method of claim 31, wherein X in formula I is a chemical bond.

35. The method of claim 31, wherein R⁶ in formula I is C₃-C₈-cycloalkyl, C₃-C₈-cycloalkenyl, phenyl or a 3-, 4-, 5-, 6- or 7-membered heterocycle containing 1, 2, 3 or 4 heteroatoms selected from the group consisting of oxygen, nitrogen and sulfur, wherein C₃-C₈-cycloalkyl, C₃-C₈-cycloalkenyl, phenyl and said heterocycle may be unsubstituted or may carry 1, 2, 3, 4 or 5 substituents R^{b}, it being also possible for C₃-C₈-cycloalkyl, C₃-C₈-cycloalkenyl, phenyl and said heterocycle to be fused to 1 or 2 rings selected from the group consisting of phenyl and a 5- to 6-membered saturated, partially unsaturated or aromatic heterocycle which may contain 1, 2 or 3 heteroatoms selected from the group consisting of oxygen, nitrogen and sulphur.

36. The method of claim 33, wherein
R⁶ in formula I is C₃-C₈-cycloalkyl, C₃-C₈-cycloalkenyl, phenyl or a 3-, 4-, 5-, 6- or 7-membered heterocycle containing 1, 2, 3 or 4 heteroatoms selected from the group consisting of oxygen, nitrogen and sulfur, wherein C₃-C₆-cycloalkyl, C₃-C₆-cycloalkenyl, phenyl and said heterocycle may be unsubstituted or may carry 1, 2, 3, 4 or 5 substituents R^{b}, it being also possible for C₃-C₈-cycloalkyl, C₃-C₈-cycloalkenyl, phenyl and said heterocycle to be fused to 1 or 2 rings selected from the group consisting of phenyl and a 5- to 6-membered saturated, partially unsaturated or aromatic heterocycle which may contain 1, 2 or 3 heteroatoms selected from the group consisting of oxygen, nitrogen and sulphur and
R⁷ is hydrogen, C₁-C₆-alkyl, C₂-C₆-alkenyl or C₂-C₆-alkynyl, wherein the last three mentioned radicals may be unsubstituted or may carry 1, 2 or 3 identical or different radicals R^{f} as defined above.

37. The method of claim 33, wherein R⁶ in formula I is phenyl which is unsubstituted or which carries 1, 2 or 3 substituents R^{b} as defined above.

38. The method of claim 33, wherein X is CH₂ and wherein R⁶ is selected from the group consisting of:
C₁-C₈-alkyl, which may carry 1 or 2 radicals selected from the group consisting of halogen, CN, OH, OR^{k}, SR^{k}, NH₂, NHC(O)R^{k} and OC(O)R^{k} with R^{k} being hydrogen, C₁-C₄-alkyl, C₁-C₄-haloalkyl, phenyl or pyridyl it being possibly for phenyl and pyridyl to carry 1 or 2 substituents, selected from the group consisting of halogen and C₁-C₄-alkyl;
C₂-C₈-alkenyl,
C₂-C₈-alkynyl,
C₃-C₈-cycloalkyl,
a saturated or partially unsaturated 5- or 6-membered heterocyclic radical selected from the group consisting of tetrahydrofuryl, dihydrofuryl, dihydropyranyl, tetrahydropyranyl, thianyl, dehydrothianyl, thiolanyl, and morpholinyl, 5- or 6-membered heteroaromatic radical selected from the group consisting of pyrrolyl, furyl, thienyl, imidazolyl, pyrazolyl, isoxazolyl, isothiazolyl, oxazolyl, thiazolyl and pyridyl, it being possible for the 5- or 6-membered heteroaromatic radical to carry 1 or 2 radicals, selected from the group consisting of halogen, C₁-C₆-alkyl, and phenyl, which may be unsubstituted or may carry 1, 2 or 3 halogen atoms;
phenyl, which may carry 1, 2 or 3 radicals selected from the group consisting of halogen, CN, OH, C₁-C₄-alkyl, C₁-C₄-haloalkyl, C₁-C₄-alkoxy, C₁-C₄-haloalkoxy, C₂-C₄-alkenyl, benzyl, phenoxy and benzyloxy, wherein said phenyl ring in the four last mentioned radicals may be unsubstituted or may carry 1, 2 or 3 halogen atoms, SR^{k}, S(O)₂R^{k}, NR^{k}R^{m}, C(O)R^{k}, CO(O)R^{k}, NHC(O)R^{k} and OC(O)R^{k} with R^{k} being hydrogen, C₁-C₄-alkyl, C₁-C₄-haloalkyl and phenyl, which may be unsubstituted or may carry 1, 2 or 3 halogen atoms.

39. The method of claim 34, wherein X—R⁶ in formula I is
C₁-C₁₀-alkyl, C₂-C₁₀-alkenyl, C₂-C₁₀-alkynyl, wherein the last three mentioned radicals may be unsubstituted or may carry 1, 2 or 3 identical or different radicals R^{a}, selected from cyano, nitro, C₃-C₈-cycloalkenyl, C₃-C₈-halocycloalkenyl, OR^{k}, SR^{k}, S(═O)R^{k}, S(═O)₂R^{k}, NR^{m}Rⁿ, N(OR^{k})R^{m}, S(═O)₂NR^{m}r, O—C(═O)R^{k}, C(═O)R^{k}, C(═O)OR^{k}, C(═O)NR^{m}Rⁿ, C(═NOR^{k})R^{m}, NR^{k}C(=G)R^{m}, N(OR^{k})C(=G)R^{m}, N[C(=G)R^{k}][C(=G)R^{m}], NR^{k}C(=G)OR^{m}, N(OR^{k})C(=G)OR^{m}, C(=G)NR^{k}—NR^{m}Rⁿ, NR^{k}SO₂R^{m}, PR^{m}Rⁿ and SiR^{k}_{z}R^{m}_{3-z} with z being 0, 1, 2 or 3, wherein R^{k}, R^{m} and Rⁿ are as defined above,
C₁-C₁₀-haloalkyl, C₂-C₁₀-haloalkenyl, C₂-C₁₀-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from the group consisting of oxygen, nitrogen and sulfur, wherein C₃-C₈-cycloalkyl, C₃-C₈-cycloalkenyl, phenyl and said heterocycle may be unsubstituted or may carry 1, 2, 3, 4 or 5 substituents R^{b}, as defined above.

40. The method of claim 31, wherein said radical X—R⁶ is of the formula 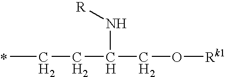
wherein * denotes the bonding position to the nitrogen of the heterocycle, R is hydrogen or a radical C(O)R^{k} and R^{k1} has one of the meanings given for R^{k} and wherein R^{k} is as defined above.

41. The method of claim 31, wherein X is CHR⁶R⁷ and wherein R⁶ is selected from
C₁-C₈-alkyl, which may carry 1 or 2 radicals selected from the group consisting of halogen, OH, OR^{k}, SR^{k}, NH₂ and NHC(O)R^{k} and OC(O)R^{k} with R^{k} being hydrogen, C₁-C₄-alkyl, C₁-C₄-haloalkyl, phenyl or pyridyl it being possible for phenyl and pyridyl to carry 1 or 2 substituents, selected from halogen and C₁-C₄-alkyl;
C₃-C₆-cycloalkyl,
a saturated or partially unsaturated 5- or 6-membered heterocyclic radical selected from the group consisting of tetrahydrofuryl, dihydrofuryl, dihydropyranyl, tetrahydropyranyl, thianyl, dehdrothianyl, thiolanyl, and morpholinyl,
5- or 6-membered heteroaromatic radical selected from the group consisting of pyrrolyl, furyl, thienyl, imidazolyl, pyrazolyl, isoxazolyl, isothiazolyl, oxazolyl, thiazolyl and pyridyl, it being possible for the 5- or 6-membered heteroaromatic radical to carry 1 or 2 radicals, selected from the group consisting of halogen, C₁-C₆-alkyl, phenyl, benzyl, phenoxy and benzyloxy, wherein said phenyl ring in the four last mentioned radicals may be unsubstituted or may carry 1, 2 or 3 halogen atoms;
phenyl, which may carry 1, 2 or 3 radicals selected from the group consisting of halogen, CN, OH, C₁-C₄-alkyl, C₁-C₄-haloalkyl, C₁-C₄-alkoxy, C₁-C₄-haloalkoxy, C₂-C₄-alkenyl, benzyl, phenoxy and benzyloxy, wherein said phenyl ring in the four last mentioned radicals may be unsubstituted or may carry 1, 2 or 3 halogen atoms, SR^{k}, S(O)₂R^{k}, NR^{k}R^{m}, C(O)R^{k}, CO(O)R^{k}, NHC(O)R^{k} and OC(O)R^{k} with R^{k} being hydrogen, C₁-C₄-alkyl, C₁-C₄-haloalkyl and phenyl, which may be unsubstituted or may carry 1, 2 or 3 halogen atoms;
and wherein R⁷ is C≡C—R^{f}, wherein R^{f} is as defined above.

42. The method of claim 31, wherein n in formula I is 0.

43. The method of claim 31, wherein A in formula I is CH₂.

44. The method of claim 31, wherein A in formula I is chemical bond.

45. The method of claim 31, wherein R¹ is selected from the group consisting of hydrogen, halogen, CN, NH₂, C₁-C₄-alkyl, which may be substituted by one OH or CN radical, C₁-C₄-haloalkyl, NHCHO and NHC(O)—C₁-C₄-alkyl.

46. The method of claim 31, wherein R² is selected from the group consisting of hydrogen, halogen, CN, NH₂, C₁-C₆-alkyl, which may be substituted by one OH, phenyl or CN radical, C₁-C₆-haloalkyl, C₂-C₆-alkenyl, C₂-C₆-alkynyl and NHC(O)—C₁-C₆-alkyl.

47. The method of claim 31, wherein R¹ and R² are each independently selected from the group consisting of halogen and hydrogen.

48. The method of claim 31, wherein R³ is hydrogen.

49. The method of claim 31, wherein y is 0.

50. A method for protecting growing plants from attack or infestation by pests comprising applying to the plant, or to the soil or artificial substrate or the water in which the plant is growing, a compound of formula I or salt thereof, 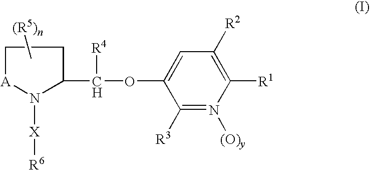
wherein
A is a chemical bond, CH₂ or a group CHR^{5a};
X is a chemical bond or C₁-C₃-alkylene, which may carry 1 or 2 identical or different radicals R⁷;
y is 0 or 1;
n is 0, 1 or 2;
R¹ and R² are each independently selected from the group consisting of hydrogen, halogen, cyano, nitro, C₁-C₆-alkyl, C₁-C₆-alkyl, which may be substituted by one OH, phenyl or CN radical, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, C₁-C₆-alkyl-C₃-C₈-cycloalkyl, ORⁱ, SRⁱ, S(═O)Rⁱ, S(═O)₂Rⁱ, NRⁱR^{j}, NR^{j}C(═O)Rⁱ, C(═O)Rⁱ, O—C(O)Rⁱ, C(═O)ORⁱ, SiRⁱ_{z}R^{j}_{3-z} with z being 0, 1, 2 or 3,
phenyl and a 3-, 4-, 5-, 6- or 7-membered heterocycle containing 1, 2, 3 or 4 heteroatoms selected from the group consisting of oxygen, nitrogen and sulfur, wherein phenyl and said heterocycle may be unsubstituted or may carry 1, 2, or 3 substituents selected from the group consisting of halogen, amino, cyano, Rⁱ, ORⁱ, SRⁱ, NRⁱR^{j}, NR^{j}C(═O)Rⁱ, C(═O)Rⁱ, O—C(O)Rⁱ, C(═O)ORⁱ, SO₂Rⁱ, and nitro;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, C₁-C₆-haloalkoxy, C₁-C₄-alkoxy-C₁-C₆-alkyl, C₁-C₄-alkylthio-C₁-C₆-alkyl, C₁-C₄-alkylsulfinyl-C₁-C₆-alkyl, C₁-C₄-alkylsulfonyl-C₁-C₆-alkyl, C₁-C₄-haloalkoxy-C₁-C₆-alkyl, C₁-C₄-haloalkylthio-C₁-C₆-alkyl, (C₁-C₄-alkoxy)carbonyl-C₁-C₆-alkyl, (C₁-C₄-alkyl)amino-C₁-C₆-alkyl, di(C₁-C₄-alkyl)amino-C₁-C₆-alkyl, phenyl-C₁-C₆-alkyl, or C₁-C₆-alkyl which is substituted with 1 to 3 cyano groups;
R² may also be a radical of the formula Z—R^{y}, wherein Z is selected from the group consisting of —C≡C—(C₀-C₆-alkylene)-, —(CH═CH)ₚ—(C₀-C₆-alkylene)- with p being 1 or 2, —CH═CH—C(O)—(C₀-C₆-alkylene)-, —C(O)—(C₀-C₆-alkylene)- and —CH₂—Z′—C(O)—(C₀-C₆-alkylene)- with Z' being CH₂ or NH and R^{y} is selected from the group consisting of hydrogen, C₁-C₈-alkyl, C₁-C₆-haloalkyl, C₁-C₆-hydroxyalkyl, C₁-C₆-alkoxy, amino, C₁-C₆-alkylamino, C₁-C₆-dialkylamino, 5 to 7 membered, azaheterocyclyl, attached to Z via its nitrogen atom, phenyl, naphthyl, biphenyl, furyl, thienyl, pyridinyl, pyridazinyl, pyrimidinyl, pyrrolyl, pyrazolyl, imidazolyl, indolyl, thiazolyl, oxazolyl, isoxazolyl, thiadiazolyl, oxadiazolyl, quinolinyl, and isoquinolyl, wherein alkyl and the 19 last mentioned radicals may carry one or two substituents selected from the group consisting of C₁-C₆-alkyl, C₁-C₆-haloalkyl, C₁-C₆-alkoxy-C₁-C₆-alkyl, C₁-C₆-alkoxy, C₁-C₆-alkoxy-C₁-C₆-alkoxy, halogen, cyano, hydroxy, amino, C₁-C₆-alkylamino, carboxyl and C₁-C₆-alkoxycarbonyl;
R³ is hydrogen, halogen or C₁-C₆-alkyl;
R⁴ is hydrogen or C₁-C₆-alkyl;
R⁵ is selected from the group consisting of halogen, OH, cyano, C₁-C₆-alkyl and C₁-C₆-haloalkyl and for n being 2 R⁵ may be identical or different;
R^{5a} is selected from the group consisting of halogen, cyano, C₁-C₆-alkyl and C₁-C₆-haloalkyl;
R⁶ is C₁-C₁₀-alkyl, C₂-C₁₀-alkenyl, C₂-C₁₀-alkynyl, wherein the last three mentioned radicals may be unsubstituted or may carry 1, 2 or 3 identical or different radicals R^{a},
C₁-C₁₀-haloalkyl, C₂-C₁₀-haloalkenyl, C₂-C₁₀-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from the group consisting of oxygen, nitrogen and sulfur, wherein C₃-C₈-cycloalkyl, C₃-C₈-cycloalkenyl, phenyl and said heterocycle may be unsubstituted or may carry 1, 2, 3, 4 or 5 substituents
wherein C₃-C₈-cycloalkyl, C₃-C₈-cycloalkenyl, phenyl and said heterocycle may also be fused to 1 or 2 rings selected from the group consisting of phenyl and a 5- to 6-membered heterocycle which may contain 1, 2 or 3 heteroatoms selected from oxygen, nitrogen and sulfur,
R^{a} is selected from the group consisting of cyano, nitro, C₃-C₈-cycloalkyl, C₃-C₈-halocycloalkyl, C₃-C₈-cycloalkenyl, C₃-C₈-halocycloalkenyl, OR^{k}, SR^{k}, S(═O)R^{k}, S(═O)₂R^{k}, NR^{m}Rⁿ, N(OR^{k})R^{m}, S(═O)₂NR^{m}Rⁿ, O—C(═O)R^{k}, C(═O)R^{k}, C(═O)OR^{k}, C(═O)NR^{m}Rⁿ, C(═NOR^{k})R^{m}, NR^{k}C(=G)R^{m}, N(OR^{k})C(=G)R^{m}, N[C(=G)R^{k}][C(=G)Rⁿ], NR^{k}C(=G)OR^{m}, N(OR^{k})C(=G)OR^{m}, C(=G)NR^{k}—NR^{m}Rⁿ, NR^{k}SO₂R^{m}, PR^{m}Rⁿ, SiR^{k}_{z}R^{m}_{3-z} with z being 0, 1, 2 or 3,
phenyl and a 3-, 4-, 5-, 6- or 7-membered heterocycle containing 1, 2, 3 or 4 heteroatoms selected from the group consisting of oxygen, nitrogen and sulfur, wherein said phenyl or said heterocycle may be unsubstituted or may carry 1, 2, 3, 4 or 5 radicals selected from halogen and C₁-C₄-alkyl;
G is oxygen or sulfur;
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, phenyl, benzyl or a 3-, 4-, 5-, 6- or 7-membered heterocycle containing 1, 2, 3 or 4 heteroatoms as ring members, selected from the group consisting of oxygen, nitrogen and sulfur, wherein C₃-C₈-cycloalkyl, C₃-C₈-cycloalkenyl, phenyl, benzyl and said heterocycle may be unsubstituted or may carry 1, 2, 3, 4 or 5 radicals selected from halogen and C₁-C₄-alkyl,
or R^{m} and Rⁿ together with the nitrogen, to which they are bound may form an N-bound 5-, 6- or 7-membered heterocycle, which in addition to the nitrogen atom may contain a further heteroatom selected from the group consisting of oxygen, nitrogen and sulphur,
R^{b} is halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, benzyl or a radical R^{a};
or, if X is not a chemical bond, R⁶ may also be a radical of the formulae OR^{c}, SR^{c}, S(═O)R^{c}, S(═O)₂R^{c}, NR^{d}R^{e}, N(OR^{c})R^{d}, S(═O)₂NR^{d}R^{e}, C(═O)R^{c}, C(═O)OR^{c}, O—C(═O)R^{c}, C(═O)NR^{d}R^{e}, C(═O)NR^{c}—NR^{d}R^{e}, C(═NOR^{c})R^{d}, C(═S)R^{c}, C(═S)OR^{c}, C(═S)NR^{d}R^{e}, NR^{d}C(=G)R^{c}, N(OR^{c})C(=G)R^{d}, N[C(=G)R^{c}][C(=G)R^{d}], NR^{d}C(=G)OR^{c}, N(OR^{c})C(=G)OR^{d}, NR^{k}SO₂R^{m} or SiR^{k}_{z}R^{m}_{3-z} with z being 0, 1, 2 or 3,
R^{c}, R^{d}, R^{e} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, phenyl, benzyl or a 3-, 4-, 5-, 6- or 7-membered heterocycle containing 1, 2, 3 or 4 heteroatoms a ring members, selected from the group consisting of oxygen, nitrogen and sulfur, wherein C₃-C₆-cycloalkyl, C₃-C₆-cycloalkenyl, phenyl, benzyl and said heterocycle may be unsubstituted or may carry 1, 2, 3, 4 or 5 radicals selected from halogen and C₁-C₄-alkyl,
or R^{d} and R^{e} together with the nitrogen, to which they are bound may form an N-bound 5-, 6- or 7-membered heterocycle, which in addition to the nitrogen atom may contain a further heteroatom selected from the group consisting of oxygen, nitrogen and sulphur;
R⁷ is hydrogen, C₁-C₆-alkyl, C₂-C₆-alkenyl, C₂-C₆-alkynyl, wherein the last three mentioned radicals may be unsubstituted or may carry 1, 2 or 3 identical or different radicals R^{f}, wherein R^{f} has the meanings given for R^{a},
C₁-C₆-haloalkyl, C₂-C₆-haloalkenyl, C₂-C₆-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from the group consisting of oxygen, nitrogen and sulphur, it being possible for said heterocycle to contain one carbonyl group as ring member, wherein C₃-C₆-cycloalkyl, C₃-C₆-cycloalkenyl, phenyl and said heterocycle may be unsubstituted or may carry 1, 2, 3, 4 or 5 substituents R^{g}, wherein R^{g} has the meanings given for R^{b},
wherein C₃-C₈-cycloalkyl, C₃-C₈-cycloalkenyl, phenyl and said heterocycle may also be fused to a ring selected from the group consisting of phenyl and a 5- to 6-membered saturated, partially unsaturated or aromatic heterocycle which may contain 1, 2 or 3 heteroatoms selected from the group consisting of oxygen, nitrogen, sulfur,
and a radical of the formulae OR^{c}, SR^{c}, S(═O)R^{c}, S(═O)₂R^{c}, NR^{d}R^{e}, N(OR^{c})R^{d}, S(═O)₂NR^{d}R^{e}, C(═O)R^{c}, C(═O)OR^{c}, C(═O)NR^{d}R^{e}, C(═O)NR^{c}—NR^{d}R^{e}, C(═NOR^{c})R^{d}, C(═S)R^{c}, C(═S)OR^{c}, C(═S)NR^{d}R^{e}, NR^{d}C(=G)R^{c}, N(OR^{c})C(=G)R^{d}, N[C(=G)R^{c}][C(=G)R^{d}], NR^{d}C(=G)OR^{c}, N(OR^{c})C(=G)OR^{d}, NR^{k}SO₂R^{m}, SiR^{k}_{z}R^{m}_{3-z}, wherein G, n, R^{c}, R^{d}, R^{e}, R^{k} and R^{m} are as defined above.

51. A method of claim 31, wherein said compounds of formula I or the salt thereof, respectively, is applied at a rate of 5 g/ha to 2000 g/ha.

52. A method of protection of seed comprising contacting the seeds with at least one compound of formulae (I) or an agriculturally acceptable salt thereof, 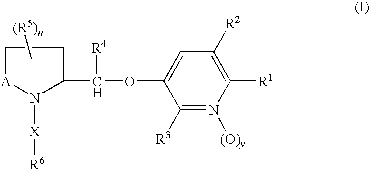
wherein
A is a chemical bond, CH₂ or a group CHR^{5a};
X is a chemical bond or C₁-C₃-alkylene, which may carry 1 or 2 identical or different radicals R⁷;
y is 0 or 1;
n is 0, 1 or 2;
R¹ and R² are each independently selected from the group consisting of hydrogen, halogen, cyano, nitro, C₁-C₆-alkyl, C₁-C₆-alkyl, which may be substituted by one OH, phenyl or CN radical, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, C₁-C₆-alkyl-C₃-C₈-cycloalkyl, ORⁱ, SRⁱ, S(═O)Rⁱ, S(═O)₂Rⁱ, NRⁱR^{j}, NR^{j}C(═O)Rⁱ, C(═O)Rⁱ, O—C(O)Rⁱ, C(═O)ORⁱ, SiRⁱ_{z}R^{j}_{3-z} with z being 0, 1, 2 or 3,
phenyl and a 3-, 4-, 5-, 6- or 7-membered heterocycle containing 1, 2, 3 or 4 heteroatoms selected from the group consisting of oxygen, nitrogen and sulfur, wherein phenyl and said heterocycle may be unsubstituted or may carry 1, 2, or 3 substituents selected from the group consisting of halogen, amino, cyano, Rⁱ, ORⁱ, SRⁱ, NRⁱR^{j}, NR^{j}C(═O)Rⁱ, C(═O)Rⁱ, O—C(O)Rⁱ, C(═O)ORⁱ, SO₂Rⁱ, and nitro;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, C₁-C₆-haloalkoxy, C₁-C₄-alkoxy-C₁-C₆-alkyl, C₁-C₄-alkylthio-C₁-C₆-alkyl, C₁-C₄-alkylsulfinyl-C₁-C₆-alkyl, C₁-C₄-alkylsulfonyl-C₁-C₆-alkyl, C₁-C₄-haloalkoxy-C₁-C₆-alkyl, C₁-C₄-haloalkylthio-C₁-C₆-alkyl, (C₁-C₄-alkoxy)carbonyl-C₁-C₆-alkyl, (C₁-C₄-alkyl)amino-C₁-C₆-alkyl, di(C₁-C₄-alkyl)amino-C₁-C₆-alkyl, C₃-C₆-cycloalkyl-C₁-C₆-alkyl, phenyl-C₁-C₆-alkyl, or C₁-C₆-alkyl which is substituted with 1 to 3 cyano groups;
R² may also be a radical of the formula Z—R^{y}, wherein Z is selected from the group consisting of —C≡C—(C₀-C₆-alkylene)-, —(CH═CH)ₚ—(C₀-C₆-alkylene)- with p being 1 or 2, —CH═CH—C(O)—(C₀-C₆-alkylene)-, —C(O)—(C₀-C₆-alkylene)- and —CH₂—Z′—C(O)—(C₀-C₆-alkylene)- with Z' being CH₂ or NH and R^{y} is selected from the group consisting of hydrogen, C₁-C₈-alkyl, C₁-C₆-haloalkyl, C₁-C₆-hydroxyalkyl, C₁-C₆-alkoxy, amino, C₁-C₆-alkylamino, C₁-C₆-dialkylamino, 5 to 7 membered, azaheterocyclyl, attached to Z via its nitrogen atom, phenyl, naphthyl, biphenyl, furyl, thienyl, pyridinyl, pyridazinyl, pyrimidinyl, pyrrolyl, pyrazolyl, imidazolyl, indolyl, thiazolyl, oxazolyl, isoxazolyl, thiadiazolyl, oxadiazolyl, quinolinyl, and isoquinolyl, wherein alkyl and the 19 last mentioned radicals may carry one or two substituents selected from the group consisting of C₁-C₆-alkyl, C₁-C₆-haloalkyl, C₁-C₆-alkoxy-C₁-C₆-alkyl, C₁-C₆-alkoxy, C₁-C₆-alkoxy-C₁-C₆-alkoxy, halogen, cyano, hydroxy, amino, C₁-C₆-alkylamino, carboxyl and C₁-C₆-alkoxycarbonyl;
R³ is hydrogen, halogen or C₁-C₆-alkyl;
R⁴ is hydrogen or C₁-C₆-alkyl;
R⁵ is selected from the group consisting of halogen, OH, cyano, C₁-C₆-alkyl and C₁-C₆-haloalkyl and for n being 2 R⁵ may be identical or different;
R^{5a} is selected from the group consisting of halogen, cyano, C₁-C₆-alkyl and C₁-C₆-haloalkyl;
R⁶ is C₁-C₁₀-alkyl, C₂-C₁₀-alkenyl, C₂-C₁₀-alkynyl, wherein the last three mentioned radicals may be unsubstituted or may carry 1, 2 or 3 identical or different radicals R^{a},
C₁-C₁₀-haloalkyl, C₂-C₁₀-haloalkenyl, C₂-C₁₀-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from the group consisting of oxygen, nitrogen and sulfur, wherein C₃-C₈-cycloalkyl, C₃-C₈-cycloalkenyl, phenyl and said heterocycle may be unsubstituted or may carry 1, 2, 3, 4 or 5 substituents R^{b},
wherein C₃-C₈-cycloalkyl, C₃-C₈-cycloalkenyl, phenyl and said heterocycle may also be fused to 1 or 2 rings selected from the group consisting of phenyl and a 5- to 6-membered heterocycle which may contain 1, 2 or 3 heteroatoms selected from oxygen, nitrogen and sulfur,
R^{a} is selected from the group consisting of cyano, nitro, C₃-C₈-cycloalkyl, C₃-C₈-halocycloalkyl, C₃-C₈-cycloalkenyl, C₃-C₈-halocycloalkenyl, OR^{k}, SR^{k}, S(═O)R^{k}, S(═O)₂R^{k}, NR^{m}Rⁿ, N(OR^{k})R^{m}, S(═O)₂NR^{m}Rⁿ, O—C(═O)R^{k}, C(═O)R^{k}, C(═O)OR^{k}, C(═O)NR^{m}Rⁿ, C(═NOR^{k})R^{m}, NR^{k}C(=G)R^{m}, N(OR^{k})C(=G)R^{m}, N[C(=G)R^{k}][C(=G)R^{m}], NR^{k}C(=G)OR^{m}, N(OR^{k})C(=G)OR^{m}, C(=G)NR^{k}—NR^{m}Rⁿ, NR^{k}SO₂R^{m}, PR^{m}Rⁿ, SiR^{k}_{z}R^{m}_{3-z} with z being 0, 1, 2 or 3,
phenyl and a 3-, 4-, 5-, 6- or 7-membered heterocycle containing 1, 2, 3 or 4 heteroatoms selected from the group consisting of oxygen, nitrogen and sulfur, wherein said phenyl or said heterocycle may be unsubstituted or may carry 1, 2, 3, 4 or 5 radicals selected from halogen and C₁-C₄-alkyl;
G is oxygen or sulfur;
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkenyl, C₃-C₈-halocycloalkenyl, phenyl, benzyl or a 3-, 4-, 5-, 6- or 7-membered heterocycle containing 1, 2, 3 or 4 heteroatoms as ring members, selected from the group consisting of oxygen, nitrogen and sulfur, wherein C₃-C₈-cycloalkyl, C₃-C₈-cycloalkenyl, phenyl, benzyl and said heterocycle may be unsubstituted or may carry 1, 2, 3, 4 or 5 radicals selected from halogen and C₁-C₄-alkyl,
or R^{m} and R^{a} together with the nitrogen, to which they are bound may form an N-bound 5-, 6- or 7-membered heterocycle, which in addition to the nitrogen atom may contain a further heteroatom selected from the group consisting of oxygen, nitrogen and sulphur,
R^{b} is halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, benzyl or a radical R^{a};
or, if X is not a chemical bond, R⁶ may also be a radical of the formulae OR^{c}, SR^{c}, S(═O)R^{c}, S(═O)₂R^{c}, NR^{d}R^{e}, N(OR^{c})R^{d}, S(═O)₂NR^{d}R^{e}, C(═O)R^{c}, C(═O)OR^{c}, O—C(═O)R^{c}, C(═O)NR^{d}R^{e}, C(═O)NR^{c}—NR^{d}R^{e}, C(═NOR^{c})R^{d}, C(═S)R^{c}, C(═S)OR^{c}, C(═S)NR^{d}R^{e}, NR^{d}C(=G)R^{c}, N(OR^{c})C(=G)R^{d}, N[C(=G)R^{c}][C(=G)R^{d}], NR^{d}C(=G)OR^{c}, N(OR^{c})C(=G)OR^{d}, NR^{k}SO₂R^{m} or SiR^{k}_{z}R^{m}_{3-z} with z being 0, 1, 2 or 3,
R^{c}, R^{d}, R^{e} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, phenyl, benzyl or a 3-, 4-, 5-, 6- or 7-membered heterocycle containing 1, 2, 3 or 4 heteroatoms a ring members, selected from the group consisting of oxygen, nitrogen and sulfur, wherein C₃-C₆-cycloalkyl, C₃-C₆-cycloalkenyl, phenyl, benzyl and said heterocycle may be unsubstituted or may carry 1, 2, 3, 4 or 5 radicals selected from halogen and C₁-C₄-alkyl,
or R^{d} and R^{e} together with the nitrogen, to which they are bound may form an N-bound 5-, 6- or 7-membered heterocycle, which in addition to the nitrogen atom may contain a further heteroatom selected from the group consisting of oxygen, nitrogen and sulphur;
R⁷ is hydrogen, C₁-C₆-alkyl, C₂-C₆-alkenyl, C₂-C₆-alkynyl, wherein the last three mentioned radicals may be unsubstituted or may carry 1, 2 or 3 identical or different radicals R^{f}, wherein R^{f} has the meanings given for R^{a},
C₁-C₆-haloalkyl, C₂-C₆-haloalkenyl, C₂-C₆-haloalkynyl,
C₃-C₈-cycloalkyl, C₃-C₈-cycloalkenyl, phenyl or a 3-, 4-, 5-, 6- or 7-membered heterocycle containing 1, 2, 3 or 4 heteroatoms selected from the group consisting of oxygen, nitrogen and sulphur, it being possible for said heterocycle to contain one carbonyl group as ring member, wherein C₃-C₆-cycloalkyl, C₃-C₆-cycloalkenyl, phenyl and said heterocycle may be unsubstituted or may carry 1, 2, 3, 4 or 5 substituents R^{g}, wherein R^{g} has the meanings given for R^{b},
wherein C₃-C₈-cycloalkyl, C₃-C₈-cycloalkenyl, phenyl and said heterocycle may also be fused to a ring selected from the group consisting of phenyl and a 5- to 6-membered saturated, partially unsaturated or aromatic heterocycle which may contain 1, 2 or 3 heteroatoms selected from the group consisting of oxygen, nitrogen, sulfur,
and a radical of the formulae OR^{c}, SR^{c}, S(═O)R^{c}, S(═O)₂R^{c}, NR^{d}R^{e}, N(OR^{c})R^{d}, S(═O)₂NR^{d}R^{e}, C(═O)R^{c}, C(═O)OR^{c}, C(═O)NR^{d}R^{e}, C(═O)NR^{c}—NR^{d}R^{e}, C(═NOR^{c})R^{d}, C(═S)R^{c}, C(═S)OR^{c}, C(═S)NR^{d}R^{e}, NR^{d}C(=G)R^{c}, N(OR^{c})C(=G)R^{d}, N[C(=G)R^{c}][C(=G)R^{d}], NR^{d}C(=G)OR^{c}, N(OR^{c})C(=G)OR^{d}, NR^{k}SO₂R^{m}, SiR^{k}_{z}R^{m}_{3-z}, wherein G, n, R^{c}, R^{d}, R^{e}, R^{k} and R^{m} are as defined above.

53. The method of claim 52, wherein said at least one compound of formula (I) and/or a N-oxide or an agriculturally acceptable salt thereof is applied in an amount of from 0.1 g to 10 kg per 100 kg of seeds.

54. The method of claim 31, wherein said pests are insects.

55. Seed, comprising at least one compound of formula (I) and/or a N-oxide or an agriculturally acceptable salt thereof as defined in claim 52.

56. A pesticidal composition, which comprises at least one compound of the formula I of claim 31 or a salt thereof and an agriculturally acceptable carrier.

57. The composition of claim 56, which is formulated into dusting powders or granules, dispersible powders, granules or grains, aqueous dispersions, suspensions, pastes, or emulsions.

58. Compounds of formula I wherein A, X, R¹, R², R³, R⁴, R⁵, R⁶, R⁷, n and y are as defined in claim 31, provided that X—R⁶ is not CH₃,
further provided that the following compounds and their salts are excluded
compounds of the formula I, wherein:
R¹ is fluoro; R², R³ and R⁴ are hydrogen; A is a chemical bond;
y and n are zero; and the moiety X—R⁶ is selected from the group consisting of Imidazolidin-2,3-dion-1-ylmethyl (succinimidylmethyl), phthalimide-1-yl, 2-oxo-tetrahydrofuran-4-yl, 2-oxo-2,5-dihydrofuran-4-yl, N-(2-hydroxybenzoyl)aminomethyl, 5,5-dimethyl-3-oxocyclohexenyl, 3-oxocyclohexenyl, 2,2-bis(ethoxycarbonyl)ethenyl, ethyl, n-propyl, n-butyl, 2-propyl, n-pentyl, 2-methylpropan-1-yl, 3,3-dimethylpropin-3-yl and 1,1-dimethylpropan-1-yl;
the compound of the formula I, wherein:
R² is fluoro; R¹, R³ and R⁴ are hydrogen; A is a chemical bond; y and n are zero;
and the moiety X—R⁶ is n-propyl;
compounds of the formula I, wherein:
R¹ is chloro; R², R³ and R⁴ are hydrogen; A is a chemical bond; y and n are zero;
and the moiety X—R⁶ is ethyl, 3,3-dimethylpropin-3-yl or 1,1-dimethylpropan-1-yl;
the compounds of the formula I, wherein:
R¹ is methyl; R², R³ and R⁴ are hydrogen; A is a chemical bond; y and n are zero;
and the moiety X—R⁶ is ethyl;
the compounds of the formula I, wherein:
R¹ is chloro; R², is methyl; R³ and R⁴ are hydrogen; A is a chemical bond; y and n are zero; and the moiety X—R⁶ is n-pentyl, 1,1-dimethylpropan-1-yl or 3,3-dimethylpropin-3-yl;
compounds of the formula I, wherein:
R¹, R², R³ and R⁴ are hydrogen, A is CH₂; y and n are zero; and the moiety X—R⁶ is selected from the group consisting of ethyl, 3-propenyl (allyl), 3-hydroxylpropan-1-yl, 3-fluoropropan-1-yl and 3-(methylsulfonyloxy)propan-1-yl;
the compound of the formula I, wherein:
R² is bromo; R', R³ and Ware hydrogen; A is CH₂; y and n are zero; and the moiety X—R⁶ is 3-propenyl;
the compound of the formula I, wherein:
R¹ is chloro; R², R³ and R⁴ are hydrogen; A is a chemical bond;
y and n are zero; and the moiety X—R⁶ is
3-amino-4-[(2-chloropyridin-5-yl)oxy]butyl.

59. Synergistic pesticidal mixtures, comprising a compound of formula I of claim 31, or a salt thereof, and a pesticide selected from the group consisting of the organo(thio)phosphates, carbamates, pyrethroids, growth regulators, neonicotinoids, nicotinic receptor agonists/antagonists compounds, GABA antagonist compounds, macrocyclic lactone insecticides, METI I, II and III compounds, oxidative phosphorylation inhibitor compounds, moulting disruptor compounds, mixed function oxidase inhibitor compounds, sodium channel blocker compounds, benclothiaz, bifenazate, cartap, flonicamid, pyridalyl, pymetrozine, sulfur, thiocyclam, flubendiamide, cyenopyrafen, flupyrazofos, cyflumetofen, amidoflumet, N—R′-2,2-dihalo-1-R″cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, and anthranilamide compounds of formula Γ³ 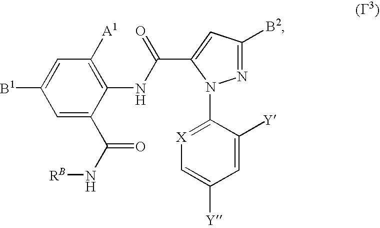
wherein A¹ is CH₃, Cl, Br, I, X is C—H, C—Cl, C—F or N, Y′ is F, Cl, or Br, Y″ is F, Cl, CF₃, B¹ is hydrogen, Cl, Br, I, CN, B² is Cl, Br, CF₃, OCH₂CF₃, OCF₂H, and R^{B} is hydrogen, CH₃ or CH(CH₃)₂.
